(19) United States
(12) Patent Application Publication
(10) Pub. No.: US 2022/0205990 A1
(43) Pub. Date: Jun. 30, 2022

(54) METHOD FOR SMALL-RNA BIOMARKER IDENTIFICATION AND FUNCTIONAL EVALUATION OF CIRCULATING EXTRACELLULAR VESICLES COMPRISING EXOSOMES

(71) Applicant: HACKENSACK MERIDIAN HEALTH CENTER FOR DISCOVERY AND INNOVATION, Nulley, NJ (US)

(72) Inventors: Megan Irvette Mitchell, Clifton, NJ (US); Olivier Loudig, Yorktown Heights, NY (US); Masood Kamali-Moghaddam, Knivsta (SE)

(73) Assignee: HACKENSACK MERIDIAN HEALTH CENTER FOR DISCOVERY AND INNOVATION, Nutley, NJ (US)

(21) Appl. No.: 17/560,909

(22) Filed: Dec. 23, 2021

Related U.S. Application Data
(60) Provisional application No. 63/130,545, filed on Dec. 24, 2020.

(51) Int. Cl. G01N 33/543; C12Q 1/686; G01N 33/544; G01N 33/569; B01D 61/14

(52) U.S. Cl. CPC G01N 33/5432; C12Q 1/686; B01D 61/145; G01N 33/56983; G01N 33/544

(57) ABSTRACT

A method of purifying Extracellular Vesicle Capture by AnTibody of CHoice and Enzymatic Release (EV-CATCHER), designed for high-throughput analysis of low-abundance small-RNA cargos by next-generation sequencing, and use of this method for preparing purified populations of biological particles or cells from a biological sample for in vitro evaluation of a patient's risk of developing and for treating a severe viral infection; for enhancing therapeutic effectiveness of convalescent plasma therapy in a patient at risk for a severe coronavirus infection, and for treating a patient with a severe coronavirus infection, are described.

### CROSS-REFERENCE TO RELATED APPLICATIONS

This application claims the benefit of priority to U.S. Provisional Application No. 63/130,545, filed Dec. 24, 2020, entitled “Methods for Small-RNA Biomarker Identification and Functional evaluation of Circulating Extracellular Vesicles Comprising Exosomes”. The entire contents of the aforementioned application is incorporated by reference herein in its entirety.

### SEQUENCE LISTING

The instant application contains a Sequence Listing which has been submitted electronically in ASCII format and is hereby incorporated by reference in its entirety. Said ASCII copy, created on Dec. 22, 2021 is named 129642-00402_SL.txt and is 21,261 bytes in size.

### FIELD OF THE DISCLOSURE

The present disclosure relates to methods of isolation and purification of circulating extracellular vesicles.

### BACKGROUND OF THE INVENTION

Nucleic acids released by cells during infection, inflammation, cancer, and other physiological and pathological processes can be found circulating in human blood and represent potentially powerful biomarkers of disease [1,2]. Technologies detecting circulating cell-free genomic tumor DNA mutations are showing great promise to evaluate treatment response and presence of residual disease in cancer for example [3-6]. While large single-stranded RNA transcripts are degraded in circulation, small-RNA molecules, and in particular microRNAs (miRNA; ˜22 nt long), remain intact and can be detected and measured to reflect pathological processes. miRNAs are master transcriptional regulators that modulate the activity of specific mRNA targets and play important roles in a wide range of normal and pathological processes [7-12]. Despite significant progress in detection of circulating miRNAs, the discovery of disease-related miRNA biomarkers has remained a challenge due to the low representation of these molecules in the large pool of circulating miRNAs from diverse cellular and tissue sources, therefore requiring higher detection sensitivity and specificity. To address this issue, studies are now focused on the analysis of extracellular vesicles-encapsulated miRNA cargos rather than total RNA purified from blood, serum, or plasma [13-15]. Extracellular vesicles, which are actively released by most cell types into the microenvironment and the circulation, are known to participate in intercellular communication, via target cell uptake, and the miRNAs that they transport have been implicated in the re-programming of recipient cells [16-20].

Extracellular vesicles (EVs) with bilipid layers are differentiated by both their unique size range (30-150 nm in diameter) and biogenesis [21]. They are actively secreted and specifically enriched in the membrane-bound tetraspanins CD9, CD63, CD81, CD37, and CD82, which can be targeted for antibody purification [21,22]. During their secretion, extracellular vesicles also acquire surface molecules (e.g., proteins, lipids) from their cell of origin, which can also be targeted for a more selective antibody-based purification [23-25]. Although major advances have been made over the last few years in the development of microfluidic-based systems for extracellular vesicle purification, very few have reached the commercial market and thus laboratory-based techniques for the purification and evaluation of extracellular vesicles from biofluids remains the gold standard. To date, four principal circulating extracellular vesicle purification approaches have been developed, with a majority of them providing an averaged biomarker evaluation via bulk extracellular vesicle selection [26-28]. Three classical approaches to purifying extracellular vesicles rely on ultracentrifugation, precipitation, and size-exclusion [26-29]. Although these methods result in pure extracellular vesicle preparations (with caveats for precipitation-based methods), all of them produce a mix of extracellular vesicles sub-populations released into circulation by various tissues and cell types, confounding subsequent analyses. The fourth method employs magnetic beads coated with monoclonal antibodies for immuno-purification of extracellular vesicles by selection of their surface biomarkers [25, 30, 31]. Although streptavidin magnetic-beads may allow for a customizable biotinylated antibody-based selection, this customization remains challenging to optimize and is highly dependent on the quality (surface polymer) and design (streptavidin coating) of the magnetic beads. Generally, most of the existing commercially available antibody-based purification assays are static systems, which rely uniquely on the recognition of known extracellular vesicle surface-markers (i.e. CD63, CD81 and CD9) and as such do not allow for the customizable selection and evaluation of different sub-populations of cell-specific extracellular vesicles. Although these pre-established assays allow for robust extracellular vesicle selection, a customizable antibody-selection assay whereby cell-specific surface markers may be selected from a biofluid holds greater promise for identification of circulating disease biomarkers [32]. Finally, rarely are antibody-based methods built to allow for the intact release of selected extracellular vesicles for downstream in vitro analyses. A recent immunoaffinity-based extracellular vesicle isolation assay (exo-PLA) addressed many of these issues by using an enzymatically degradable DNA linker between the extracellular vesicle-binding antibody and the magnetic bead, permitting release of intact extracellular vesicles [31]. However, our initial evaluation indicated that magnetic beads, used with this approach, could non-specifically bind small-RNAs to a degree significantly limiting detection of low-abundance miRNA cargos.

In addition to representing a valuable source of biomarkers, extracellular vesicles are also key players in multiple biological processes, particularly in the immune system [33-38]. Recent studies have suggested that extracellular vesicles participate in antigen presentation to the immune system [37,38] and may mediate immune defense during viral infection [33,34]. The mechanistic role that extracellular vesicles play in immunity against well studied viruses (HIV, EBV, Ebola) has previously been evaluated [39-41]. However, few studies have evaluated their potential implications for SARS-CoV-2 infections. Our health care network (Hackensack Meridian Health) is located within the U.S. epicenter of the SARS-CoV-2 pandemic, and nearly half of all New Jersey Covid-19 patients were hospitalized in our hospitals. This allowed us to gain access to a collection of serum samples from Covid-19 patients and use it for a proof-of principle study applying our new extracellular vesicle purification assay to clinical specimens.

### SUMMARY OF THE INVENTION

The described invention provides a method of purifying exosomes and/or extracellular vesicles, the Extracellular Vesicle Capture by AniTbody of CHoice and Enzymatic Release (EV-CATCHER) assay. We describe the step-by-step design and evaluation of this assay for the customizable selection and release of intact circulating extracellular vesicles. Combining the EV-CATCHER assay with our highly sensitive small-RNA cDNA library preparation protocol [14,42], for high throughput small-RNA sequencing, we demonstrate its applicability by purifying circulating extracellular vesicles from sera of Covid-19 patients and identifying differentially expressed miRNAs associated with severity of the disease between hospitalized patients. Additionally, upon evaluating the integrity of extracellular vesicles purified and released by EV-CATCHER, we confirmed neutralizing activity of convalescent extracellular vesicles from sera of recovered Covid-19 individuals, unexpectedly identified using ultracentrifuge purified extracellular vesicles.

According to one aspect, the present disclosure provides a method of preparing a purified population of biological particles from a biological sample from a subject and for evaluating a cargo of the purified population of biological particles comprising: a) preparing a purified population of biological particles by: (1) obtaining a biological sample comprising biological particles; (2) contacting the biological sample comprising biological particles from the subject with a binding agent directed to one or more biological particle surface antigens; wherein the binding agent is linked to a nucleic acid, and wherein the nucleic acid is immobilized on a solid support; b) isolating the biological particle bound by the binding agent from the biological sample; c) releasing the biological particle bound to the binding agent; d) eluting the bound biological particle from the binding agent to form a population of free purified biological particles; and e) evaluating cargo and surface molecules comprising protein, DNA, RNA or lipids, of the purified population of biological particles.

According to some embodiments, step (e) evaluating cargo and surface molecules further comprises one or more of: (i) identifying proteins specific to a surface of the biological particles by mass spectrometry; or (ii) identifying protein cargos by mass spectrometry; or (iii) identifying DNA molecules by sequencing or quantitative PCR; or (iv) extracting RNA from the purified population of biological particles, and identifying and quantifying expression of small non-coding RNAs comprising microRNAs (miRNAs) encapsulated by the purified population of biological particles.

According to some embodiments, the method comprises an initial ultrafiltration or ultracentrifugation step to provide a starting pooled heterogeneous population of biological particles. According to some embodiments, the biological sample comprises a body fluid. According to some embodiments, the body fluid comprises whole blood, serum, plasma, cerebrospinal fluid (CSF), lymph, urine, feces, sweat, tears, nipple aspirates, or seminal fluid or a secreted biological fluid. According to some embodiments, the body fluid is a circulating/secreted body fluid. According to some embodiments, the circulating body fluid is whole blood, serum, plasma, cerebrospinal fluid (CSF) or lymph.

According to some embodiments, the binding agent that binds to one or more biological particle surface antigens is an antibody, an antibody binding fragment, or an aptamer. According to some embodiments, the aptamer is a nucleic acid or a polypeptide. According to some embodiments, the biological particle surface antigen comprises one or more of CD9, CD63, CD81, CD37, CD82, Alix, ACE-2, Tim4, PLAP, Adiponectin, FABP4, Caveolin-1, Cytokeratins, EPCAM, E-Cadherin, P63, heterologous cell surface polypeptides and/or other cell surface markers inherited by the biological particles. According to some embodiments, the nucleic acid comprises DNA, RNA, or a combination thereof. According to some embodiments, the nucleic acid comprises DNA. According to some embodiments, the DNA comprises one or more ribonucleic acid nucleotide. According to some embodiments, the one or more ribonucleic acid nucleotide is uracil. According to some embodiments, the DNA comprises a restriction enzyme recognition site. According to some embodiments, the nucleic acid comprises non-natural nucleotides. According to some embodiments, the nucleic acid further comprises a binding moiety on a first end of the nucleic acid and a binding moiety on a second end of the nucleic acid, and wherein the binding moiety on the first end of the nucleic acid and the binding moiety on the second end of the nucleic acid are different. According to some embodiments, the binding moiety on the first end of the nucleic acid is an avidin, streptavidin or carboxyl binding moiety. According to some embodiments, the binding moiety is biotin. According to some embodiments, the binding moiety on the second end of the nucleic acid is an amine moiety. According to some embodiments, the amine moiety is azide. According to some embodiments, the binding agent to one or more biological particle surface antigens comprises a dibenzocyclooctyne (DBCO) molecule, 2-IT (2-iminothiolane), MBS (3-maleimidobenzoic acid N-hydroxysuccinimide ester), SPDP (N-succinimidyl 3-(2-pyridyldithio) propionate), SATA (N-succinimidyl S-acetylthioacetate), SMCC (succinimidyl 4-(N-maleimidomethyl) cyclohexane-1-carboxylate), Sulfo-SMCC, or derivatives thereof. According to some embodiments, the solid support is a well plate, polymer, or a surface. According to some embodiments, releasing the isolated biological particle comprises: (i) enzymatically cleaving the nucleic acid; or (ii) displacing a first strand of the nucleic acids connected to the antibody from the second strand of the nucleic acids connected to the support by strand displacement with a complementary nucleic acid to the first or second strand of the nucleic acid and an enzyme having strand displacement activity to release the antibody from the support; or (iii) separating the annealed DNA strands to allow release of the antibody from the platform without damaging the DNA strand attached to the antibody by a polymerase chain reaction using an oligonucleotide complementary to the region of the DNA attached to the antibody. According to some embodiments, the enzymatic cleaving is with uracil glycosylase. According to some embodiments, the enzymatic cleaving is with a restriction enzyme. According to some embodiments, the enzyme having strand displacement activity is DNA polymerase, topoisomerase, or helicase. According to some embodiments, the method comprises identifying the one or more small non-coding RNAs comprising miRNAs encapsulated in the one or more biological particle by next generation sequencing. According to some embodiments, the isolated biological particles are derived from a healthy subject or subject suffering from a disease. According to some embodiments, the disease comprises a viral infection, cancer, abnormal placentation, exercise induced muscle damage, heart failure, Alzheimers disease, liver cirrhosis, viral and bacterial infection, kidney disease, bone remodeling after injury, wound healing or COPD and asthma. According to some embodiments, the viral infection is a severe coronavirus infection. According to some embodiments, the severe coronavirus infection is due to SARS-CoV-1, MERS, or SARS-CoV-2. According to some embodiments, the severe coronavirus infection is due to SARS-CoV-2. According to some embodiments, the isolated and quantified miRNAs derived from a subject suffering from a severe coronavirus infection due to SARS-CoV-2 include one or more of hsa-miR-146a, hsa-miR-126-3p, hsa-miR-15a, hsa-miR-424, hsa-miR-151-3p, hsa-miR-126-5p, hsa-miR-627-5p, hsa-miR-145, hsa-miR-205, hsa-miR-200c, hsa-miR-550-5p, and hsa-miR-629. According to some embodiments, miRNA markers of severe SARSCoV-2 disease include hsa-miR-146a, hsa-miR126-3p or both.

According to another aspect, the present disclosure provides a method for in vitro evaluation of a subject's risk of developing and for treating a severe coronavirus infection, comprising: a. preparing a purified population of biological particles by: (1) obtaining a biological sample comprising biological particles from the subject; (2) contacting the biological sample comprising biological particles from the subject with a binding agent to one or more biological particles surface antigens, wherein the binding agent is linked to a nucleic acid and wherein the nucleic acid is immobilized on a solid support; b. isolating the biological particles bound by the binding agent from the biological sample; c. releasing the biological particles bound by the binding agent; d. eluting the bound biological particles from the binding agent to form a population of free purified biological particles; e. determining a cargo profile for the purified biological particles by evaluating cargo of the purified population of biological particles by: (i) extracting RNA from the purified population of biological particles; (ii) identifying and quantifying expression of small non-coding RNAs comprising one or more microRNAs (miRNAs) encapsulated by the purified population of exosomes; (iii) comparing the cargo profile for the purified biological particle to a cargo profile from a control subject (1) not infected with the coronavirus; (2) infected with the coronavirus who developed mild disease; and (3) infected with the coronavirus who developed severe disease; (f) determining risk of the patient for the severe viral infection, wherein the small noncoding RNA or protein cargo profile for the purified biological particle is about 1.5-fold lower or 1.5-fold higher than the cargo profile from the control subject infected with the coronavirus who developed severe disease; and (g) implementing a therapy appropriate for patients at risk for the severe viral infection.

According to some embodiments, the method comprises an initial ultrafiltration or ultracentrifugation step to provide a pooled heterogeneous population of biological particles. According to some embodiments, the biological sample comprises a body fluid. According to some embodiments, the body fluid comprises whole blood, serum, plasma, cerebrospinal fluid (CSF), lymph, urine, feces, sweat, tears, nipple aspirates, seminal fluid or a secreted biological fluid. According to some embodiments, the body fluid is a circulating/secreted body fluid. According to some embodiments, the circulating body fluid is whole blood, serum, plasma, CSF, or lymph. According to some embodiments, the severe coronavirus infection is due to SARS-CoV-1, MERS, or SARS-CoV-2. According to some embodiments, the severe coronavirus infection is due to SARS-CoV-2. According to some embodiments, the miRNAs of a patient infected with SARS-Cov-2 include one or more of hsa-miR-146a, hsa-miR-126-3p, hsa-miR-15a, hsa-miR-424, hsa-miR-151-3p, hsa-miR-126-5p, hsa-miR-627-5p, hsa-miR-145, hsa-miR-205, hsa-miR-200c, hsa-miR-550-5p, and hsa-miR-629 when compared to a control. According to some embodiments, miRNA markers of severe disease caused by SARS-CoV-2 include hsa-miR-146a, hsa-miR126-3p or both when compared to a control. According to some embodiments, the binding agent to one or more biological particles surface antigens is an antibody, an antibody fragment or an aptamer. According to some embodiments, the exosome surface antigen comprises one or more of CD9, CD63, CD81, CD37, CD82, Alix, ACE-2, Tim4, PLAP and/or other cell surface markers inherited by the biological particles. According to some embodiments, the nucleic acid comprises DNA comprising one or more ribonucleic acid nucleotide, and wherein the ribonucleic acid nucleotide is uracil. According to some embodiments, releasing the isolated biological particle comprises: (i) enzymatically cleaving the nucleic acid; or (ii) displacing a first strand of the nucleic acids connected to the antibody from the second strand of the nucleic acids connected to the support by strand displacement with a complementary nucleic acid to the first or second strand of the nucleic acid and an enzyme having strand displacement activity to release the antibody from the support; or (iii) separating the annealed DNA strands to allow release of the antibody from the platform without damaging the DNA strand attached to the antibody by a polymerase chain reaction using an oligonucleotide complementary to the region of the DNA attached to the antibody. According to some embodiments, the enzymatic cleaving is with uracil glycosylase. According to some embodiments, the enzymatic cleaving is with a restriction enzyme. According to some embodiments, the enzyme comprising strand displacement activity is DNA polymerase, topoisomerase, or helicase. According to some embodiments, identifying and quantifying small noncoding RNAs comprising one or more microRNAs (miRNAs) encapsulated by the purified population of biological particles is by next generation sequencing. According to some embodiments, the therapy appropriate for patients at risk for the severe viral infection comprises one or more of a supportive therapy, an antiviral agent, and an immunomodulatory agent. According to some embodiments, (a) the supportive therapy comprises intravenous administration of fluids, supplemental oxygen or inhalation of a vapor by nebulizer; or (b) the antiviral agent inhibits viral entry, decreases viral load or both; or (c) the antiviral agent is selected from acyclovir, gancidovir, foscarnet; ribavirin; amantadine, azidodeoxythymidine (zidovudine), nevirapine, a tetrahydroimidazobenzodiazepinone (TIBO) compound; efavirenz; remdecivir, lopinavir/ritonavir, umifenovir, favipiravir, ivermectin, and delavirdine; or (d) the immunomodulatory agent is a glucocorticoid, or a recombinant interferon; or (e) the immunomodulatory agent is a glucocorticoid, and the glucocorticoid is a corticosteroid. According to some embodiments, the glucocorticoid comprises prednisone, dexamethasone, azathioprine, mycophenolate, mycophenolate mofetil, or combinations thereof.

According to another aspect, the present disclosure provides a method for enhancing therapeutic effectiveness of convalescent plasma therapy for treating a patient at risk for a severe coronavirus or other viral infection comprising: a) preparing a purified population of biological particles from convalescent serum of a convalescent subject by: (1) obtaining a convalescent serum comprising a high IgG titer against the coronavirus from the convalescent subject; (2) contacting the convalescent serum with a binding agent directed to one or more biological particles surface antigens; wherein the binding agent is linked to a nucleic acid, and wherein the nucleic acid is immobilized on a solid support; b) isolating the biological particles bound by the binding agent from the biological sample; c) releasing the biological particles bound to the binding agent; d) eluting the biological particles from the binding agent to form a population of free purified biological particles; e) measuring a neutralization titer of the purified biological particles population for the coronavirus in vitro; and f) administering to the subject the convalescent serum comprising a high titer of neutralizing biological particles and a high titer of neutralizing IgG.

According to some embodiments, the method comprises an initial ultrafiltration or ultracentrifugation step to provide pooled a heterogeneous population of biological particles. According to some embodiments, the biological sample comprises a body fluid. According to some embodiments, the body fluid comprises whole blood, serum, plasma, cerebrospinal fluid (CSF), lymph, urine, feces, sweat, tears, nipple aspirates, seminal fluid or a secreted biological fluid. According to some embodiments, the body fluid is circulating body fluid. According to some embodiments, the circulating body fluid is whole blood, serum, plasma, cerebrospinal fluid (CSF) or lymph.

According to some embodiments, the binding agent to one or more biological particle surface antigens is an antibody, an antibody fragment or an aptamer. According to some embodiments, the biological particle surface antigen comprises one or more of CD9, CD63, CD81, CD37, CD82, Alix, ACE-2, Tim4, PLAP, Adiponectin, FABP4, Caveolin-1, Cytokeratins, EPCAM, E-Cadherin, P63, heterologous cell surface polypeptides, and/or other cell surface markers inherited by the biological particles. According to some embodiments, the nucleic acid comprises DNA comprising a ribonucleic acid nucleotide, wherein the ribonucleic acid nucleotide is uracil, or the DNA comprises a restriction enzyme recognition site. According to some embodiments, releasing the isolated biological particle comprises: (i) enzymatically cleaving the nucleic acid; or (ii) displacing a first strand of the nucleic acids connected to the antibody from the second strand of the nucleic acids connected to the support by strand displacement with a complementary nucleic acid to the first or second strand of the nucleic acid and an enzyme having strand displacement activity to release the antibody from the support; or (iii) separating the annealed DNA strands to allow release of the antibody from the platform without damaging the DNA strand attached to the antibody by a polymerase chain reaction using an oligonucleotide complementary to the region of the DNA attached to the antibody. According to some embodiments, the enzymatic cleaving is with uracil glycosylase. According to some embodiments, the enzymatic cleaving is with a restriction enzyme. According to some embodiments, the enzyme comprising strand displacement activity is DNA polymerase, topoisomerase, or helicase. According to some embodiments, the severe coronavirus infection is due to SARS-CoV-1, MERS, or SARS-CoV-2. According to some embodiments, the severe coronavirus infection is due to SARS-CoV-2. According to some embodiments, the neutralizing purified biological particle population derived from the convalescent serum with the high IgG titer comprises ACE2-receptors. According to some embodiments, the measuring a neutralization titer of the purified biological particle population for SARS-CoV-2 virus further comprising incubating mammalian cells infected with the coronavirus in vitro with a dilution series of the isolated purified biological particle derived from the convalescent serum with the high IgG titer; and measuring viral particle production compared to a negative control (infected cells without the purified biological particle derived from convalescent serum). According to some embodiments, viral particle production is decreased by at least 10%, at least 20%, at least 30%, at least 40%, at least 50%, at least 60%, at least 70%, at least 80%, at least 85%, at least 90%, at least 95%, or at least 100% when compared to a negative control. According to some embodiments, the convalescent serum comprising neutralizing purified biological particle may enhance effectiveness of convalescent plasma therapy by at least about 2-fold when compared to treatment with convalescent plasma therapy alone; or (b) the neutralizing purified biological particle allows presentation of viral antigens to the immune system.

According to another aspect, the present disclosure provides a method of treating a subject with a severe coronavirus infection, comprising: a) determining a neutralization titer of a population of biological particle purified from a biological sample of the subject by: (1) obtaining the biological sample comprising biological particle; (2) contacting the biological samples comprising biological particles from the subject with a binding agent to one or more biological particle surface antigens; wherein the binding agent is linked to a nucleic acid, and wherein the nucleic acid is immobilized on a solid support; (3) isolating the biological particles bound by the binding agent from the biological sample; (4) releasing the biological particles bound to the binding agent (2); (5) eluting the bound biological particles from the binding agent to form a population of free purified biological particles; (6) determining the neutralization titer of the population of purified biological particle in vitro; b) selecting the subject to receive convalescent plasma therapy comprising neutralizing purified biological particles when the neutralization titer of the population of purified biological particle purified from the subject is insufficient to decrease viral particle production in vitro; c) preparing a purified population of biological particle from convalescent plasma of a convalescent subject by: (i) obtaining a convalescent serum comprising a high anti-coronavirus IgG titer from a convalescent subject; (ii) contacting the convalescent serum with a binding agent directed to one or more biological particle surface antigens; (iii) isolating the biological particle bound by the binding agent from the biological sample; (iv) releasing the biological particle bound to the binding agent; (v) eluting the bound biological particle from the binding agent to form a population of free purified biological particle; d) measuring a neutralization titer of the biological particle population purified from the convalescent serum for SARS-CoV-2 virus, in vitro; and (e) administering to the subject the neutralizing purified biological particle alone or the convalescent serum with high IgG titer comprising the neutralizing purified biological particle.

According to some embodiments, the method comprises an initial ultrafiltration or ultracentrifugation step to provide pooled a heterogeneous population of biological particle. According to some embodiments, the biological sample comprises a body fluid. According to some embodiments, the body fluid comprises whole blood, serum, plasma, cerebrospinal fluid (CSF), lymph, urine, feces, sweat, tears, nipple aspirates, seminal fluid or a secreted biological fluid. According to some embodiments, the body fluid comprises circulating body fluid. According to some embodiments, the circulating body fluid is whole blood, serum, plasma, CSF, or lymph.

According to some embodiments, the binding agent to one or more biological particle surface antigens is an antibody, an antibody fragment or an aptamer. According to some embodiments, the biological particle surface antigen comprises one or more of CD9, CD63, CD81, CD37, CD82, Alix, ACE-2, Tim4, PLAP, Adiponectin, FABP4, Caveolin-1, Cytokeratins, EPCAM, E-Cadherin, P63, heterologous cell surface polypeptides, and/or other cell surface markers inherited by the biological particles. According to some embodiments, releasing the isolated biological particle comprises: (i) enzymatically cleaving the nucleic acid; or (ii) displacing a first strand of the nucleic acids connected to the antibody from the second strand of the nucleic acids connected to the support by strand displacement with a complementary nucleic acid to the first or second strand of the nucleic acid and an enzyme having strand displacement activity to release the antibody from the support; or (iii) separating the annealed DNA strands to allow release of the antibody from the platform without damaging the DNA strand attached to the antibody by a polymerase chain reaction using an oligonucleotide complementary to the region of the DNA attached to the antibody. According to some embodiments, the nucleic acid is DNA comprising a ribonucleic acid nucleotide, the ribonucleic acid nucleotide is uracil or DNA containing a restriction site sequence, or a duplex of RNA and DNA molecules, and the enzymatic cleaving is with uracil glycosylase, or DNases, or RNases. According to some embodiments, the enzymatic cleaving is with a restriction enzyme. According to some embodiments, the enzyme comprising strand displacement activity is DNA polymerase, topoisomerase, or helicase. According to some embodiments, the severe coronavirus infection is due to SARS-CoV-1, MERS, or SARS-CoV-2. According to some embodiments, the severe coronavirus infection is due to SARS-CoV-2.

According to some embodiments, the measuring a neutralization titer (i) of the population of biological particles purified from the subject and (ii) of the purified biological particle population from convalescent serum with high IgG titer for the coronavirus further comprises incubating mammalian cells infected with the coronavirus in vitro with a dilution series of (i) the isolated purified biological particles derived from the subject and (ii) isolated purified biological particles derived from convalescent serum with the high IgG titer; and comparing viral particle production compared to a negative control (infected cells alone). According to some embodiments, a neutralizing population of purified biological particles comprises ACE2-receptors.

According to another aspect, the present disclosure provides a method of preparing a population of purified cells from a biological sample from a subject comprising:
a) preparing a population of purified cells by:
   (1) obtaining a biological sample from the subject comprising cells;
   (2) contacting the biological sample from the subject comprising cells with a binding agent directed to one or more cell surface antigens, wherein the binding agent is linked to a nucleic acid by a linker, and wherein the nucleic acid is immobilized on a solid support;
b) isolating the cell bound by the binding agent from the biological sample;
c) releasing the cell bound to the binding agent;
d) eluting the bound cell from the binding agent to form a population of purified cells.

According to some embodiments, the method comprises an initial ultrafiltration or ultracentrifugation step to provide a starting pooled heterogeneous population of cells.

According to some embodiments, the biological sample is prepared in vivo or in vitro.

According to some embodiments, the biological sample comprises a body fluid. According to some embodiments, the body fluid comprises whole blood, serum, plasma, cerebrospinal fluid (CSF), lymph, urine, feces, sweat, tears, nipple aspirates, or seminal fluid or a secreted biological fluid. According to some embodiments, the body fluid is a circulating/secreted body fluid. According to some embodiments, the circulating body fluid is whole blood, serum, plasma, cerebrospinal fluid (CSF) or lymph.

According to some embodiments, the one or more cell surface antigen(s) comprise(s) CD4, CD8, CD9, CD46, CD63, CD81, CD37, CD82, CD138, CD151, ALix, ACE-2, Tim4, PLAP, Adiponectin, FABP4, Caveolin-1, Cytokeratins, EPCAM, E-Cadherin, P63, heterologous cell surface polypeptides.

According to some embodiments, the heterologous cell surface polypeptides are expressed by chimeric antigen receptor T cells (CAR-T cells).

According to some embodiments, the binding agent that binds to one or more cell surface antigens is an antibody, an antibody binding fragment, or an aptamer. According to some embodiments, the aptamer is a nucleic acid or a polypeptide.

According to some embodiments, the nucleic acid comprises DNA, RNA, or a combination thereof. According to some embodiments, the nucleic acid comprises DNA. According to some embodiments, the DNA comprises one or more ribonucleic acid nucleotide. According to some embodiments, the one or more ribonucleic acid nucleotide is uracil. According to some embodiments, the DNA comprises a restriction enzyme recognition site. According to some embodiments, the nucleic acid is a DNA/RNA duplex which can be degraded by an endonuclease or a RNAse (RNase-H). According to some embodiments, the nucleic acid comprises non-natural nucleotides.

According to some embodiments, the nucleic acid further comprises a binding moiety on a first end of the nucleic acid and a binding moiety on a second end of the nucleic acid, and wherein the binding moiety on the first end of the nucleic acid and the binding moiety on the second end of the nucleic acid are different.

According to some embodiments, the binding moiety on the first end of the nucleic acid is an avidin, streptavidin or carboxyl binding moiety. According to some embodiments, the binding moiety is biotin. According to some embodiments, the binding moiety on the second end of the nucleic acid is an amine moiety. According to some embodiments, the amine moiety is azide.

According to some embodiments, the binding agent to one or more cell surface antigens comprises a dibenzocyclooctyne (DBCO) molecule, 2-IT (2-iminothiolane), MBS (3-maleimidobenzoic acid N-hydroxysuccinimide ester), SPDP (N-succinimidyl 3-(2-pyridyldithio) propionate), SATA (N-succinimidyl S-acetylthioacetate), SMCC (succinimidyl 4-(N-maleimidomethyl) cyclohexane-1-carboxylate), Sulfo-SMCC, or derivatives thereof.

According to some embodiments, the solid support is a well plate, polymer, or a surface.

According to some embodiments, releasing the isolated cell comprises:
(i) enzymatically cleaving the nucleic acid; or
(ii) displacing a first strand of the nucleic acids connected to the antibody from the second strand of the nucleic acids connected to the support by strand displacement with a complementary nucleic acid to the first or second strand of the nucleic acid and an enzyme having strand displacement activity to release the antibody from the support; or
(iii) separating the annealed DNA strands to allow release of the antibody from the platform without damaging the DNA strand attached to the antibody by a polymerase chain reaction using an oligonucleotide complementary to the region of the DNA attached to the antibody.

According to some embodiments, the enzymatic cleaving is with uracil glycosylase. According to some embodiments, the enzymatic cleaving is with a restriction enzyme. According to some embodiments, the enzymatic cleaving is with an endonuclease or a RNAse. According to some embodiments, the enzyme having strand displacement activity is DNA polymerase, topoisomerase, or helicase.

According to some embodiments, the population of cells is derived from non-eukaryotic and eukaryotic species; or healthy normal tissue or diseased tissue; or a murine orthotopic/xenograft/PDX organ, or blood, or a tissue culture

According to some embodiments, the eukaryotic cells are human, mouse, rat, dog, non-human primate, or feline.

According to some embodiments, the population of purified cells are derived from a healthy subject or a subject suffering from a disease. According to some embodiments, the disease comprise a cancer, viral infection, abnormal placentation, inflammation and other pathologies.

According to some embodiments, the cells are endothelial cells, epithelial cells, T-cells, fibroblasts, adipocytes, neuronal cells, tumor cells, blood cells, or cardiac cells.

According to some embodiments, the population of cells is derived from (a) non-eukaryotic and eukaryotic species; or (b) healthy normal tissue or diseased tissue; or (c) a murine orthotopic/xenograft/PDX organ; or (d) blood; or (e) a tissue culture.

According to some embodiments, the viral infection is a severe coronavirus infection. According to some embodiments, the severe coronavirus infection is due to SARS-CoV-1, MERS, or SARS-CoV-2. According to some embodiments, the severe coronavirus infection is due to SARS-CoV-2.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a schematic of a customizable, low background, high-affinity assay for specific immuno-capture and release of circulating extracellular vesicles prior to small-RNA sequencing and identification of miRNA biomarkers: The EV-CATCHER assay (Extracellular Vesicle Capture by AnTibody of CHoice and Enzymatic Release).

FIG. 2A depicts a schematic representation of the EV-CATCHER assay designed for purification of extracellular vesicles from biofluids, which relies on binding of a degradable dsDNA-linker (uracilated 5′-azide oligonucleotide annealed to complementary uracilated 3′-biotin oligonucleotide) to a DBCO-activated antibody and to a streptavidin-coated platform.

FIG. 2B shows acrylamide gel migration of single stranded oligonucleotides and double stranded (ds) hybridized DNA-linker. Single stranded (ss) biotin labeled DNA (lane 2); ssDNA azide (lane 3), dsDNA linker (lane 4), UNG digested dsDNA linker (lane 5) were separated on a 15% non-denaturing PAGE gel.

FIG. 2C shows monoclonal human anti-CD63 antibody (Ab) activation and dsDNA-linker conjugation. Representative image of Coomassie stained non-denaturing PAGE gel (12%) with migration of protein ladder (lane a), 1 μg native anti-CD63 Ab (lane b); dsDNA-linker conjugated to 1 μg DBCO-activated anti-CD63 antibody using increased amounts of dsDNA-linker (lanes c-e), dsDNA-linker-Ab (500 ng-1 μg) digested by UNG (lane f). Experiments were replicated and imaged on a ChemiDoc MP imager. SEQ ID NOs: 1 and 2, respectively, in order of appearance, are disclosed.

FIG. 3A shows an assay evaluating non-specific small-RNA binding to streptavidin-coated magnetic beads and wells. Schematic representation (above) of the experimental procedure for sample collection. A 1×PBS solution containing 1 ng ath-miR-159a RNA oligonucleotide was incubated with wells or beads and four different samples were collected including wash #1, wash #2, wash #3 and a final elution. The two left graphs represent RT-qPCR quantifications obtained using control ath-miR-159a RNA (1 ng; Black bars), washes #1, #2, and #3 (grey bars), and final elution (white bars) for wells (top graph left) and beads (bottom graph left), without RNase-A treatment. The two right graphs represent RT-qPCR quantifications obtained using samples collected in the same fashion, where wells and beads were initially treated with RNase-A.

FIG. 3B shows an assay evaluating MCF-7 extracellular vesicle non-specific binding. The experimental collection procedure is displayed in the schematic (above). The two graphs represent RT-qPCR quantifications of hsa-miR-21 (left) and hsa-miR-200c (right) using total RNA extracted from total extracellular vesicles control (10 μg, Black bars), extracellular vesicles+RNase-A (Dark grey bar), extracellular vesicles+RNase-A after incubation and removal of Dynabeads™ (magnetic beads) (Light grey bar), and extracellular vesicles+RNase-A after incubation with wells and removal (white bars). All qPCR experiments were performed in triplicate for each sample. Data is represented as ΔΔCt with technical replicates (subtraction of ath-miR-159a and 10 μg MCF-7 control).

FIGS. 4A-4C shows Western blot evaluation of extracellular vesicle surface markers using anti-Alix, -CD63, -CD9, and -CD81 antibodies. EV-CATCHER purification of CD63⁺ extracellular vesicles from MCF-7 tissue culture media (FIG. 4A) and human plasma (FIG. 4B) with decreasing total protein inputs before purification. For left and middle Western blots, 3 μg (lane 1), 2.75 μg (lane 2), 2.5 μg (lane 3), 2.25 μg (lane 4), 2 μg (lane 5), 1.75 μg (lane 6), and 1.5 μg (lane 7) total protein was used before EV-CATCHER purification. EV-CATCHER was also used for CD63⁺ extracellular vesicle purification from a serum sample (FIG. 4C), and validated by the four different surface protein antibodies

FIG. 4D shows transmission electron microscopy (TEM) of MCF-7 stock extracellular vesicles (left, MCF-7 Stock) and CD63-purified extracellular vesicles from MCF-7 extracellular vesicle stocks (MCF-7), human plasma (Plasma), and human serum (Serum). Direct magnification of 20,000× and scale bars of 200 nm are represented on the TEM images.

FIG. 4E shows representative particle size distribution characterized by nanoparticle tracking (Spectradyne nCS1 equipped with TS400 microfluidic cartridges) for CD63-purified extracellular vesicles from MCF-7 cell culture media (left graph), human plasma (middle graph), and human serum (right graph). Peak filtering performed for diameter <65 nm and transit times >80 μs. Concentrations representative of particles detected between 65 and 150 nm.

FIG. 5A shows a next-generation sequencing and heat map representation of the top expressed circulating miRNAs in human serum. The heat map includes the top 49 expressed miRNAs and their detectability using decreasing amounts of total RNA extracted from human serum (6 ng, 3 ng, and 1.5 ng, in duplicate).

FIG. 5B shows a heat map representation displaying miRNA expression differences between total RNA extracted from mouse RAWS264.7 extracellular vesicles (first two rows), commercially available whole human plasma (third and fourth rows), and extracellular vesicles obtained by ultracentrifugation from commercially available the same whole human plasma (fifth and sixth rows). Duplicate libraries were obtained using newer (Repeats #1) and older (Repeats #2) 3′ barcoded adapters.

FIG. 5C shows EV-CATCHER specific capture of mouse extracellular vesicles spiked into human plasma. The heat map represents miRNA expression differences between mouse RAWS264.7 extracellular vesicles and human plasma, including the top 58 differentially expressed miRNAs between human plasma and mouse RAWS264.7 extracellular vesicles (column 1 and 2) and the top 50 mouse-specific transcripts (column 5 and 6). Mouse RAWS extracellular vesicles (1 μg) spiked in human plasma (100 μl) were purified from plasma using the EV-CATCHER assay harboring a mouse-specific anti-CD63 antibody (column 2 and 3). Duplicate libraries were obtained using newer (Repeats #1) and older (Repeats #2) 3′ barcoded adapters. Data was analyzed using dedicated Bioconductor packages in the R platform and heat maps were generated using the ‘NMF’ package (a heat map function).

FIG. 6A shows individual box plot analyses of the 10 differentially expressed miRNAs identified from EV-CATCHER purified extracellular vesicles (green square; hsa-miR-146a, hsa-miR-126-3p, hsa-miR-424, hsa-miR-151-3p, hsa-miR-126-5p, hsa-miR-627-5p, hsa-miR-145, hsa-miR-205, and hsa-miR-200c) and 2 differentially expressed miRNAs identified in whole serum (orange square; hsa-miR-550-5p, hsa-miR-629*) of mildly and severely ill Covid-19 hospitalized patients, by next-generation small-RNA sequencing. Extracellular vesicles purified from serum were obtained with EV-CATCHER using a combination of anti-CD63, -CD9, -CD81 antibodies.

FIG. 6B shows a box-plot miRNA read representation of the top 10 differentially expressed miRNAs identified from serum exosomes and/or extracellular vesicles in mildly (n=13) and severely ill Covid-19 hospitalized patients (n=17), by comparison to total miRNA reads.

FIG. 6C shows box plots representations of the miRNA integrative signature (including the top 10 miRNA identified in serum exosomes and/or extracellular vesicles) between the two small-RNA libraries prepared with RNA extracted from exosomes and/or extracellular vesicles purified from mild and severely ill patients (library #3 includes 7 mild cases and 8 severe cases, and library #4 includes 6 mild cases and 9 severe cases).

FIG. 6D shows a box plot representation of the integrative miRNA signature (10 miRNAs) between RNA extracted from EV-CATCHER purified extracellular vesicles and from whole sera between mildly and severely ill patients.

FIG. 6E shows a box plot RT-qPCR validation of the top 4 differentially expressed miRNAs identified between mild and severely ill Covid-19 hospitalized patients by small-RNA sequencing, with RNA extracted from EV-CATCHER purified extracellular vesicles. Data are represented as ΔΔCt with technical triplicates performed for each individual sample. Differential expression was assessed with DESeq2 (R/Bioconductor package) for sequencing results and t-tests for miRNA score and qPCR results.

FIG. 7A shows six convalescent serum samples quantified for presence of immunoglobulins (IgG) targeting the Receptor Binding Domain (RBD) of SARS-CoV-2 spike protein as antigen. Three high IgG titer (left) and three below quantification level (BLQ) IgG titer serum samples quantified by ELISA. High IgG titers were estimated with activity remaining at a 10,000 fold dilution, while BLQ IgG serum samples did not contain detectable IgG against the spike protein of SARS-CoV-2.

FIG. 7B shows Western blot analysis of CD63 purified extracellular vesicles using the EV-CATCHER assay from high IgG (lanes 1-3) and BLQ IgG (lanes 4-6) serum samples using anti-Alix, -CD63, -CD9, and -CD81 antibodies (Abcam).

FIG. 7C shows representative Transmission Electron Microscopy (TEM) images of high IgG (CDI-001, FIG. 7B—Lane 1) and BLQ IgG (CDI-004, FIG. 7B—Lane 6) extracellular vesicles isolated from convalescent sera using the EV-CATCHER assay. Direct magnification was 20,000× and scale bars are for 200 nm.

FIG. 7D shows a nanoparticle size distribution characterized using the Spectradyne nCS1 instrument with a TS400 microfluidic cartridges using EV-CATCHER purified extracellular vesicles from all six donors.

FIG. 7E shows a schematic representation of the experimental procedure used to obtain high-purity convalescent serum extracellular vesicles by ultracentrifugation.

FIG. 7F shows an in vitro assessment of SARS-CoV-2 infection using Vero E6 cells treated with whole sera or purified extracellular vesicles. Healthy Vero E6 cells Control (bar 1, no virus) were subjected to mNG SARS-CoV-2 (bar 2, virus), ultracentrifuged extracellular vesicles (extracellular vesicle controls) with high IgG (bar 3) and BLQ IgG (bar 4), mNG SARS-CoV-2 with High IgG whole sera (bars 5, 7 and 9) or BLQ IgG whole sera (bars 11, 13 and 15), mNG SARS-CoV-2 with CD63+ extracellular vesicles from high IgG sera (bars 6, 8 and 10), or CD63+ extracellular vesicles from BLQ IgG sera (bars 12, 14 and 16), and UNG digest buffer (bar 17) and UNG digest buffer with mNG SARS-CoV-2 (bar 18) for 72 hours. Statistical analysis was performed using one-way ANOVA with Bonferroni post hoc correction. All results are presented as mean±SEM (n=3), and ****=p<0.0001 vs virus.

FIG. 7G shows fluorescent imaging of Vero E6 cells infected with the mNeonGreen SARS-CoV-2 reporter virus. Hoechst 33342 and mNeonGreen was visualized using fluorescent Celigo Cell Imaging. Representative fluorescent images display healthy Vero E6 cells (no virus control), mNG SARS-CoV-2 with high IgG whole serum (high IgG serum), mNG SARS-CoV-2 with CD63+ extracellular vesicles purified from high IgG serum (extracellular vesicle (High IgG serum); Patient #CDI-001), mNG SARS-CoV-2 (virus control), mNG SARS-CoV-2 with BLQ IgG whole serum (BLQ IgG serum; Patient #CDI-004), mNG SARS-CoV-2 with CD63+ extracellular vesicles purified from BLQ serum (extracellular vesicle (BLQ serum)). Scale bars on images was at 500 μM.

FIG. 8 depicts a heat map representation of the top 117 expressed circulating small-RNAs in human serum identified by small-RNA next-generation sequencing (columns 1 and 2). Small-RNA sequencing was performed on RNA extracted from Dynabeads™ magnetic beads incubated in whole serum (columns 3 and 4), magnetic beads incubated with whole serum and treated with UNG (columns 5 and 6), magnetic beads covalently bound to CD63 antibody incubated in whole serum (columns 7 and 8) compared to extracellular vesicles obtained by ultracentrifugation (columns 9 and 10). All experiments were performed in duplicate.

FIGS. 9A-9B shows SDS PAGE analysis of exosome extracts from COVID19 convalescent serum (left, high IgG titer convalescent serum, RBD IgG >10,000; right, BLQ convalescent serum) stained to show total protein and ACE-2. FIG. 9B shows a graph of ACE-2 protein expression normalized to total protein for convalescent serum ((left, high IgG titer convalescent serum, RBD IgG >10,000; right, BLQ convalescent serum), depicting the significant increase in ACE-2 in convalescent plasma from individuals with high IgG titers.

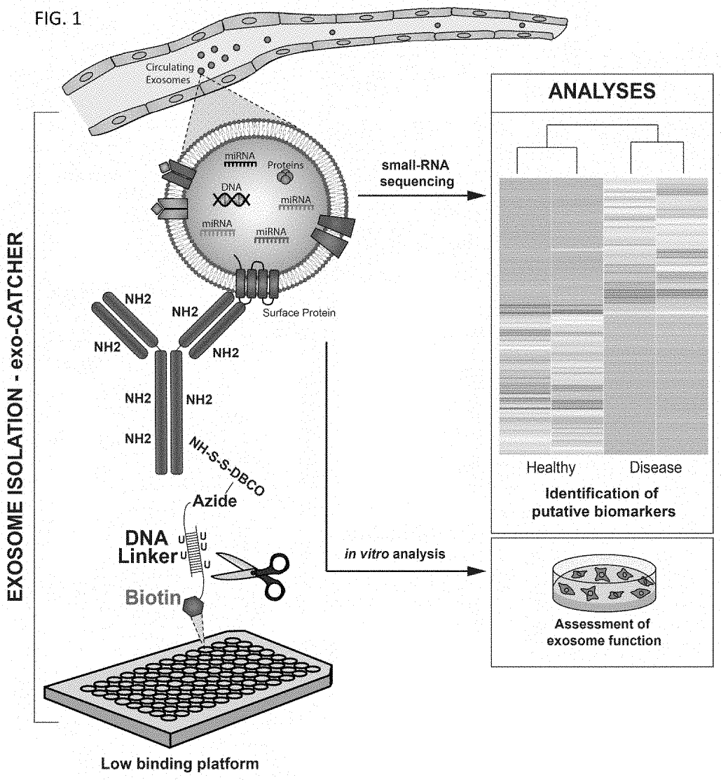

### DETAILED DESCRIPTION OF THE INVENTION

### Definitions

As used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth.

Acute Respiratory Distress Syndrome (ARDS) is characterized by noncardiogenic pulmonary edema, lung inflammation, hypoxemia and decreased lung compliance. The pathogenesis of ARDS remains elusive and there is no gold standard diagnostic test. The Berlin definition of ARDS (Ranieri, V M et al. J. Am. Med. Assoc. (2012) 307 (23): 2526-33) provides the criteria and definitions shown below:

| | Definition |
|---|---|
| Criterion | |
| Timing | Within one week of a known precipitant, or |
| | new/worsening respiratory symptoms |
| Chest imaging | Bilateral opacities - not fully explained by |
| | effusions, lobar/lung collapse, or nodules |
| Origin of edema | Respiratory failure not fully explained by |
| | cardiac failure or fluid overload (if no risk |
| | factor for ARDS is present, need objective |
| | assessment (e.g., echocardiogram) to exclude |
| | hydrostatic edema |
| Oxygenation | |
| Mild | 200 mm Hg < arterial PO2/Fl02 ≤ 300 mg Hg |
| | with PEEP or CPAP ≥ 5 cm H₂O |
| Moderate | 100 mm Hg < arterial PO2/Fl02 ≤ 200 mg Hg |
| | with PEEP or CPAP ≥ 5 cm H₂O |
| Severe | arterial PO2/Fl02 ≤ 100 mg Hg with |
| | PEEP ≥ 5 cm H₂O |

The term “angiotensin-converting enzyme 2” or “ACE2” as used herein refers to a type 1 integral membrane glycoprotein [Tikellils, C. and Thomas M C. Intl J. Peptides (2012) 256294, citing Tipnis, S R et al. J. Biol. Chem. (2000) 275 (43): 33238-43] expressed and active in most tissues that is a component of the renin-angiotensin system (RAS), which controls blood pressure by regulating the volume of fluids in the body. The highest expression of ACE2 is observed in the kidney, the endothelium, the lungs, and the heart [Id., citing Donoghue, M. et al. Cir. Res. (2000) 87 (5): E1-E9, Tipnis, S R et al. J. Biol. Chem. (2000) 275 (43): 33238-43].

As used herein the term “administering”, when used in conjunction with a therapeutic, means to give or apply a therapeutic directly into or onto a target organ, tissue or cell, or to administer a therapeutic to a subject, whereby the therapeutic positively impacts the organ, tissue, cell, or subject to which it is targeted. Thus, as used herein, the term “administering”, when used in conjunction with compositions comprising, for example, an exosome and/or extracellular vesicle, can include, but is not limited to, providing the composition into or onto the target organ, tissue or cell; or providing a composition to a patient so that the therapeutic reaches the target organ, tissue or cell. “Administering” may be accomplished parenterally, e.g., by intravenous administration or infusion techniques, inhalation or insufflation, or by such methods in combination with other known techniques.

The term “Alix” as used herein refers to an accessory protein of endosomal sorting complex required for transport (ESCRT). Evidence indicates that Alix is involved in the packaging of miRNAs during extracellular vesicle (EV) biogenesis [Iavello, A. et al. Int. J. Mol. Med. (2016) 37 (4): 958-66].

The term “angiotensin converting enzyme 2 receptor” or “ACE2 receptor” as used herein refers to a protein that provides the entry point for SARSCoV-2 to infect a wide range of human cells.

The terms “animal,” “patient,” and “subject” as used herein include, but are not limited to, humans and non-human vertebrates such as wild, domestic, and farm animals. The terms “animal,” “patient,” and “subject” may refer to mammals, including, but not limited to, humans.

The term “antibody” as used herein refers to a polypeptide or group of polypeptides comprised of at least one binding domain that is formed from the folding of polypeptide chains having three-dimensional binding spaces with internal surface shapes and charge distributions complementary to the features of an antigenic determinant of an antigen.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface. Human antibodies show two kinds of light chains, κ and λ; individual molecules of immunoglobulin generally are only one or the other.

An antibody may be an oligoclonal antibody, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody, and an antibody that can be labeled in soluble or bound form, as well as fragments, variants or derivatives thereof, either alone or in combination with other amino acid sequences provided by known techniques. Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies. Diverse libraries of immunoglobulin heavy (VH) and light (Vκ and Vλ) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage. The technique of guided selection is based on human immunoglobulin V gene shuffling with rodent immunoglobulin V genes. The method entails (i) shuffling a repertoire of human λ, light chains with the heavy chain variable region (VH) domain of a mouse monoclonal antibody reactive with an antigen of interest; (ii) selecting half-human Fabs on that antigen (iii) using the selected λ, light chain genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1, λ, antibody molecule in the mouse myeloma. An antibody may be from any species. The term antibody also includes binding fragments of the antibodies of the invention; exemplary fragments include Fv, Fab, Fab′, single stranded antibody (svFC), dimeric variable region (Diabody) and di-sulphide stabilized variable region (dsFv). Structural and functional domains can be identified by comparison of the nucleotide and/or amino acid sequence data to public or proprietary sequence databases. For example, computerized comparison methods can be used to identify sequence motifs or predicted protein conformation domains that occur in other proteins of known structure and/or function. Methods to identify protein sequences that fold into a known three-dimensional structure are known. See, for example, Bowie et al. Science 253:164 (1991), which is incorporated by reference in its entirety.

As used herein, the terms “antigen” refers to any substance that elicits an immune response.

The term “antigen-binding site” as used herein refers to the site at the tip of each arm of an antibody that makes physical contact with an antigen and binds it noncovalently. The antigen specificity of the antigen-binding site is determined by its shape and the amino acids present.

The term “antigenic determinant” or “epitope” as used herein refers to that portion of an antigenic molecule that is contacted by the antigen-binding site of a given antibody or antigen receptor.

The term “aptamer” as used herein refers to single stranded synthetic oligonucleotides that fold into three-dimensional shapes capable of binding non-covalently with high affinity and specificity to a target molecule. Rather than primary sequence, binding of an aptamer is determined by its tertiary structure. Target recognition and binding involve three dimensional, shape-dependent interactions as well as hydrophobic interactions, base-stacking, and intercalation.

The term “binding” and its other grammatical forms as used herein means a lasting attraction between chemical substances. Binding specificity involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

As used herein, the term “binding agent” refer to a substance that can bind to a chemical or other substance, e.g., an antigen.

The terms “biodegradable” and “biologically degradable” as used herein refer to a material that will erode to soluble species or that will degrade under physiologic conditions to smaller units or chemical species that are, themselves, non-toxic (biocompatible) to the subject and capable of being metabolized, eliminated, or excreted by the subject.

As used herein, the term “biological particle” refers to a minute portion, piece, fragment or amount (particle) derived from an organism. Biological particles include, without limitation, exosomes, extracellular vesicles, viral particles, bacterial particles, or other secreted particles comprising surface membranes.

The term “biomarker” (or “biosignature”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (e.g., choices of drug treatment or administration regimes).

The term “cargo” as used herein refers to a load or that which is conveyed. With respect to exosomes and/or extracellular vesicles, the term cargo refers to a substance encapsulated in the exosome and/or extracellular vesicle. The compound or substance can be, e.g., a nucleic acid (e.g., nucleotides, DNA, RNA), a polypeptide, a lipid, a protein, or a metabolite, or any other substance that can be encapsulated in an exosome and/or extracellular vesicle. With respect to exosomes and/or extracellular vesicles, the term “cargo profile” as used herein refers to the measurement of the abundance of cargo components (e.g., a nucleic acid (e.g., nucleotides, DNA, RNA), a polypeptide, a lipid, a protein, or a metabolite) that characterize the population of exosomes and/or extracellular vesicles.

The term “CD9” as used herein refers to a member of the tetraspanin protein family whose crystal structure shows a reversed cone-like molecular shape, which generates membrane curvature in the crystalline lipid layers. (Umeda, R. et al. Nature Communic. (2020) 11: article 1606).

The term “CD37” as used herein refers to a member of the tetraspanin protein family exclusively expressed on immune cells. (Zuidscherwoude, M. et al. Scientific Reports (2015) 5: 12201).

The term “CD63” as used herein refers to a member of the tetraspanin protein family, the C-terminal domain of which interacts with several subunits of adaptor protein (AP) complexes, linking the traffic of this tetraspanin to clathrin-dependent pathways (Andreu, Z. & Yanez-Mo, M., citing Rous, B A et al. Mol. Biol. Cell (2002) 13 (3): 1071-82). Among intracellular interacting proteins, CD63 was shown to directly bind to syntenin-1, a double PDZ domain-containing protein (Id., citing Latysheva, N. et al. Mol. Cell Biol. (2006) 26 (20): 7707-18). A major role in exosome biogenesis has been reported for Syntenin-1 (Id., citing Baietti, M F et al. Nat. Cell Biol. (2012) 14 (7): 677-85).

The term “CD81” as used herein refers to a member of the tetraspanin protein family whose crystal structure shows a reversed teepee-like arrangement of the four transmembrane (TM) helices, which create a central pocket in the intramembranous region that appears to bind cholesterol in the central cavity. (Zimmerman, B. et al. Cell (2016) 167: 1041-51). During development, CD81 regulates the trafficking of CD19, an essential co-stimulatory molecule of lymphoid B cells and a well-characterized CD81 partner, along the secretory pathway. (Shoham, T. et al. J. Imunol. (2003) 171: 4062-72). CD9 and CD81 have been shown to regulate several cell-cell fusion processes. (Chaffin, S. et al. J. Cell Science (2014) 127: 3641-48).

The term “CD82” as used herein refers to a member of the tetraspanin protein family that has been implicated in the regulation of protein sorting into EVs and in antigen presentation by antigen presenting cells. (Andreu, Z. and Yanez-Mo, M. Front. Immunol. (2014) doi.org/10.3389/fimmu.2014.00442).

The term “click chemistry” as used herein refers to chemical synthetic methods for making compounds using reagents that can be joined together using efficient reagent conditions and that can be performed in benign solvents or solvents that can be removed or extracted using facile methods, such as evaporation, extraction, or distillation. Several types of reactions that fulfill these criteria have been identified, including nucleophilic ring opening reactions of epoxides and aziridines, non-aldol type carbonyl reactions, such as formation of hydrazones and heterocycles, additions to carbon-carbon multiple bonds, such as oxidative formation of epoxides and Michael additions, and cycloaddition reactions. A representative example of click chemistry is a reaction depicted in Formula I below that couples an azide and an alkyne to form a triazole. The copper-catalyzed azide-alkyne cycloaddition (CuAAC) features an enormous rate acceleration of 10⁷ to 10⁸ compared to the uncatalyzed 1,3-dipolar cycloaddition. It succeeds over a broad temperature range, is insensitive to aqueous conditions and pH range over 4 to 12, and tolerates a broad range of functional groups. Pure products can be isolated by simple filtration or extraction without the need for chromatography or recrystallization.

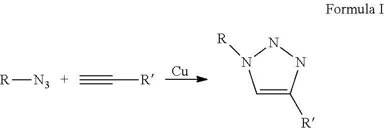

A representative example of copper-free click chemistry is a reaction that couples a dibenzocyclo-octyl (DBCO)-tagged DNA molecule to an azide-functionalized surface by cycloaddition without copper as shown in Formula II:

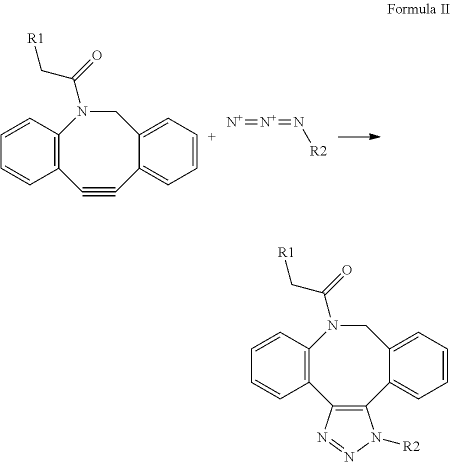

[Eeftens, J M, et al. BMC Biophys. (2015) 8: 9].

The term “clickable functional group” as used herein refers to a functional group that can be used in click chemistry to form a product. According to some embodiments, the clickable functional group is an azide or an alkyne.

The term “conjugate” as used herein refers to a compound formed by the joining or linking together of two or more chemical compounds.

The term “contact” and its various grammatical forms as used herein refers to a state or condition of touching or of immediate or local proximity.

The term “convalescent serum” as used herein refers to blood serum obtained from an individual who has recovered from an infectious disease and that contains antibodies against the infectious agent that caused the disease. The terms “convalescent serum” and “convalescent plasma” may be used interchangeably.

The term “coronavirus” or “CoV” (CoVs, plural) as used herein refers to a large family of single-stranded RNA viruses that can infect a wide variety of animals, causing respiratory, enteric, hepatic and neurological diseases [Yin, Y., Wunderink, R G, Respirology (2018) 23 (2): 130-37, citing Weiss, S R, Leibowitz, I L, Coronavirus pathogenesis. Adv. Virus Res. (2011) 81: 85-164]. Human coronaviruses, which were considered to be relatively harmless respiratory pathogens in the past, have now received worldwide attention as important pathogens in respiratory tract infection. As the largest known RNA viruses, CoVs are further divided into four genera: alpha-, beta-, gamma- and delta-groups; the beta group is further composed of A, B, C and D subgroups. [Xia, S. et al. Sci. Adv. (2019) 5: eaav4580].

CoVs are enveloped with a non-segmented, positive sense, single strand RNA, with size ranging from 26,000 to 37,000 bases; this is the largest known genome among RNA viruses [Yang, Y. et al., J. Autoimmunity (2020) doi.org/10.1016/j.jaut.2020.102434, citing Weiss, S R et al. Microbiol. Mol. Biol. Rev. (2005) 69 (4): 635-64]. The viral RNA encodes structural proteins, and genes interspersed within the structural genes, some of which play important roles in viral pathogenesis [Yang, Y. et al., J. Autoimmunity (2020) doi.org/10.1016/j.jaut.2020.102434, citing Fehr, A R, Perlman, S. Methods Mol. Biol. (2015) 1282: 1-23; Zhao, L. et al. Cell Host Microbe (2012) 11(6): 607-16]. The spike protein (S) is responsible for receptor binding and subsequent viral entry into host cells; it consists of 51 and S2 subunits. The membrane (M) and envelope (E) proteins play important roles in viral assembly; the E protein is required for pathogenesis [Id., citing DeDiego, M L, et al. J. Virol. (2007) 81(4): 1701-13; Nieto-Torres, J L et al. PLoS Pathog. (2014) 10(5): e1004077]. The nucleocapsid (N) protein contains two domains, both of which can bind virus RNA genomes via different mechanisms and is necessary for RNA synthesis and packaging the encapsulated genome into virions. [Yang, Y. et al., J. Autoimmunity (2020) doi.org/10.1016/j.jaut.2020.102434., citing Fehr, A R, Perlman, S. Methods Mol. Biol. (2015) 1282: 1-23; Song, Z. et al. Viruses (2019) 11(1): 59; Chang, C K et al., J. Biomed. Sci. (2006) 13(1): 59-72; Hurst, K R, et al. J. Virol. (2009) 83 (14): 7221-34]. The N protein also is an antagonist of interferon and viral encoded repressor (VSR) of RNA interference (RNAi), which benefits viral replication [Id., citing Cui, L. et al. J. Virol. (2015) 89 (17): 9029-43].

Before December 2019, six coronavirus species had been identified to infect humans and cause disease. Among them, infections caused by H-CoV-229E and HCoV-NL63 in the alpha group, HCoV-OC43 and HCoV-HKU1 in beta subgroup A are frequently mild, mostly causing common cold symptoms [Xu, X. et al. Eur. J. Nuclear Medicine & Molec. Imaging (2020) doi.org/10.1007/s00259-020-04735-9, citing Su, S. et al. Trends Microbiol. (2016) 24: 490-502]. The other two species, severe acute respiratory syndrome coronavirus (SARS-CoV) in beta subgroup B and Middle East respiratory syndrome coronavirus (MERS-CoV) in beta subgroup C, have a different pathogenicity and have caused fatal illness [Id., citing Cui, J. et al. Nat. Rev. Microbiol. (2019) 17: 181-92]. Severe Acute Respiratory Syndrome Coronavirus 2 (SARS-CoV-2) is the seventh member of the coronaviruses that infects humans [Zhu, N. et al. N. Engl. J. Med. (2020) 382: 727-33]. Host cell entry of CoVs depends on binding of the viral spike (S) proteins to cellular receptors and on S protein priming by host cell proteases, which entails S protein cleavage at the S1/S2 and the ST site and allows fusion of viral and cellular membranes, a process driven by the S2 subunit. [Hoffmann, M. et al. Cell (2020) 181 (2): 271-80]. Angiotensin converting enzyme 2 (ACE2) and dipeptidyl peptidase 4 (DPP4) are known host receptors for SARS-CoV and MERS-CoV respectively [Yang, Y. et al., J. Autoimmunity (2020) doi.org/10.1016/j.jaut.2020.102434, citing Kuhn, J H, et al. Cell Mol. Life Sci. (2004) 61 (21): 2738-43; Raj, V S, et al. Nature (2013) 495 (7440): 251-54]. SARSCoV-2 uses the SARS-CoV receptor ACE2 to gain entry into host cells and the serine protease TMPRSS2 for S protein priming. [Hoffman, M. et al. Cell (2020) 181 (2): 271-80]. One mechanism for SARS-CoV-2 entry occurs when the spike protein on the surface of SARS-CoV-2 binds to an ACE2 receptor followed by cleavage at two cut sites (“priming”) that causes a conformational change allowing for viral and host membrane fusion. [Shrimp, J H et al. ACS Pharmacol. Trans. Sci. (2020) 3(5): 997-1007].

As used herein, the term “derived from” refers to any method for receiving, obtaining, or modifying something from a source of origin.

The term “DNA library” as used herein refers to a collection of DNA fragments that have been cloned into vectors so that researchers can identify and isolate the DNA fragments that interest them for further study.

The term “domain” as used herein refers to a region of a protein with a characteristic tertiary structure and function and to any of the three-dimensional subunits of a protein that together make up its tertiary structure formed by folding its linear peptide chain.

The term “encapsulated” as used herein refers to being enclosed in a capsule (meaning a membranous envelope enclosing a part).

The term “Endosomal Sorting Complexes required for transport” (ESCRTs) refers to components involved in multivesicular body (MVB) and intraluminal vesicle (ILV) biogenesis. ESCRTs consist of approximately twenty proteins that assemble into four complexes (ESCRT-0, -I, -II and -III) with associated proteins (VPS4, VTA1, ALIX), which are conserved from yeast to mammals (Colombo, M. et al. J. Cell Science (2013) 126: 5553-65, citing Henne, W. M., et al. (2011). Dev. Cell 21, 77-91; Henne et al., 2011; Roxrud, I. et al. (2010). ESCRT & Co. Biol. Cell 102, 293-318). The ESCRT-0 complex recognizes and sequesters ubiquitylated proteins in the endosomal membrane, whereas the ESCRT-I and -II complexes appear to be responsible for membrane deformation into buds with sequestered cargo, and ESCRT-III components subsequently drive vesicle scission (Id., citing Hurley, J. H. and Hanson, P. I.(2010). Nat. Rev. Mol. Cell Biol. 11, 556-566; Wollert, T. et al. Nature (2009) 458: 172-77). ESCRT-0 comprises hepatocyte growth factor-regulated tyrosine kinase dubstrate (HRS) protein that recognizes the mono-ubiquitylated cargo proteins and associates in a complex with signal-transducing adaptor molecule (STAM), Eps15 and clathrin. HRS recruits TSG101 of the ESCRT-I complex, and ESCRT-I is then involved in the recruitment of ESCRT-III, through ESCRT-II or ALIX, an ESCRT-accessory protein. Finally, the dissociation and recycling of the ESCRT machinery requires interaction with the ATPase associated with various cellular activities (AAA-ATPase) Vps4; Vps4 releases ESCRT-III from the MVB membrane for additional sorting events. It is unclear whether ESCRT-II has a direct role in ILV biogenesis or whether its function is limited to particular cargo (Id., citing Bowers, K. et al. (2006) J. Biol. Chem. 281, 5094-5105; Malerød, L. et al. Traffic 8, 1617-1629).

Concomitant depletion of ESCRT subunits belonging to the four ESCRT complexes does not totally impair the formation of MVBs, indicating that other mechanisms may operate in the formation of ILVs and thereby of exosomes and/or extracellular vesicles (Id., citing Stuffers, S. et al., (2009) Traffic 10, 925-937). One of these pathways requires a type II sphingomyelinase that hydrolyses sphingomyelin to ceramide (Id., citing Trajkovic, K. et al. (2008) Science 319, 1244-1247). Although the depletion of different ESCRT components does not lead to a clear reduction in the formation of MVBs and in the secretion of proteolipid protein (PLP) associated to exosomes and/or extracellular vesicles, silencing of neutral sphingomyelinase expression with siRNA or its activity with the drug GW4869 decreases exosome and/or extracellular vesicle formation and release. However, whether such dependence on ceramides is generalizable to other cell types producing exosomes and/or extracellular vesicles and additional cargos has yet to be determined. The depletion of type II sphingomyelinase in melanoma cells does not impair MVB biogenesis (Id., citing van Niel, G. et al., (2011) Dev. Cell 21, 708-721) or exosome and/or extracellular vesicle secretion, but in these cells the tetraspanin CD63 is required for an ESCRT-independent sorting of the luminal domain of the melanosomal protein PMEL (van Niel et al., (2011) Dev. Cell 21, 708-721). Moreover, tetraspanin-enriched domains have been proposed to function as sorting machineries allowing exosome and/or extracellular vesicle formation (Perez-Hernandez, D. et al. J. Biol. Chem. (2013) 288, 11649-11661).

Despite evidence for ESCRT-independent mechanisms of exosome and/or extracellular vesicle formation, proteomic analyses of purified exosomes and/or extracellular vesicles from various cell types have identified ESCRT components (TSG101, ALIX) and ubiquitylated proteins (Id., citing Buschow, S. et al., (2005) Blood Cells Mol. Dis. 35, 398-403; Théry, C. et al., (2006). Curr. Protoc. Cell Biol. Chapter 3, Unit 3.22). It has also been reported that the ESCRT-0 component HRS could be required for exosome and/or extracellular vesicle formation and/or secretion by dendritic cells (DCs), and thereby impact on their antigen-presenting capacity (Id., citing Tamai, K. et al., (2010) Biochem. Biophys. Res. Commun. 399, 384-390). The transferrin receptor (TfR) in reticulocytes that is generally fated for exosome and/or extracellular vesicle secretion, although not ubiquitylated, interacts with ALIX for MVB sorting (Id., citing Géminard, C. et al., (2004). Traffic 5, 181-193). It was also shown that ALIX is involved in exosome and/or extracellular vesicle biogenesis and exosomal sorting of syndecans (a major family of cell surface heparan sulfate proteoglycans (HSPGs) composed of sulfated glycosaminoglycans (GAGs), heparan sulfate (HS) or both HS and chondroitin sulfate (CS), attached covalently to core proteins—see Park, P W. Methods Cell Biol. (2018) 143: 317-33) through its interaction with syntenin (Colombo, M. et al. J. Cell Science (2013) 126: 5553-65, citing Baietti, M F et al., (2012). Nat. Cell Biol. 14, 677-685). Silencing of genes for two components of ESCRT-0 (HRS, STAM1) and one of ESCRT-I (TSG101), as well as a late acting component (VPS4B) induced consistent alterations in exosome and/or extracellular vesicle secretion. [Colombo, M. et al. J. Cell Sci. (2013) 126: 5553-65].

The term “exosomes and/or extracellular vesicles” as used herein refers to extracellular bilayered membrane-bound vesicles of endosomal origin in a size range of −40 to 160 nm in diameter (˜100 nm on average) generated by all cells that are actively secreted.

Biogenesis. Exosomes and/or extracellular vesicles are generated in a process that involves double invagination of the plasma membrane and the formation of intracellular multivesicular bodies (MVBs) containing intraluminal vesicles (ILVs). ILVs are ultimately secreted as exosomes and/or extracellular vesicles with a size range of ˜40 to 160 nm in diameter through MVB fusion to the plasma membrane and exocytosis. The first invagination of the plasma membrane forms a cup-shaped structure that includes cell-surface proteins and soluble proteins associated with the extracellular milieu. This leads to the de novo formation of an early-sorting endosome (ESE) and in some cases may directly merge with a preexisting ESE. The trans-Golgi network and endoplasmic reticulum can also contribute to the formation and the content of the ESE (Kalluri, R., LeBleu, VS. Science (2020) 367 (6478): eaau6977, citing Kalluri, R. J. Clin. Invest. (2016) 126: 1208-15; van Neil, G. et al. Nat. Rev. Mol. Cell Biol. (2018) 19: 213-28; McAndrews, KM, Kalluri, R. Mol. Cancer (2019) 18: 52; Mathieu, M. et al. Nat. Cell Biol. (2019) 21: 9-17; Willms, E. et al. Front. Immunol. (2018) 9: 738; Hessvik, N P, Llorente, A. Cell Mol. Life Sci. (2018) 75: 193-208). ESEs can mature into late-sorting endosomes (LSEs) and eventually generate MVBs, which are also called multivesicular endosomes. MVBs form by inward invagination of the endosomal limiting membrane (that is, double invagination of the plasma membrane). This process results in MVBs containing several ILVs (future exosomes and/or extracellular vesicles). The MVB can either fuse with lysosomes or autophagosomes to be degraded or fuse with the plasma membrane to release the contained ILVs as exosomes and/or extracellular vesicles [Id., citing Kahler, C., Kalluri, R. J. Mol. Med. (2013) 91: 431037].

Heterogeneity: The heterogeneity of extracellular vesicles is thought to be reflective of their size, content, functional impact on recipient cells, and cellular origin. During their secretion they acquire surface proteins from their cell of origin. They naturally transport mRNA, miRNA, and proteins between cells.

Biomarkers. There is general agreement that their membranes are specifically enriched in tetraspanins CD9, CD37, CD63, CD81, and CD82.

Role. Extracellular vesicles are mediators of near and long-distance intercellular communication in health and disease and affect various aspects of cell biology.

The term “extracellular vesicles (EVs)” as used herein refers to nanosized, membrane-bound vesicles released from cells that can transport cargo—including DNA, RNA, and proteins—between cells as a form of intercellular communication. Different EV types, including microvesicles (MVs), exosomes, oncosomes, and apoptotic bodies, have been characterized on the basis of their biogenesis or release pathways. Microvesicles bud directly from the plasma membrane, are 100 nanometers (nm) to 1 micrometer (μm) in size, and contain cytoplasmic cargo (Zaborowski, M P et al. BioScience (2015) 65 (8): 783-97, citing Heijnen, H F et al. Blood (1999) 94: 3791-99). Another EV subtype, exosomes, is formed by the fusion between multivesicular bodies and the plasma membrane, by which multivesicular bodies release smaller vesicles (exosomes) whose diameters range from 40 to 160 nm (Id., citing El Andaloussi, S. et al. Nature Reviews Drug Discovery (2013) 12: 347-57; Cocucci, E. and Meldolesi J. Trends in Cell Biology (2015) 25: 364-72). Dying cells release vesicular apoptotic bodies (50 nm-2 μm) that can be more abundant than exosomes or MVs under specific conditions and can vary in content between biofluids (Id., citing Thery, C. et al J Immunology (2001) 1666: 7309-18; El Andaloussi, S. et al. Nature Reviews Drug Discovery (2013) 12: 347-57). Membrane protrusions can also give rise to large EVs, termed oncosomes (1-10 μm), which are produced primarily by malignant cells in contrast to their nontransformed counterparts (Id., citing Di Vizio, D. et al. Am. J. Pathol. (2012) 181: 1573-84; Morello, M. et al. Cell Cycle (2013) 12: 3526-36).

The term “inhibitor” as used herein refers to a molecule that reduces the amount or rate of a process, stops the process entirely, or that decreases, limits, or blocks the action or function thereof. Enzyme inhibitors are molecules that bind to enzymes thereby decreasing enzyme activity. Inhibitors may be evaluated by their specificity and potency. Inhibition may include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%.

The term “isolated” is used herein to refer to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to more than about 95%, 96%, 97%, 98%, 99% or 100% free. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state.

The term “Fab fragment” as used herein refers to an antibody fragment composed of a single antigen-binding arm of an antibody without the Fc region, produced by cleavage of IgG by the enzyme papain. It contains the complete light chain plus the amino-terminal variable region and first constant region of the heavy chain, held together by an interchain disulfide bond.

The term “F(ab′)₂ fragment as used herein refers to an antibody fragment composed of two linked antigen-binding arms (Fab fragments) without the Fc regions, produced by cleavage of IgG with pepsin.

The term “fragment” or “peptide fragment” as used herein refers to a small part derived, cut off, or broken from a larger peptide, polypeptide or protein, which retains the desired biological activity of the larger peptide, polypeptide or protein. Antibody binding fragments (e.g., Fab, Fab′, F(ab′)₂, Fv, and single-chain (sc) antibodies) can be produced by recombinant DNA techniques, or by enzymatic or chemical cleavage of intact antibodies.

The term “heterogeneous” as used herein refers to being composed of unrelated or unlike elements or parts; varied; miscellaneous; of different kinds; differing or opposite in structure, quality etc.; dissimilar.

The term “high throughput screening” or “HTS” as used herein refers to the use of automated equipment to rapidly test thousands to millions of samples for biological activity at the model organism, cellular, pathway, or molecular level.

The term “homogeneous” as used herein refers to being of the same character, structure, quality; etc.; essentially like; of the same nature; composed of similar or identical elements or parts; uniform.

The terms “immune response” and “immune-mediated” are used interchangeably herein to refer to any functional expression of a subject's immune system, against either foreign or self-antigens, whether the consequences of these reactions are beneficial or harmful to the subject.

The term “immunomodulatory” as used herein refers to a substance or agent that is capable of augmenting or diminishing immune responses directly or indirectly, e.g., by expressing chemokines, cytokines and other mediators of immune responses.

The term “inhalation” as used herein refers to the act of drawing in a medicated vapor with the breath.

The term “inhalation delivery device” as used herein refers to a machine/apparatus or component that produces small droplets or an aerosol from a liquid or dry powder aerosol formulation and is used for administration through the mouth in order to achieve pulmonary administration of a drug, e.g., in solution, in suspension, as a powder, and the like.

The term “join” as used herein means to link, couple, or connect one thing with another. Each of these terms is used interchangeably with the others.

The term “long noncoding RNA” (“lncRNAs”) as used herein refers to a class of transcribed RNA molecules that are longer than 200 nucleotides and yet do not encode proteins. LncRNAs can fold into complex structures and interact with proteins, DNA and other RNAs, modulating the activity, DNA targets or partners of multiprotein complexes. Crosstalk of lncRNAs with miRNAs creates an intricate network that exerts post-transcriptional regulation of gene expression. For example, lncRNAs can harbor miRNA binding sites and act as molecular decoys or sponges that sequester miRNAs away from other transcripts. Competition between lncRNAs and miRNAs for binding to target mRNAs has been reported and leads to de-repression of gene expression (Zampetaki, A. et al. Front. Physiol. (2018) doi.org/10.3389/fphys.2018.01201, citing Yoon, J H et al. Semin Cell Dev. Bio. (2014) 34: 9-14; Ballantyne, M D et al. Clin. Pharmacol. Ther. (2016) 99: 494-501). Finally, lncRNAs may contain embedded miRNA sequences and serve as a source of miRNAs (Id., citing Piccoli, M T et al. Cir. Res. (2017) 121: 575-83).

The term “messenger RNA” (“mRNA”) as used herein refers to a coding RNA, which functions in protein translation.

The term “microRNA” (or “miRNA”) as used herein refers to a class of small, 18- to 28-nucleotide-long, noncoding RNA molecules. Their major role is in the posttranscriptional regulation of protein expression

As used herein, the term “neutralization” and its other grammatical forms is used to refer to inhibition of the infectivity of a virus, for example, by inhibiting the viral particles from attaching and entering into a host cell, which effectively inhibits the viral replication cycle. The term “neutralization titer” as used herein refers to a minimum amount or concentration of a substance derived from a biological sample necessary to cause neutralization.

The terms “next generation sequencing”, “NGS”, “massively parallel sequencing”, or “deep sequencing” as used herein describe a high-throughput method used to determine the nucleotide sequence of an individual's whole genome at once in an automated process. First a DNA library is prepared from a patient's sample by fragmentation, purification and amplification of the DNA sample. Individual fragments are then physically isolated by attachment to solid surfaces or small beads. The sequence of each of these fragments is resolved simultaneously by such techniques as sequencing by synthesis. The resulting sequence data are computationally aligned against a ‘normal reference’ genome.

The term “non-coding RNA” (“ncRNA”) as used herein refers to a functional RNA molecule that is transcribed from DNA but not translated into proteins. They are classified into housekeeping and regulatory noncoding RNAs. Housekeeping ncRNAs include ribosomal RNA (rRNA, the RNA component of ribosomes), transfer RNA (tRNA, which functions as an adapter for matching amino acids to mRNA), small nuclear RNA (snRNA, which functions in RNA processing such as mRNA splicing), and small nucleolar RNAs (snoRNAs, which functions in guiding chemical modification of other RNAs). Regulatory noncoding RNAs are divided into short ncRNAs (<200 nt) and long ncRNAs (>200 nts). Short noncoding RNAs <200 nt include microRNA (miRNA), small interfering RNAs (siRNAs) and piwi-associated RNAs (piRNAs), and long noncoding RNAs (>200 nt). [Losko, M. et al. Mediators of Inflammation (2016) 1-12. 10.1155/2016/5365209].

The term “nucleic acid” as used herein refers to a deoxyribonucleotide or ribonucleotide polymer in either single- or double-stranded form, and, unless otherwise limited, encompasses known analogues having the essential nature of natural nucleotides in that they hybridize to single-stranded nucleic acids in a manner similar to naturally occurring nucleotides (e.g., peptide nucleic acids).

The term “nucleotide” as used herein refers to a molecule consisting of a nitrogen-containing base (adenine, guanine, thymine, or cytosine in DNA; adenine, guanine, uracil, or cytosine in RNA), a phosphate group, and a sugar (deoxyribose in DNA; ribose in RNA).

The term “particle” as used herein refers to an extremely small constituent, e.g., nanoparticles.

The term “PDZ domain” as used herein refers to abundant protein interaction modules that often recognize short amino acid motifs at the C-termini of target proteins. They have been implicated in regulating multiple biological processes, such as transport, ion channel signaling and other signal transduction systems. (see Lee, H-J & Zheng, J J. Cell Communication & Signaling (2010) 8: article 8).

The term “peptidominetic” as used herein refers to a small protein-like chain or non-peptidic structural elements capable of mimicking (meaning imitating) or antagonizing (meaning neutralizing or counteracting) the biological action(s) of a natural parent peptide.

The term “placental alkaline phosphatase” or “PLAP” refers to an enzyme normally produced by primordial germ cells and syncytiotrophoblasts; the detection of its expression has been useful in the diagnosis of germ cell tumors. PLAP immunoreactivity in normal human adult and fetal muscle tissue has been observed. This immunoreactivity seems to relate to the degree of myogenic differentiation in soft tissue tumors and is more frequently expressed in cells with skeletal muscle differentiation and least in those with myofibroblastic features. (Goldsmith, J D, et al. Am J. Surgical Pathol (2002) 26 (12): 1627-33).

The term “plasma” as used herein refers to the fluid (noncellular) portion of circulating blood, and the fluid portion of lymph.

The term “polymer” refers to a large molecule, or macromolecule, composed of many repeated subunits. The term “monomer” refers to a molecule that may bind chemically to other molecules to form a polymer. The term “copolymer” as used herein refers to a polymer derived from more than one species of monomer.

The terms “polypeptide” and “protein” are used herein in their broadest sense to refer to a sequence of subunit amino acids, amino acid analogs, or peptidomimetics. The subunits are linked by peptide bonds, except where noted. These terms also apply to amino acid polymers in which one or more amino acid residue is an artificial chemical analogue of a corresponding naturally occurring amino acid, as well as to naturally occurring amino acid polymers. The terms also are inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides may not be entirely linear. For instance, polypeptides may be branched as a result of ubiquitination, or they may be circular, with or without branching, generally as a result of posttranslational events, whether by natural processing or by events brought about by human manipulation, which do not occur naturally. Circular, branched and branched circular polypeptides may be synthesized by entirely synthetic methods.

The term “purification” and its various grammatical forms as used herein refers to a process of isolating or freeing from foreign, extraneous, or objectionable elements. The composition is nonetheless substantially pure in that it has been substantially separated from the substances with which it may be associated in living systems or during synthesis. As used herein, the term “substantially pure” refers purity of at least 75%, at least 80%, at least 85%, at least 90%, at least 95% or at least 99% pure as determined by an analytical protocol. Such protocols may include, for example, without limitation, flow cytometry, electrophoresis, small-RNA sequencing, quantitative PCR, nanoparticle tracking, electron microscopy, mass spectrometry, Western blotting, ELISA, and various metabolic assays.

The term “quantitative PCR” (or “qPCR”), also called “real time-PCR” or “quantitative real-time PCR” refers to a polymerase chain reaction-based technique that couples amplification of a target DNA sequence with quantification of the concentration of that DNA species in the reaction.

The term “receptor binding domain” or “RBD” as used herein refers to a domain on a viral protein that allows a viral protein to bind and attach to host cell receptors and thereby gain entry into and infect host cells. The RBD of SARS-CoV-2 corresponds to residues 331-524 of the S protein. [Tai, W. et al. Cellular & Molecular Immunol. (2020) 17: 613-20].

The renin angiotensin system (RAS) is a central regulator of renal and cardiovascular function. Classically, it consists of angiotensin converting enzyme (ACE), its product, angiotensin (Ang) II and receptors for Ang II, angiotensin Type 1 (AT1) and angiotensin type 2 (AT2) receptors. RAS further includes ACE2, a monocarboxypeptidase that generates Ang-(1-7) from Ang II. Angiotensin-(1-7) is an endogenous ligand for the G protein-coupled receptor Mas; Mas therefore mediates the biological actions of Ang-(1-7) [Singh, N. et al. Am J. Physiol. Heart Circ. Hysiol. (2015) 309 (10): H1697-H1707, citing Santos, R A et al. Proc. Nat. Acad. Sci. USA (2003) 100: 8258-63]. Ang II produces hypertensive, pro-oxidative, hypertrophic and pro-fibrotic effects in the cardiovascular system. Ang-(1-7) elicits counter-regulatory effects on the ACE/AngII pathway by reducing vasodilatory, antihypertensive, antihypertrophic, antifibrotic and antithrombotic effects [Id., citing Ferreira, A J, et al. Hypertension (2010) 55: 207-13; Jusuf, D. et al. Eur. J. Pharmacol. (2008) 585: 303-12].

The term “restriction enzyme” as used herein refers to an enzyme produced by bacteria that recognizes a short sequence in a nucleic acid molecule and cleaves the nucleic acid molecule at or near that sequence, resulting in a double-stranded or single-stranded break. The term “restriction enzyme recognition site” or “restriction site” as used herein is a short specific sequence of base pairs of DNA recognized by each restriction enzyme.

The term “serum” as used herein refers to the fluid portion of the blood obtained after removal of the fibrin clot and blood cells.

The term “static system” as used herein refers to one without feedback systems whose output at an instant in time depends only on the input at that time. It is distinguished from a “dynamic system”, whose output depends on past and future values of the data at any instant of time.

The term “strand displacement” as used herein refers to a reaction in DNA homologous recombination and DNA mismatch repair. It is a reaction where one of the strands in a double-stranded DNA is replaced with another nearly identical strand. DNA strand displacement involves three single strands named the “invader”, the “incumbent” and the “substrate strands. Basically, it is a swapping reaction between the invader and the incumbent strands on the substrate strand. A double-stranded nucleic acid molecule is separated, partially or completely into two separate single-stranded nucleic acid strands by an enzyme having strand displacement activity (e.g., unwinding activity), such as a topoisomerase or helicase, and an invader strand complementary to the incumbent strand in the double-stranded nucleic acid. The enzyme unwinds the double-stranded nucleic acid allowing for the invader complementary strand to hybridize to one of the separated strands from the double-stranded nucleic acid, thereby blocking the incumbent double-stranded nucleic acid strand from reannealing with its matching substrate strand in the original double stranded DNA molecule. The incumbent single stranded DNA that was displaced then undergoes branch-migration until the incumbent strand is fully displaced by the invader strand. (Broadwater, Jr., D W B, Kim, HD, Biophys. J. (2016) 110 (7): 1476-84). The described and claimed assay comprises DNA displacement, whereby a small oligonucleotide hybridizes to and overhang at the end of the double stranded DNA duplex, onto the DNA strand attached to the antibody and a DNA polymerase reaction allows the initial DNA strands (one connected to the antibody and one connected to the platform via the biotin) to separate so a unique antibody with a unique DNA strand may be released specifically from a group of different antibodies targeting different surface markers of exosomes.

The term “tetraspanin” as used herein refers to membrane-spanning proteins with a conserved structure that function primarily as membrane protein organizers. Members of the tetraspanin family of proteins have four transmembrane domains, which contribute to the creation of a small (EC1) and large (EC2) extracellular loop [Termini, C M, Gillette, J M, Front. Cell Dev. Biol. (2017) 5: 34, citing Abe, M. et al. Cancer Lett. (2008) 266: 163-70). The large extracellular loop contains a conserved Cys-Cys-Gly amino acid motif (CCG-motif), as well as two other conserved cysteine residues. Many members of the tetraspanin family also contain post-translational modifications.

The term “therapeutic agent” as used herein refers to a drug, molecule, nucleic acid, protein, metabolite, cell, composition or other substance that provides a therapeutic effect. The term “active” as used herein refers to the ingredient, component or constituent of the compositions of the described invention responsible for the intended therapeutic effect. The terms “therapeutic agent” and “active agent” are used interchangeably herein.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50 which describes the dose in a particular dosage that is therapeutically effective for a particular disease manifestation in 50% of a population.

The terms “therapeutic amount”, an “amount effective”, or “pharmaceutically effective amount” of an active agent are used interchangeably to refer to an amount that is sufficient to provide the intended benefit of treatment. However, dosage levels are based on a variety of factors, including the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount”, “and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

The term “therapeutic effect” as used herein refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein, the therapeutically effective amount initially may be determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan.

General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

In most clinical situations, drugs are administered in a series of repetitive doses or as a continuous infusion to maintain a steady-state concentration of drug associated with the therapeutic window. To maintain the chosen steady-state or target concentration (“maintenance dose”), the rate of drug administration is adjusted such that the rate of input equals the rate of loss. If the clinician chooses the desired concentration of drug in plasma and knows the clearance and bioavailability for that drug in a particular patient, the appropriate dose and dosing interval can be calculated. However, living cellular therapies break this concept, since they divide and may even take up permanent residence in the body in the case of autologous cellular therapy. Hence what is initially administered can bear little correlation to what is present in the recipient over time.

The term “TCID50” or “Median Tissue Culture Infectious dose” as used herein refers to the concentration at which 50% of the cells are infected when a surface upon which cells have been cultured is inoculated with a diluted solution of viral fluid.

The term “T-cell immunoglobulin and mucin domain containing 4” or “Tim-4” as used herein refers to a phosphatidylserine receptor selectively expressed on antigen presenting cells but not on T cells, and can modulate T cell proliferation. (Liu, W. et al. Front. Immunol. (2020) doi.org/10.3389/fimmu.2020.00537).

The term “titer” as used herein is a measure of the concentration and strength of a substance in a biological sample, such as the concentration of an antibody in blood. Titer is estimated by the highest dilution that still allows a detectable effect. For example, when 1:100 gives a positive test and greater dilutions are negative, the titer is 100.

The terms “treat,” “treated,” or “treating” as used herein refers to both therapeutic treatment and/or prophylactic or preventative measures, wherein the object is to prevent or slow down (lessen) an undesired physiological condition, disorder or disease, or to obtain beneficial or desired clinical results. For the purposes of the present disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without unacceptable side effects. Treatment also includes prolonging survival as compared to expected survival if not receiving treatment.

The term “viral load” as used herein refers to a measurement of the amount of a virus in an organism, typically in the bloodstream, usually stated in virus particles per milliliter.

### Methods

According to one aspect, the present disclosure provides a method for in vitro evaluation of a subject's risk of developing and for treating a severe viral infection comprising:

a) preparing a purified population of biological particles comprising a cargo by:
(1) obtaining a biological sample comprising biological particles from the subject;
(2) contacting the biological sample comprising biological particles from the subject with a binding agent directed to one or more biological particle surface antigens; wherein the binding agent is linked to a nucleic acid, and wherein the nucleic acid is immobilized on a solid support;

b) isolating the biological particles bound by the binding agent from the biological sample;

c) releasing the biological particles bound to the binding agent;

(d) eluting the bound biological particles from the binding agent to form a population of free purified biological particles; and

(e) evaluating the cargo of the purified population of biological particles;

(f) determining risk of the patient for the severe viral infection based on a cargo profile of the purified population of biological particles; and

(g) implementing a therapy appropriate for patients at risk for the severe viral infection.

According to some embodiments, the cargo includes one or more of encapsulated miRNAs and/or small non-coding RNAs, encapsulated polypeptides, encapsulated DNA, surface polypeptides, or surface lipids). According to some embodiments, evaluating the cargo (e.g., comprises: (i) identifying and quantifying the cargo by measuring expression of one or more miRNAs or small-non-coding RNAs encapsulated by the purified biological particles; polypeptides encapsulated by the purified biological particles; polypeptides on the surface of the purified biological particles; DNA encapsulated by the purified biological particles; and lipid of the purified biological particles. According to some embodiments, evaluating the cargo comprises determining a cargo profile for the purified biological particles and comparing the cargo profile for the purified biological particles from the subject to a cargo profile from control subjects (1) not infected with the coronavirus; (2) infected with the coronavirus who developed mild disease; and (3) infected with the coronavirus who developed severe disease. According to some embodiments, evaluating the cargo comprises

(i) extracting RNA from the purified population of biological particles;

(ii) identifying and quantifying expression of one or more small noncoding RNAs comprising microRNAs (miRNAs) encapsulated by the purified population of biological particles; and

(iii) comparing expression of the identified and quantified small noncoding RNAs comprising miRNAs to small noncoding RNAs comprising miRNAs identified and quantified from control subjects i) not infected with the virus; ii) infected with the virus (confirmed by PCR) and who developed mild disease (meaning hospitalized but did not require mechanical ventilation); and iii) infected with the virus (confirmed by PCR) who developed severe disease (meaning patients who displayed Acute Respiratory Distress Syndrome (ARDS, following Berlin classification standards and required mechanical ventilation).

According to some embodiments, the cargo profile of the purified biological particles comprises identified and quantified small noncoding RNAs comprising miRNAs from purified biological particles. According to some embodiments, the cargo profile from the purified biological particles from the subject is compared to the cargo profile from control subjects i) not infected with the virus; ii) infected with the virus (confirmed by PCR) and who developed mild disease (meaning hospitalized but did not require mechanical ventilation); and iii) infected with the virus (confirmed by PCR) who developed severe disease (meaning patients who displayed Acute Respiratory Distress Syndrome (ARDS, following Berlin classification standards and required mechanical ventilation).

According to some embodiments, the determining a cargo profile for the purified biological particles by evaluating cargo of the purified population of biological particles step is by: (i) extracting RNA from the purified population of biological particles; (ii) identifying and quantifying expression of small non-coding RNAs comprising one or more microRNAs (miRNAs) encapsulated by the purified population of exosomes; and (iii) comparing the cargo profile for the purified biological particle to a cargo profile from a control subject (1) not infected with the coronavirus; (2) infected with the coronavirus who developed mild disease; and (3) infected with the coronavirus who developed severe disease;

According to some embodiments, the method comprises an initial ultrafiltration step, an ultracentrifugation step or both to provide a pooled heterogeneous population of biological particles.

According to some embodiments, the severe viral infection is a coronavirus infection. According to some embodiments, the coronavirus infection is a SARS-CoV-1 infection. According to some embodiments, the coronavirus infection is a MERSCoV infection. According to some embodiments, the coronavirus infection is a SARS-CoV-2 viral infection.

According to some embodiments, the biological sample comprises a body fluid. Examples of body fluid include, without limitation, blood, urine, semen (seminal fluid), vaginal secretions, cerebrospinal fluid (CSF), synovial fluid, pleural fluid (pleural lavage), pericardial fluid, peritoneal fluid, amniotic fluid, saliva, nasal fluid, otic fluid, gastric fluid, and breast milk, feces, sweat, tears, nipple aspirates, or other secreted biological fluids. According to some embodiments, the body fluid is a circulating/secreted body fluid, e.g., blood (whole blood, serum, or plasma), CSF, or lymph. According to some embodiments, the biological sample comprises a body tissue (e.g., diseased tissue).

According to some embodiments, the method is a high-throughput assay, which allows for the purification of multiple samples at one, i.e., compatible with 24-, 96-, -384 well plate formats or any other multiwall format. According to some embodiments, the biological fluid from which biological particles comprising a specific biomarker(s) are selected can be reused, e.g., for subsequent rounds to select other biomarkers, to select small noncoding RNAs comprising miRNAs, to isolate RNA, DNA, protein, lipids or a combination thereof, etc., without negatively affecting the biological fluid.

According to some embodiments, the binding agent includes a nucleic acid (e.g., DNA, RNA, or a combination thereof), a polypeptide (e.g., an antibody, an antibody fragment, an aptamer polypeptide, or combination thereof), or a biologically degradable polymer.

According to some embodiments, the binding agent is immobilized directly on a solid support. According to some embodiments, the binding agent is immobilized indirectly on a solid support. According to some such embodiments, the binding agent is coupled to a nucleic acid, which is immobilized on a solid support. According to some embodiments, the nucleic acid is immobilized on a solid support by click chemistry.

According to some embodiments, the binding agent is an antibody specific for a biological particle antigen. According to some embodiments, the biological particle antigen comprises one or more of CD9, CD63, CD81, CD37, CD82, Alix, ACE-2, Tim4, PLAP and/or other cell surface markers inherited by the biological particles. In this way the purification process can be tailored to select specific populations of biological particles as desired, e.g., infected by a virus, derived from a cancer cell, derived from an organ, and scan their content. According to some embodiments, the method includes additional cell-specific antibodies for the biological particles sub-population enrichment to detect low abundance biomarkers. According to some embodiments, the method includes larger biofluid material inputs than tested herein. According to some embodiments, the method includes a pre-amplification step to improve the qPCR detection threshold. According to some embodiments, the method includes additional cell-specific antibodies for biological particles sub-population enrichment to detect low abundance biomarkers, larger biofluid material inputs, and a pre-amplification step to improve the qPCR detection threshold.

According to some embodiments, the antibody specific for the biological particles antigen is activated with a dibenzocyclo-octyl (DBCO)-ester, 2-IT (2-iminothiolane), MBS (3-maleimidobenzoic acid N-hydroxysuccinimide ester), SPDP (N-succinimidyl 3-(2-pyridyldithio) propionate), SATA (N-succinimidyl S-acetylthioacetate), SMCC (succinimidyl 4-(N-maleimidomethyl) cyclohexane-1-carboxylate), Sulfo-SMCC, or derivatives thereof. According to some embodiments, the antibody specific for the biological particles antigen is activated with a dibenzocyclo-octyl (DBCO)-ester. According to some embodiments, the DBCO-modified antibody is coupled to a DNA linker by click chemistry. According to some embodiments, the antibody-DNA linker conjugates are bound to streptavidin coated well plates. According to some embodiments, the well plates are pretreated with RNAse A.

According to some embodiments, the isolating/separating step comprises washing, thereby separating unbound materials in the biological sample from the biological particles bound to the binding agent. According to some embodiments, the biological particles bound to the binding agent are released from the solid support enzymatically. According to some embodiments, the releasing includes enzymatically cleaving the connection between the one or more binding agent and the solid support. According to some embodiments, the purified population of biological particles is released by enzymatic cleavage of the nucleic acid, which is connected to the binding agent. According to some embodiments, the nucleic acid is DNA. According to some embodiments, the DNA comprises one or more ribonucleic acid nucleotide. According to some embodiments the one or more ribonucleic acid nucleotide is uracil, a restriction site sequence, or a RNA DNA duplex. According to some embodiments the enzyme specifically cleaves the nucleic acid at a ribonucleic acid nucleotide or at a specific restriction site sequence, or a specific RNA or DNA strand when RNA and DNA strands are duplexed. According to some embodiments, the ribonucleic acid nucleotide is a uracil nucleotide. According to some embodiments, the enzyme that cleaves the nucleic acid at a uracil nucleotide is uracil glycosylase. According to some embodiments, the enzyme that cleaves at a specific nucleic acid sequence is a restriction enzyme. According to some embodiments, the enzyme that cleaves a RNA/DNA duplex specifically is a restriction endonuclease or RNase-H.

According to some embodiments, the purified population of biological particles is released by strand displacement. According to some embodiments, a first incumbent strand of the nucleic acid is separated from the second substrate strand of the nucleic acid by a complementary invader strand to the first or second strand of the nucleic acid and an enzyme comprising strand displacement activity. According to some embodiments, the enzyme comprising strand displacement activity is DNA polymerase, topoisomerase, or helicase. According to some embodiments, a polymerase chain reaction using an oligonucleotide that is complementary to the region of the DNA attached to the antibody is used to separate the annealed DNA strands to allow release of the antibody from the platform without damaging the DNA strand attached to the antibody.

According to some embodiments, the biological particles are eluted from the binding agent. For example, the biological particles may be eluted by contacting the bound binding agent-biological particle complex with free ligand specific for the binding agent to release the purified population of biological particles.

According to some embodiments, the purified population of biological particles comprise biological particle surface protein biomarkers. According to some embodiments, the biological particle surface protein biomarkers include one or more tetraspanin (e.g., CD9, CD63, CD81, CD37, CD82), Alix, ACE-2, Tim4, PLAP and/or other cell surface markers inherited by the biological particles. According to some embodiments the purified population of biological particles is at least 50% pure, at least 55% pure, at least 60% pure, at least 65% pure, at least 70% pure, at least 75% pure, at least 80% pure, at least 85% pure, at least 90% pure, at least 91%, 92%, 93%, 94%, 95%, 96%, 97%, 98% 99% or 100% pure. According to some embodiments, the purified population of biological particles is homogeneous.

According to some embodiments, RNA species, including small noncoding RNA comprising microRNA (miRNA) or DNA encapsulated in the purified biological particles can be isolated, identified, and quantified. According to some embodiments, the small noncoding RNA comprising miRNA, or DNA can be isolated, identified, and quantified via next generation sequencing and displayed by a heat map. Briefly, next generation sequencing is a high-throughput method used to determine the nucleotide sequence of an individual's whole genome at once in an automated process. Small non-coding RNAs comprising miRNA or DNA identified through sequencing are then displayed using a representation of the data in the form of a map or diagram in which data values are represented as colors (“heat map”) generated after statistical analysis.

According to some embodiments, the small noncoding RNA comprising miRNA content of the isolated biological particles is compared to control subjects by next generation sequencing and heat map generation, i.e., a normal healthy subject with no evidence of infection with the virus; a control subject infected with the virus who developed mild symptoms, and a control subject infected with the virus who developed severe symptoms. According to some embodiments, 10 miRNAs are differentially expressed between mild and severe sample groups of SARS-CoV2 patients using total RNA from serum biological particle: hsa-miR-146a; hsa-miR-126-3p; hsa-miR-424; hsa-miR-151-3p; hsa-miR-126-5p; hsa-miR-627-5p; hsa-miR-145; hsa-miR-205; and hsa-miR-200c. According to some embodiments, miRNA markers of severe COVID disease using total RNA from whole serum include hsa-miR-550-5p, or hsa-miR-629 or both.

According to some embodiments, a fold-change difference (ranging from about 0.75 (decrease) and about 1.5 (increase), inclusive) in the cargo profile is evaluated statistically and identified based on p-values <0.05 to determine risk of the patient for the severe viral infection. According to some embodiments, the small noncoding RNA or protein cargo profile for the purified biological particle is about 1.5-fold lower or 1.5-fold higher, than the cargo profile from the control subject infected with the coronavirus who developed severe disease.

According to some embodiments, the therapy comprises a supportive therapy, such as the intravenous administration of fluids, supplemental oxygen, vapor administered via a nebulizer, etc. According to some embodiments, the therapy comprises a pharmaceutical composition comprising an anti-viral agent, an immunomodulatory agent or both.

According to some embodiments, the anti-viral agent inhibits viral entry, decreases viral load, or both. According to some embodiments, the anti-viral agent is selected from acyclovir, gancidovir, foscarnet; ribavirin; amantadine, azidodeoxythymidine/zidovudine), nevirapine, a tetrahydroimidazobenzodiazepinone (TIBO) compound; efavirenz; remdecivir, lopinavir/ritonavir, umifenovir, favipiravir, ivermectin, and delavirdine.

According to some embodiments, the immunomodulatory agent is a glucocorticoid, or a recombinant interferon. According to some embodiments, the glucocorticoid is a corticosteroid, e.g., prednisone, dexamethasone, azathioprine, mycophenolate, mycophenolate mofetil, or a combination thereof.

According to another aspect, the present disclosure provides a method for enhancing therapeutic effectiveness of convalescent plasma therapy in a patient at risk for a severe SARSCoV-2 infection (COVID) comprising:

a) preparing a purified population of biological particles from convalescent plasma of a convalescent subject by:
(1) obtaining a convalescent serum comprising a high anti-SARSCoV-2 IgG titer from a convalescent subject;
(2) contacting the convalescent serum with a binding agent directed to one or more biological particle surface antigens; wherein the binding agent is linked to a nucleic acid, and wherein the nucleic acid is immobilized on a solid support;

b) isolating the biological particles bound by the binding agent from the biological sample;

c) releasing the biological particles bound to the binding agent of (b);

(d) eluting the bound biological particles from the binding agent to form a population of free purified biological particles

(e) measuring a neutralization titer of the purified exosome population for SARS-CoV-2 virus, in vitro;

(f) administering to the patient a convalescent serum comprising the high titer neutralizing biological particles.

According to some embodiments, the method comprises an initial ultrafiltration step, an initial ultracentrifugation step or both to provide pooled a heterogeneous population of biological particles.

According to some embodiments, the convalescent serum sample is derived from a normal healthy control subject (meaning a subject having no symptoms or other evidence of a severe viral infection, e.g., a control subject not infected with SARS-CoV-2), a subject infected with SARS-CoV-2 (as confirmed by, e.g., PCR) who had developed mild symptoms and recovered, or a subject infected with SARS-CoV-2 (as confirmed by, e.g., PCR) who had developed severe symptoms and recovered. According to some embodiments, the convalescent serum sample has a high titer of antibodies against the Receptor Binding Protein (RBD) of SARS-CoV-2 spike protein. According to some embodiments, the titer of antibodies against the Receptor Binding Protein (RBD) of SARS-CoV-2 spike protein is detectable at least at 1:10,000 dilution of the convalescent serum. According to some embodiments, the convalescent serum sample has a low titer of antibodies against the Receptor Binding Protein (RBD) of SARS-CoV-2 spike protein. According to some embodiments, sera comprising low titer of antibodies against the Receptor Binding Protein (RBD) of SARS-CoV-2 spike protein ranges from less than 1:10,000 dilution to below the limit of quantification, inclusive, in the convalescent serum.

According to some embodiments, the biological sample comprises a body fluid. Examples of body fluid include, without limitation, blood, urine, semen (seminal fluid), vaginal secretions, cerebrospinal fluid (CSF), synovial fluid, pleural fluid (pleural lavage), pericardial fluid, peritoneal fluid, amniotic fluid, saliva, nasal fluid, otic fluid, gastric fluid, and breast milk, feces, sweat, tears, nipple aspirates, or other secreted biological fluids. According to some embodiments, the body fluid is a circulating/secreted body fluid, e.g., blood (whole blood, serum, or plasma), CSF, or lymph. According to some embodiments, the biological sample comprises a body tissue (e.g., diseased tissue).

According to some embodiments, the biological fluid from which biological particles comprising a specific biomarker(s) are selected can be reused, e.g., for subsequent rounds to select other biomarkers, to select small noncoding RNAs comprising miRNAs, to isolate RNAs, DNAs, protein, lipids or a combination thereof etc., without negatively affecting the biological fluid.

According to some embodiments, the binding agent includes a nucleic acid (e.g., DNA, RNA, or a combination thereof), a polypeptide (e.g., an antibody, an antibody fragment, an aptamer polypeptide, or combination thereof) or a biologically degradable polymer.

According to some embodiments, the binding agent is immobilized directly on a solid support. According to some embodiments, the binding agent is immobilized indirectly on a solid support. According to some such embodiments, the binding agent is coupled to a nucleic acid, which is immobilized on a solid support. According to some embodiments, the nucleic acid is immobilized on a solid support by click chemistry. According to some embodiments, the binding agent is an antibody specific for a biological particle antigen. According to some embodiments, the biological particle antigen comprises one or more of CD9, CD63, CD81, CD37, CD82, Alix, ACE-2, Tim4, PLAP and/or other cell surface markers inherited by the biological particles. According to some embodiments, the method includes additional cell-specific antibodies for biological particle sub-population enrichment to detect low abundance biomarkers. According to some embodiments, the method includes larger biofluid material inputs than tested herein. According to some embodiments, the method includes a pre-amplification step to improve the qPCR detection threshold. According to some embodiments, the method includes additional cell-specific antibodies for biological particle sub-population enrichment to detect low abundance biomarkers, larger biofluid material inputs, and a pre-amplification step to improve the qPCR detection threshold.

According to some embodiments, the antibody specific for the biological particle antigen is activated with a dibenzocyclo-octyl (DBCO)-ester, dibenzocyclooctyne (DBCO) molecule, 2-IT (2-iminothiolane), MBS (3-maleimidobenzoic acid N-hydroxysuccinimide ester), SPDP (N-succinimidyl 3-(2-pyridyldithio) propionate), SATA (N-succinimidyl S-acetylthioacetate), SMCC (succinimidyl 4-(N-maleimidomethyl) cyclohexane-1-carboxylate), Sulfo-SMCC, or derivatives thereof. According to some embodiments, the antibody specific for the biological particle antigen is activated with a dibenzocyclo-octyl (DBCO)-ester. According to some embodiments, the DBCO-modified antibody is coupled to a DNA linker by click chemistry. According to some embodiments, the antibody-DNA linker conjugates are bound to streptavidin coated well plates. According to some embodiments, the well plates are pretreated with RNAse A.

According to some embodiments, the isolating/separating step comprises washing thereby separating unbound materials from the biological particles bound to the binding agent.

According to some embodiments, the purified population of biological particles is released from the solid support enzymatically. According to some embodiments, the releasing includes enzymatically cleaving the connection between the one or more binding agent and the solid support. According to some embodiments, the purified population of biological particles is released by enzymatic cleavage of the nucleic acid, which is connected to the binding agent. According to some embodiments, the nucleic acid is DNA. According to some embodiments, the DNA comprises one or more ribonucleic acid nucleotide. According to some embodiments the one or more ribonucleic acid nucleotide is uracil, a restriction site sequence, or a RNA DNA duplex. According to some embodiments the enzyme specifically cleaves the nucleic acid at a ribonucleic acid nucleotide or at a specific restriction site sequence, or a specific RNA or DNA strand when RNA and DNA strands are duplexed. According to some embodiments, the ribonucleic acid nucleotide is a uracil nucleotide. According to some embodiments, the enzyme that cleaves a nucleic acid at a uracil nucleotide is uracil glycosylase. According to some embodiments, the enzyme that cleaves at a specific nucleic acid sequence is a restriction enzyme. According to some embodiments, the enzyme that cleaves a RNA/DNA duplex specifically is a restriction endonuclease or RNase-H.

According to some embodiments, the purified population of biological particles is released by strand displacement. According to some embodiments, a first incumbent strand of the nucleic acid is separated from the second substrate strand of the nucleic acid by a complementary invader strand to the first or second strand of the nucleic acid and an enzyme comprising strand displacement activity. According to some embodiments, the enzyme comprising the strand displacement activity is DNA polymerase, topoisomerase, or helicase. According to some embodiments, a polymerase chain reaction using an oligonucleotide that is complementary to the region of the DNA attached to the antibody is used to separate the annealed DNA strands to allow release of the antibody from the platform without damaging the DNA strand attached to the antibody.

According to some embodiments, the biological particles are eluted from the binding agent. For example, the biological particles are eluted by contacting the bound binding agent-biological particle complex with free ligand specific for the binding agent to form the purified population of biological particles.

According to some embodiments, the purified population of biological particles comprise exosome surface protein biomarkers. According to some embodiments, the biological particle surface protein biomarkers include one or more of a tetraspanin (e.g., CD9, CD63, CD81, CD37, CD82), Alix, ACE-2, Tim4, PLAP and/or other cell surface markers inherited by the biological particles. According to some embodiments the purified population of exosomes is at least 50% pure, at least 55% pure, at least 60% pure, at least 65% pure, at least 70% pure, at least 75% pure, at least 80% pure, at least 85% pure, at least 90% pure, at least 91%, 92%, 93%, 94%, 95%, 96%, 97%, 98% 99% or 100% pure. According to some embodiments, the purified population of biological particles is homogeneous.

According to some embodiments, neutralization is determined in vitro by incubating isolated purified biological particles derived from convalescent serum with a cell line infected with SARS-CoV-2 in vitro. The presence and magnitude of an agent that prevents infectivity of the virus is determined by measuring viral particle production after a suitable time period, e.g., 12 hr, 24 hr, 36 hr and/or 72 hr. According to some embodiments, cells infected with neon green SARSCoV-2 reporter virus can be counted and compared to a negative control (infected cells without purified convalescent e biological particles). According to some embodiments, viral particle production quantifiable by fluorescence is decreased by at least 10%, at least 20%, at least 30%, at least 40%, at least 50%, at least 60%, at least 70%, at least 80%, at least 85%, at least 90%, at least 95%, or at least 100% when compared to the negative control). According to some embodiments, neutralization activity of convalescent EVs comprising ACE2-receptors play a role in neutralizing the SARS-CoV-2 virus. According to some embodiments, the biological particles may be effective to remove viral proteins from infected cells, while harboring them on their surface and thus allow presentation of the antigens to the immune system.

According to some embodiments, the convalescent serum comprising neutralizing purified biological particles may enhance the effectiveness of convalescent plasma therapy by about 1.1-fold, 1.2-fold, 1.3-fold, 1.4-fold, 1.5-fold, 1.6-fold, 1.7-fold, 1.8-fold, 1.9-fold, about 2-fold, about 3-fold, about 4-fold, about 5-fold, about 6-fold, about 7-fold, about 8-fold, about 9-fold, and about 10-fold when compared to treatment with convalescent plasma therapy alone.

According to some embodiments, the patient suffering from the severe SARSCoV-2 infection may be treated with an additional therapy. According to some embodiments, the additional therapy comprises a supportive therapy, such as the intravenous administration of fluids, supplemental oxygen, vapor administered via nebulizer, etc.

According to some embodiments, the additional therapy comprises a pharmaceutical composition comprising an anti-viral agent, an immunomodulatory agent or both. According to some embodiments, the anti-viral agent inhibits viral entry, decreases viral load, or both. According to some embodiments, the anti-viral agent is selected from acyclovir, gancidovir, foscarnet; ribavirin; amantadine, azidodeoxythymidine (zidovudine), nevirapine, a tetrahydroimidazobenzodiazepinone (TIBO) compound; efavirenz; remdecivir, lopinavir/ritonavir, umifenovir, favipiravir, ivermectin, and delavirdine.

According to some embodiments, the immunomodulatory agent is a glucocorticoid, or a recombinant interferon. According to some embodiments, the glucocorticoid is a corticosteroid e.g., prednisone, dexamethasone, azathioprine, mycophenolate, mycophenolate mofetil, or combinations thereof.

According to another aspect, the present disclosure also provides a method of treating a patient with a severe coronavirus infection comprising:

(a) determining neutralization titer of a population of biological particles purified from a subject in vitro by:
(1) obtaining a biological sample from the subject comprising biological particles;
(2) contacting the biological samples comprising extracellular vesicles from the subject with a binding agent to one or more biological particle surface antigens; wherein the binding agent is linked to a nucleic acid, and wherein the nucleic acid is immobilized on a solid support;
(3) isolating the biological particles bound by the binding agent from the biological sample by washing;
(4) releasing the biological particles bound to the binding agent of (3);
(5) eluting the bound biological particles from the binding agent to form a population of free purified biological particles
(6) determining a neutralization titer of the population of purified biological particles in vitro;

(b) selecting the subject to receive convalescent plasma therapy comprising neutralizing purified biological particles comprising ACE2 receptors when the neutralization titer of the population of purified biological particles from the subject is insufficient to neutralize the viral infection in vitro;

(c) preparing a purified population of biological particles from convalescent plasma of a convalescent subject by:
(i) obtaining a convalescent serum comprising a high anti-SARSCoV-2 IgG titer from a convalescent subject;
(ii) contacting the convalescent serum with a binding agent directed to one or more biological particles surface antigens;
(iii) isolating the biological particles bound by the binding agent from the biological sample by washing;
(iv) releasing the biological particles bound to the binding agent of (b);
(v) eluting the bound biological particles from the binding agent to form a population of free purified biological particles;

(d) measuring a neutralization titer of the purified biological particle population of step (v) for SARS-CoV-2 virus, in vitro; and

(e) administering to the subject the neutralizing purified biological particles or the convalescent serum comprising the neutralizing biological particles of step (d).

According to some embodiments, the method comprises an initial ultrafiltration step, an initial ultracentrifugation step or both to provide pooled a heterogeneous population of biological particles.

According to some embodiments, the biological sample comprises a body fluid. Examples of body fluid include, without limitation, blood, urine, semen (seminal fluid), vaginal secretions, cerebrospinal fluid (CSF), synovial fluid, pleural fluid (pleural lavage), pericardial fluid, peritoneal fluid, amniotic fluid, saliva, nasal fluid, otic fluid, gastric fluid, and breast milk, feces, sweat, tears, nipple aspirates, or other secreted biological fluids. According to some embodiments, the body fluid is a circulating/secreted body fluid, e.g., blood (whole blood, serum, or plasma), CSF, or lymph. According to some embodiments, the biological sample comprises a body tissue (e.g., diseased tissue).

According to some embodiments, the biological fluid from which biological particles comprising a specific biomarker(s) are selected can be reused, e.g., for subsequent rounds to select other biomarkers, to select small noncoding RNAs comprising miRNAs, to isolate RNA, DNA, protein, lipids, or a combination thereof, etc., without negatively affecting the biological fluid.

According to some embodiments, the binding agent includes a nucleic acid (e.g., DNA, RNA, or a combination thereof), a polypeptide (e.g., an antibody, an antibody fragment, an aptamer polypeptide, or combination thereof) or a biologically degradable polymer.

According to some embodiments, the binding agent is immobilized directly on a solid support. According to some embodiments, the binding agent is immobilized indirectly on a solid support. According to some such embodiments, the binding agent is coupled to a nucleic acid, which is immobilized on a solid support. According to some embodiments, the nucleic acid is immobilized on a solid support by click chemistry. According to some embodiments, the binding agent is an antibody specific for a biological particle antigen. According to some embodiments, the biological particle antigen is one or more of CD9, CD63, CD81, CD37, CD82, Alix, ACE-2, Tim4 PLAP and/or other cell surface markers inherited by the biological particles.

According to some embodiments, the antibody specific for the biological particle antigen is activated with a dibenzocyclo-octyl (DBCO)-ester, dibenzocyclooctyne (DBCO) molecule, 2-IT (2-iminothiolane), MBS (3-maleimidobenzoic acid N-hydroxysuccinimide ester), SPDP (N-succinimidyl 3-(2-pyridyldithio) propionate), SATA (N-succinimidyl S-acetylthioacetate), SMCC (succinimidyl 4-(N-maleimidomethyl) cyclohexane-1-carboxylate), Sulfo-SMCC, or derivatives thereof. According to some embodiments, the antibody specific for the biological particle antigen is activated with a dibenzocyclo-octyl (DBCO)-ester. According to some embodiments, the DBCO-modified antibody is coupled to a DNA linker by click chemistry. According to some embodiments, the antibody-DNA linker conjugates are bound to streptavidin coated well plates. According to some embodiments, the well plates are pretreated with RNAse A.

According to some embodiments, the isolating/separating step comprises washing thereby separating unbound materials in the biological sample from the biological particle bound to the binding agent.

According to some embodiments, the purified population of biological particles is released from the solid support enzymatically. According to some embodiments, the releasing includes enzymatically cleaving the connection between the one or more binding agent and the solid support. According to some embodiments, the purified population of biological particles is released by enzymatic cleavage of the nucleic acid, which is connected to the binding agent. According to some embodiments, the nucleic acid is DNA. According to some embodiments, the DNA comprises one or more ribonucleic acid nucleotide. According to some embodiments the one or more ribonucleic acid nucleotide is uracil, a restriction site sequence, or a RNA DNA duplex. According to some embodiments, the enzyme specifically cleaves the nucleic acid at a ribonucleic acid nucleotide. According to some embodiments, the ribonucleic acid nucleotide is a uracil nucleotide. According to some embodiments, the enzyme that cleaves a nucleic acid at a uracil nucleotide is uracil glycosylase. According to some embodiments, the enzyme that cleaves at a specific nucleic acid sequence is a restriction enzyme. According to some embodiments, the enzyme that cleaves a RNA/DNA duplex specifically is a restriction endonuclease or RNase-H.

According to some embodiments, the purified population of biological particles is released by strand displacement. According to some embodiments, a first incumbent strand of the nucleic acid is separated from a second substrate strand of the nucleic acid by a complementary invading strand to the first or second strand of the nucleic acid and an enzyme comprising strand displacement activity. According to some embodiments, the enzyme comprising the strand displacement activity is DNA polymerase, topoisomerase, or helicase. According to some embodiments, a polymerase chain reaction using an oligonucleotide that is complementary to the region of the DNA attached to the antibody is used to separate the annealed DNA strands to allow release of the antibody from the platform without damaging the DNA strand attached to the antibody.

According to some embodiments, the biological particles are eluted from the binding agent. For example, the biological particles may be eluted by contacting the bound binding agent-biological particle complex with free ligand specific for the binding agent to form the purified population of biological particles.

According to some embodiments, the purified population of biological particles comprise biological particle surface protein biomarkers. According to some embodiments, the biological particle surface protein biomarkers include one or more of a tetraspanin (e.g., CD9, CD63, CD81, CD37, CD82), Alix, ACE-2, Tim4, PLAP and/or other cell surface markers inherited by the biological particles. According to some embodiments the purified population of biological particle is at least 50% pure, at least 55% pure, at least 60% pure, at least 65% pure, at least 70% pure, at least 75% pure, at least 80% pure, at least 85% pure, at least 90% pure, at least 91%, 92%, 93%, 94%, 95%, 96%, 97%, 98% 99% or 100% pure. According to some embodiments, the purified population of biological particles is homogeneous.

According to some embodiments, neutralization is determined in vitro by incubating isolated purified biological particle derived from convalescent serum with cells infected with SARS-CoV-2 in vitro. The presence and magnitude of an agent that prevents infectivity of the virus is determined by measuring viral particle production after a suitable time period, e.g., 12 hr, 24 hr, 36 hr and 72 hr. According to some embodiments, cells infected with neon green SARSCoV-2 reporter virus can be counted and compared to a negative control (infected cells without purified convalescent biological particles). According to some embodiments, to be considered neutralizing, viral particle production quantifiable by fluorescence is decreased by at least 10%, at least 20%, at least 30%, at least 40%, at least 50%, at least 60%, at least 70%, at least 80%, at least 85%, at least 90%, at least 95%, or at least 100% when compared to the negative control. According to some embodiments, neutralization activity of convalescent EVs comprising ACE2-receptors play a role in neutralizing the SARS-CoV-2 virus. According to some embodiments, the biological particles may be effective to remove viral proteins from infected cells, while harboring them on their surface and thus allow presentation of the antigens to the immune system.

According to some embodiments, the convalescent serum sample is derived from a normal healthy control subject (meaning a subject having no symptoms or other evidence of a severe viral infection, e.g., a control subject not infected with SARS-CoV-2), a subject infected with SARS-CoV-2 who had developed mild symptoms and recovered, or a subject infected with SARS-CoV-2 who had developed severe symptoms and recovered.

According to some embodiments, the convalescent serum sample has a high titer of antibodies against the Receptor Binding Protein (RBD) of SARS-CoV-2 spike protein. According to some embodiments, the titer of antibodies against the Receptor Binding Protein (RBD) of SARS-CoV-2 spike protein is detectable at least at a 1:10,000 dilution of the convalescent serum. According to some embodiments, the convalescent serum sample has a low titer of antibodies against the Receptor Binding Protein (RBD) of SARS-CoV-2 spike protein. According to some embodiments, the titer of sera comprising low titer of antibodies against the Receptor Binding Protein (RBD) of SARS-CoV-2 spike protein ranges from less than 1:10,000 dilution to below the limit of quantification, inclusive.

According to another aspect, the present disclosure provides a method of preparing a population of purified cells from a biological sample from a subject comprising:

a) preparing a population of purified cells by:
(1) obtaining a biological sample from the subject comprising cells;
(2) contacting the biological sample from the subject comprising cells with a binding agent directed to one or more cell surface antigens, wherein the binding agent is linked to a nucleic acid by a linker, and wherein the nucleic acid is immobilized on a solid support;

b) isolating the cell bound by the binding agent from the biological sample;

c) releasing the cell bound to the binding agent;

d) eluting the bound cell from the binding agent to form a population of purified cells.

According to some embodiments the population of cells is derived from non-eukaryotic and eukaryotic species (e.g., human, mouse, rat, monkey, etc.), healthy normal tissue (e.g., blood, tissue, etc) cells (including, without limitation, endothelial, epithelial, T cells fibroblasts, adipocytes, neuronal cells and specific tumor cell subpopulations). According to some embodiments, the cells can be derived from normal and diseased tissues from human patients, murine orthotopic/xenograft/PDX organs, blood, or tissue culture and may be purified using EV-CATCHER targeting a unique surface marker, e.g., in situations to identify new clones, to purify unique cells that are too sensitive to be subjected to flow cytometry, or cells that need to be concentrated.

According to some embodiments, the method comprises an initial ultrafiltration or ultracentrifugation step to provide a starting pooled heterogeneous population of cells. According to some embodiments, the biological sample is prepared in vivo or in vitro.

According to some embodiments, the biological sample comprises a body fluid. According to some embodiments, the body fluid comprises whole blood, serum, plasma, cerebrospinal fluid (CSF), lymph, urine, feces, sweat, tears, nipple aspirates, or seminal fluid or a secreted biological fluid. According to some embodiments, the body fluid is a circulating/secreted body fluid. According to some embodiments, the circulating body fluid is whole blood, serum, plasma, cerebrospinal fluid (CSF) or lymph.

According to some embodiments, the method is a high-throughput assay, which allows for the purification of multiple samples at one, i.e., compatible with 24-, 96-, -384 well plate formats or any other multiwall format. According to some embodiments, the biological fluid from which cells comprising a specific biomarker(s) are selected can be reused, e.g., for subsequent rounds to select other biomarkers, to select small noncoding RNAs comprising miRNAs, to isolate RNA, DNA, protein, lipids or a combination thereof, etc., without negatively affecting the biological fluid.

According to some embodiments, the binding agent is an antibody specific for a cell surface antigen. According to some embodiments, the cell surface antigen includes, without limitation, one or more of CD4, CD8, CD9, CD46, CD63, CD81, CD37, CD82, CD138, CD151, ALix, ACE-2, Tim4, PLAP, Adiponectin, FABP4, Caveolin-1, Cytokeratins, EPCAM, E-Cadherin, P63, heterologous cell surface polypeptides and/or other cell surface markers. Exemplary commercially available sources of such antibodies include, without limitation, CD4 (Cell Signaling Technologies #27520), CD8 (Cell Signaling Technologies #81575), CD46 (Abcam #ab271871), CD138 (Abcam #ab242394), CD151 (Abcam #ab27406), Adiponectin (Abcam #ab227051), EPCAM (Cell Signaling Technology #46403). In this way the purification process can be tailored to select specific populations of cells as desired, e.g., infected by a virus, derived from a cancer cell, derived from an organ, and scan their content. According to some embodiments, the method includes additional cell-specific antibodies for the cell sub-population enrichment to detect low abundance biomarkers. According to some embodiments, the method includes larger biofluid material inputs than tested herein. According to some embodiments, the method includes a pre-amplification step to improve the qPCR detection threshold. According to some embodiments, the method includes additional cell-specific antibodies for cell sub-population enrichment to detect low abundance biomarkers, larger biofluid material inputs, and a pre-amplification step to improve the qPCR detection threshold.

According to some embodiments the purified population of cells is at least 50% pure, at least 55% pure, at least 60% pure, at least 65% pure, at least 70% pure, at least 75% pure, at least 80% pure, at least 85% pure, at least 90% pure, at least 91%, 92%, 93%, 94%, 95%, 96%, 97%, 98% 99% or 100% pure. According to some embodiments, the purified population of cells is homogeneous.

According to some embodiments, the heterologous cell surface polypeptides are expressed by chimeric antigen receptor T cells (CAR-T cells).

According to some embodiments, the binding agent that binds to one or more cell surface antigens is an antibody, an antibody binding fragment, or an aptamer. According to some embodiments, the aptamer is a nucleic acid or a polypeptide. According to some embodiments, the nucleic acid comprises DNA, RNA, or a combination thereof. According to some embodiments, the nucleic acid comprises DNA. According to some embodiments, the DNA comprises one or more ribonucleic acid nucleotide. According to some embodiments, the one or more ribonucleic acid nucleotide is uracil. According to some embodiments, the DNA comprises a restriction enzyme recognition site. According to some embodiments, the nucleic acid is a DNA/RNA duplex which can be degraded by an endonuclease or a RNAse (RNase-H). According to some embodiments, the nucleic acid comprises non-natural nucleotides.

According to some embodiments, the nucleic acid further comprises a binding moiety on a first end of the nucleic acid and a binding moiety on a second end of the nucleic acid, and wherein the binding moiety on the first end of the nucleic acid and the binding moiety on the second end of the nucleic acid are different. According to some embodiments, the binding moiety on the first end of the nucleic acid is an avidin, streptavidin or carboxyl binding moiety. According to some embodiments, the binding moiety is biotin. According to some embodiments, the binding moiety on the second end of the nucleic acid is an amine moiety. According to some embodiments, the amine moiety is azide.

According to some embodiments, the binding agent to one or more cell surface antigens comprises a dibenzocyclooctyne (DBCO) molecule, 2-IT (2-iminothiolane), MBS (3-maleimidobenzoic acid N-hydroxysuccinimide ester), SPDP (N-succinimidyl 3-(2-pyridyldithio) propionate), SATA (N-succinimidyl S-acetylthioacetate), SMCC (succinimidyl 4-(N-maleimidomethyl) cyclohexane-1-carboxylate), Sulfo-SMCC, or derivatives thereof. According to some embodiments, the DBCO-modified antibody is coupled to a DNA linker by click chemistry. According to some embodiments, the antibody-DNA linker conjugates are bound to streptavidin coated well plates. According to some embodiments, the well plates are pretreated with RNAse A.

According to some embodiments, the isolating/separating step comprises washing, thereby separating unbound materials in the biological sample from the cells bound to the binding agent. According to some embodiments, the cells bound to the binding agent are released from the solid support enzymatically. According to some embodiments, the releasing includes enzymatically cleaving the connection between the one or more binding agent and the solid support. According to some embodiments, the purified population of cells is released by enzymatic cleavage of the nucleic acid, which is connected to the binding agent. According to some embodiments, the nucleic acid is DNA. According to some embodiments, the DNA comprises one or more ribonucleic acid nucleotide. According to some embodiments the one or more ribonucleic acid nucleotide is uracil, a restriction site sequence, or a RNA DNA duplex. According to some embodiments the enzyme specifically cleaves the nucleic acid at a ribonucleic acid nucleotide or at a specific restriction site sequence, or a specific RNA or DNA strand when RNA and DNA strands are duplexed. According to some embodiments, the ribonucleic acid nucleotide is a uracil nucleotide. According to some embodiments, the enzyme that cleaves the nucleic acid at a uracil nucleotide is uracil glycosylase. According to some embodiments, the enzyme that cleaves a specific nucleic acid sequence is a restriction enzyme. According to some embodiments, the enzyme that cleaves a RNA/DNA duplex specifically is a restriction endonuclease or RNase-H.

According to some embodiments, the purified population of cells is released by strand displacement. According to some embodiments, a first incumbent strand of the nucleic acid is separated from the second substrate strand of the nucleic acid by a complementary invader strand to the first or second strand of the nucleic acid and an enzyme comprising strand displacement activity. According to some embodiments, the enzyme comprising strand displacement activity is DNA polymerase, topoisomerase, or helicase. According to some embodiments, a polymerase chain reaction using an oligonucleotide that is complementary to the region of the DNA attached to the antibody is used to separate the annealed DNA strands to allow release of the antibody from the platform without damaging the DNA strand attached to the antibody.

According to some embodiments, the binding agent is immobilized directly on a solid support. According to some embodiments, the binding agent is immobilized indirectly on a solid support. According to some such embodiments, the binding agent is coupled to a nucleic acid, which is immobilized on a solid support. According to some embodiments, the nucleic acid is immobilized on a solid support by click chemistry. According to some embodiments, the solid support is a well plate, polymer, or a surface.

According to some embodiments, releasing the isolated cell comprises:

(i) enzymatically cleaving the nucleic acid; or

(ii) displacing a first strand of the nucleic acids connected to the antibody from the second strand of the nucleic acids connected to the support by strand displacement with a complementary nucleic acid to the first or second strand of the nucleic acid and an enzyme having strand displacement activity to release the antibody from the support; or

(iii) separating the annealed DNA strands to allow release of the antibody from the platform without damaging the DNA strand attached to the antibody by a polymerase chain reaction using an oligonucleotide complementary to the region of the DNA attached to the antibody.

According to some embodiments, the enzymatic cleaving is with uracil glycosylase. According to some embodiments, the enzymatic cleaving is with a restriction enzyme. According to some embodiments, the enzymatic cleaving is with an endonuclease or a RNAse.

According to some embodiments, the enzyme having strand displacement activity is DNA polymerase, topoisomerase, or helicase.

According to some embodiments, the population of purified cells comprises non-eukaryotic cells, eukaryotic cells, or a combination thereof. According to some embodiments, the eukaryotic cells are human, mouse, rat, dog, non-human primate, or feline. According to some embodiments, the population of purified cells are derived from a healthy subject or a subject suffering from a disease. According to some embodiments, the disease comprise a cancer, viral infection, abnormal placentation, inflammation and other pathologies. According to some embodiments, the cells are endothelial cells, epithelial cells, T-cells, fibroblasts, adipocytes, neuronal cells, tumor cells, blood cells, or cardiac cells.

According to some embodiments, the viral infection is a severe coronavirus infection. According to some embodiments, the severe coronavirus infection is due to SARS-CoV-1, MERS, or SARS-CoV-2. According to some embodiments, the severe coronavirus infection is due to SARS-CoV-2.

According to some embodiments, the population of cells is derived from (a) non-eukaryotic and eukaryotic species; or (b) healthy normal tissue or diseased tissue; or (c) a murine orthotopic/xenograft/PDX organ; or (d) blood; or (e) a tissue culture.

### Formulations

The term “pharmaceutical composition” is used herein to refer to a composition that is employed to prevent, reduce in intensity, cure or otherwise treat a target condition or disease. Generally a pharmaceutical composition comprises an active agent plus a pharmaceutically acceptable carrier.

The term “carrier” as used herein describes a material that does not cause significant irritation to an organism and does not abrogate the biological activity and properties of the active compound of the composition of the described invention. Carriers must be of sufficiently high purity and of sufficiently low toxicity to render them suitable for administration to the mammal being treated. The carrier can be inert, or it can possess pharmaceutical benefits, cosmetic benefits or both. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components. The carrier can be liquid or solid and is selected, with the planned manner of administration in mind, to provide for the desired bulk, consistency, etc., when combined with an active agent and other components of a given composition.

The phrase “pharmaceutically acceptable carrier” is art recognized. It is used to mean any substantially non-toxic carrier conventionally useable for administration of pharmaceuticals in which the isolated EVs of the present invention will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition. Exemplary carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in “Remington's Pharmaceutical Sciences” by E. W. Martin, which is incorporated herein by reference in its entirety. According to some embodiments, the pharmaceutically acceptable carrier is sterile and pyrogen-free water. According to some embodiments, the pharmaceutically acceptable carrier is Ringer's Lactate, sometimes known as lactated Ringer's solution.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, .alpha.-tocopherol, and the like; and metal chelating agents, such as citric acid, ethylenediamine tetraacetic acid (EDTA), sorbitol, tartaric acid, phosphoric acid, and the like.

Specific modes of administration will depend on the indication. The selection of the specific route of administration and the dose regimen is to be adjusted or titrated by the clinician according to methods known to the clinician in order to obtain the optimal clinical response. The amount of active agent to be administered is that amount sufficient to provide the intended benefit of treatment. The dosage to be administered will depend on the characteristics of the subject being treated, e.g., the particular mammal or human treated, age, weight, health, types of concurrent treatment, if any, and frequency of treatments, and can be easily determined by one of skill in the art (e.g., by the clinician).

The term “parenteral” and its other grammatical forms as used herein refers to administration of a substance occurring in the body other than by the mouth or alimentary canal. For example, the term “parenteral” as used herein refers to introduction into the body by way of an injection (i.e., administration by injection), including, for example, subcutaneously (i.e., an injection beneath the skin), intramuscularly (i.e., an injection into a muscle); intravenously (i.e., an injection into a vein), intrathecally (i.e., an injection into the space around the spinal cord or under the arachnoid membrane of the brain), or infusion techniques.

According to some embodiments, a composition according to the present disclosure is administered multiple times, or as needed in the judgment of the treating physician. According to one such embodiment, the composition is administered at a first infusion date, and optionally at a second infusion date, a third infusion date, a fourth infusion date, a fifth infusion date, a sixth infusion date, a seventh infusion date, an eighth infusion date, a ninth infusion date, a tenth infusion date, and so on.

If infused through a catheter, the therapeutic efficacy of the biological particles of the present disclosure depends on the pharmaceutical composition maintaining their potency as they pass through a catheter.

According to some embodiments, the methods of the present disclosure comprise aerosolizing the pharmaceutical composition in a form selected from a dry powder, a suspension or a solution. According to some embodiments, the composition is a solution. According to some embodiments, the composition is a suspension. According to some embodiments, the composition is a dry powder. For administration by inhalation, the compositions for use according to the present disclosure can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch. According to some embodiments, the inhalation delivery device is a nebulizer, a metered-dose inhaler, or a dry powder inhaler (DPI). According to some embodiments, the respirable particles range in size from about 1 to 10 microns, inclusive. According to some embodiments, particles for nasal administration (insufflation), range in size from 10-500 μM, inclusive.

According to some embodiments, the pharmaceutical compositions of the present disclosure comprise one or more therapeutic agents other than the neutralizing purified biological particles as described. For example, according to some embodiments, the additional therapy may comprise a pharmaceutical composition comprising an anti-viral agent, an immunomodulatory agent or both. According to some embodiments, the anti-viral agent inhibits viral entry, decreases viral load, or both. According to some embodiments, the anti-viral agent is selected from acyclovir, gancidovir, foscarnet; ribavirin; amantadine, azidodeoxythymidine/zidovudine), nevirapine, a tetrahydroimidazobenzodiazepinone (TIBO) compound; efavirenz; remdecivir, lopinavir/ritonavir, umifenovir, favipiravir, ivermectin, and delavirdine. According to some embodiments, the immunomodulatory agent is a glucocorticoid, or a recombinant interferon. According to some embodiments, the glucocorticoid is a corticosteroid selected from prednisone, dexamethasone, azathioprine, mycophenolate, mycophenolate mofetil, and combinations thereof.

According to the foregoing embodiments, the pharmaceutical composition may be administered once, for a limited period of time or as a maintenance therapy over an extended period of time, for example until the condition is ameliorated or cured. A limited period of time may be for 1 week, 2 weeks, 3 weeks, 4 weeks and up to one year, including any period of time between such values, including endpoints. According to some embodiments, the pharmaceutical composition may be administered for about 1 day, for about 3 days, for about 1 week, for about 10 days, for about 2 weeks, for about 18 days, for about 3 weeks, or for any range between any of these values, including endpoints.

According to the foregoing embodiments, the composition or pharmaceutical composition may be administered once daily, twice daily, three times daily, four times daily or more.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise. The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Materials and Methods

### Experimental and Clinical Specimens

Human MCF-7 and mouse RAWS 264.7 exosomes were purchased from System Biosciences (Cat#: EXOP-100A-1 and EXOP-165A-1 respectively). MCF-7 exosomes for protein titration experiments were obtained by ultracentrifugation. Briefly, 60 ml of tissue culture media (comprised of DMEM (Gibco, #10566016) supplemented with 10% exosome depleted FBS (Gibco, #A2720801) and 1% PenStrep (Gibco, #15140122)), obtained from ˜80% confluent MCF-7 cells, was refreshed 48 h before collection. Cells, dead cells and cell debris were removed by centrifugation at 500×g for 5 min, 2,000 xg for 10 min, followed by 10,000 xg for 30 min, respectively. Ultracentrifugation was performed in 10 ml Ultraclear tubes (Beckman Coulter, #344059) in an Optima XE-90 ultracentrifuge (Beckman Coulter) for 2 h at 100,000 xg at 4° C. Pellets were washed with 1×PBS (Gibco, #10010023) and then subjected to a second round of ultracentrifugation for 2 h at 100,000 xg at 4° C. and final exosome pellets were resuspended in 50 μl 1×PBS. Serum specimens from SARS-CoV-2 infected patients (first sample set) were collected at Hackensack University Medical Center (HUMC) under IRB #Pro2018-1022 by the HMH Network. Serum samples were processed at the Biorepository (BioR) and stored as 500 μl aliquots at −80° C. Upon submission of an internal proposal (Proposal #BioRCOV4) and approval, 13 serum samples from patients who did not require mechanical ventilation and 17 samples from mechanically ventilated patients with moderate to severe ARDS were collected and both whole sera and EV-CATCHER exosome purification were subjected to RNA extraction. Original convalescent serum specimens (second sample set) were collected from recently recovered Covid-19 individuals enrolled in the convalescent plasma study lead by Dr. Michele Donato (IRB #Pro2020-0375/0378). Remnant samples were transferred to the HMH BioR, after being de-identified and were provided to the Center for Discovery and Innovation (CDI) after review and approval by the Covid-19 Research Review Committee (proposal COVID-19 #102). Provided samples were stored as 1,000 μl aliquots at −80° C. Enzyme-linked immunosorbent assays (ELISA), using the Receptor Binding Domain (RBD) of SARS-CoV-2 spike protein as antigen, developed and optimized to identify high and low IgG serum titers were initially performed. Sera samples were then stratified as either high IgG (titer >10,000) or low IgG (titer Below Limit of Quantification (BLQ)). Three sera samples from high IgG and 3 from BLQ IgG convalescent individuals were subjected to CD63 purification using the EV-CATCHER assay and evaluated for neutralization potential of their exosome content. Two samples (one high and one BLQ) were ultracentrifuged for evaluation of whole exosomes.

Extracellular Vesicle Capture by AnTibody of CHoice and Enzymatic Release (EV-CATCHER) Assay

The release protocol described by Lóf et al. (2017) was further optimized and modified as follows; HPLC-purified uracilated oligonucleotides (Integrative DNA Technology) for the 5′-Azide (5′ Az-AAAAACGAUUCGAGAACGUGACUGCCAUGCCAGCUCGUACUAU CGAA (SEQ ID NO: 1)) and 3′-Biotin (5′Bio-CGAUAGUACGAGCUGGCAUGGCAGUCACGUUCUCGAA UCGUUUU (SEQ ID NO: 2)), adapted from Löf et al. (2017) were resuspended in RNase-free water at a concentration of 250 ng/μl. Equimolar amounts (1:1 ratios) of each oligo were annealed (90° C. for 2 mM, 90-42° C. for 40 mM, 42° C. for 120 min) in 1×RNA annealing buffer (60 mM KCl, 6 mM HEPES (pH 7.5), 0.2 mM MgCl₂), prior to separation on a 15% non-denaturing polyacrylamide (PAGE) gel (0.5×TBE (ThermoFisher, #15581044) at 450 volts for 90 min). The double stranded (ds) DNA linker was visualized on a blue light box with SYBR® Gold™ dye (ThermoFisher, #S11494), excised, centrifugally crushed using a gel breaker tube (IST Engineering, #3388-100) and resuspended in 400 mM NaCl and shaken overnight (0/N) on a thermomixer set to 4° C. and 1,100 RPM. The solution was filtered, and the dsDNA linker was purified using the QIAEX® II gel extraction kit (Qiagen, #20021) according to manufacturer instructions. Purified dsDNA linker was evaluated on a NanoDrop 2000 and diluted to 250 ng/μl. Antibodies (1 mg/ml) used for exosome pulls (anti-CD63 (Abcam, #ab59479; RRID:AB_940915), anti-CD81 (Abcam, #ab233692) and anti-CD9 (Abcam, #ab263023)) were activated using 5 μl of freshly prepared 4 mM DBCO-S-S-NHS ester (Sigma Aldrich, #761532) and incubated for 30 mM at room temperature (RT) in the dark, reactions were stopped by adding 2.5 μl of 1M Tris-Cl (pH 8.0) at RT for 5 mM in the dark. DBCO-activated antibodies were desalted onto pre-equilibrated Zeba desalting columns (ThermoFisher, #89882) by incubation for 1 mM and centrifugation at 1,500×g for 2 mM Antibodies were quantified on a Nanodrop 2000 instrument using protein A280 and antibody-dsDNA (Ab-dsDNA) stock solutions were prepared by conjugating 50 μg of activated antibody with 25 μg of purified DNA linker, 0/N at 4° C. on a rotator. Validation of Ab-dsDNA binding was performed by PAGE where the Ab-dsDNA (1 μg) product was run under non-denaturing and non-reducing conditions, followed by Coomassie (Bio-Rad #1610786) staining to visualize the shift in Ab-dsDNA migration. The next day, Ab-dsDNA conjugates were bound to streptavidin coated 96-well plates (Pierce, #15120). Either single anti-CD63 antibody (1 μg) or a combination of anti-CD63, -CD81 and -CD9 (1 μg of each antibody) (linker bound) was added to single wells in 100 μl 1×PBS. Streptavidin coated 96-well plates with Ab-dsDNA conjugates were placed on a plate shaker at 300 RPM at 4° C., for 8 h to allow for binding to the plate. Solutions were carefully removed, and wells were washed three times with cold 1×PBS solution, prior to addition of RNase-A (12.5 μg/ml) treated samples (100 μl). Plates were sealed using microAMP optical adhesive film (Applied Biosystems, #4311971) and placed on a shaker at 300 RPM at 4° C., overnight. Samples were carefully removed, wells were washed 3 times with cold 1×PBS and 100 μl of freshly prepared uracil glycosylase (UNG) enzyme (ThermoFisher, #EN0362) in 1×PBS (1×UNG buffer (200 mM Tris-Cl (pH 8.0), 10 mM EDTA and 100 mM NaCl), with 1 unit of enzyme) was added to each well. Plates were incubated at 37° C. for 2 h on a shaker at 300 RPM for UNG digest of the dsDNA linker, and exosomes were recovered in this solution for further characterization and downstream analyses.

### RNA Extractions

Whole sera, whole plasma, and isolated exosomes were subjected to total RNA extraction using the miRNeasy Serum/Plasma kit (Qiagen, #217184) according to manufacturer's instructions with some modifications to improve total RNA yield. Briefly, QIAzol was added to 100 μl of whole plasma, serum, or exosome solutions, vortexed and incubated at RT for 5 min, after which chloroform was added to each sample. Samples were vortexed again and incubated at RT for 5 min Samples were centrifuged at 12,000×g, at 4° C. for 15 min and the upper aqueous phase of each sample was carefully removed and transferred into new siliconized tubes, to which 100% ethanol was added. Samples were incubated on ice for 40 min prior to column purification. The clear upper phase was then passed twice through supplied RNeasy minElute columns, washed with RPE (washing buffer for washing membrane-bound RNA and removing trace salts comprising ethanol; Qiagen), and ice cold 80% ethanol. Columns were spun to remove residual ethanol and total RNA was eluted with 50 μl of RNase-free water. Samples were then speed-vacuumed to 10 μl for small-RNA sequencing or to 20 μl for quantitative real-time PCR (RT-qPCR). For cDNA small-RNA library preparation and next-generation sequencing, 3 ng of size primers (1.5 ng-19 nt and 1.5 ng-24 nt; [42]) was spiked into each RNA extraction. For RT-qPCR experiments, 1 pg of ath-miR-159a (IDT, rUrUrUGGArUrUGAAGGGAGCrUCrUA (SEQ ID NO: 3)) was added to QIAzol prior to RNA extractions from MCF-7 and clinical specimens (mild vs severe Covid-19 samples) prior to RNA separation and column purification and used as an exogenous technical normalization control.

### Western Blot Analyses

Western blot analyses were conducted to evaluate presence of exosome surface protein biomarkers from purified exosome fractions. Purified exosomes were lysed and denatured in 1× Laemmli buffer (Bio-Rad, #161-0747) containing 355 mM β-mercaptoethanol, and heated at 95° C. for 5 min. Denatured lysates were pulse centrifuged and separated on 4-12% polyacrylamide precast mini-PROTEAN TGX gel (Bio-Rad, #4561086) by sodium dodecyl sulphate polyacrylamide gel electrophoresis (SDS-PAGE). Five μl of Precision Plus Protein™ Dual Xtra Prestained Protein Standard (Bio-Rad, #1610377) was loaded and used for gel orientation and determination of molecular weights of separated proteins. Samples were loaded and gels were run at 100 V and 400 mA for 90 min (Power Pac 300, Bio-Rad) in 1× Tris/Glycine/SDS buffer (Bio-Rad, #1610732). After the SDS-PAGE run, proteins were transferred to 0.2 μm polyvinylidene fluoride (PVDF) membranes (Bio-Rad, #1704156) using a semidry electro-transfer system (TransBlot Turbo™ v1.02, Bio-Rad) for 30 min at 25 V. Membranes were visualized using the stain-free blot protocol provided on a Chemi-Doc™ MP (Bio-Rad) system to evaluate protein transfer and non-specific binding was prevented by blocking membranes in EveryBlot™ blocking buffer (Bio-Rad, #12010020) for 30 min Membranes were incubated at 4° C. overnight with TBS-T (1×TBS, pH 6.8, 0.1% Tween® 20) diluted anti-mouse primary antibodies (1:1000) targeted against Alix (Life Technologies, #MA183977) and CD63 (Abcam, #ab59479) or with TBS-T diluted anti-rabbit primary antibodies (1:1000) targeted against CD81 (Abcam, #ab233692) and CD9 (Abcam, #ab263023). Membranes were washed with TBS-T (3×5 min) before incubation in either anti-mouse or anti-rabbit IgG horseradish peroxidase conjugated secondary antibodies (1:10000) for 1 h, with gentle agitation at RT. Membranes were washed with TBS-T (3×5 min) before proteins were detected using SuperSignal™ West Femto Maximum Sensitivity Substrate (Pierce, #34095) and protein bands were visualized using ImageLab 4.0 software on a Chemi-Doc MP (Bio-Rad) imaging system.

### Transmission Electron Microscopy

Transmission electron microscopy (TEM) of purified exosomes was performed by Charles River Laboratories (Pathology Associates (PAI), Durham N.C.). Briefly, purified exosomes were fixed using 2% Glutaraldehyde in phosphate buffer (Electron Microscopy Services, #6536-05) and stored at 4° C. 300 mesh formvar-coated grids were inverted onto 20 μl of fixed exosome suspensions for approximately 2 min and wicked dry. Grids were then inverted onto 40 μl of 2% aqueous uranyl acetate for approximately 1 min, and wicked dry. Samples were imaged on a JEOL JEM-1400⁺ transmission electron microscope (JEOL Ltd.; Tokyo, Japan) operating at an accelerating voltage of 80 kV. High resolution TIFF images were acquired and saved using an AMT 16 MP digital camera system (Advanced Microscopy Techniques Corp.; Woburn, Mass.).

### Nanoparticle Tracking

Size and particle concentration of purified exosomes were assessed using a Spectradyne nCS1 instrument. Briefly, 2 μl of purified exosomes were loaded onto TS400 microfluidic cartridges (Spectradyne, #TS400), which allows for the analysis of particles between 65-400 nm. Loaded cartridges were primed in the instrument using 0.2 μm filtered buffer (1×PBS containing 1% Tween® 20) and particle acquisition was carried out. Acquired stats files were analyzed for particle concentration and size distribution using Spectradyne Viewer software, where peak-filtered CSD graphs were generated.

Exosomal Small-RNA cDNA Library Preparation

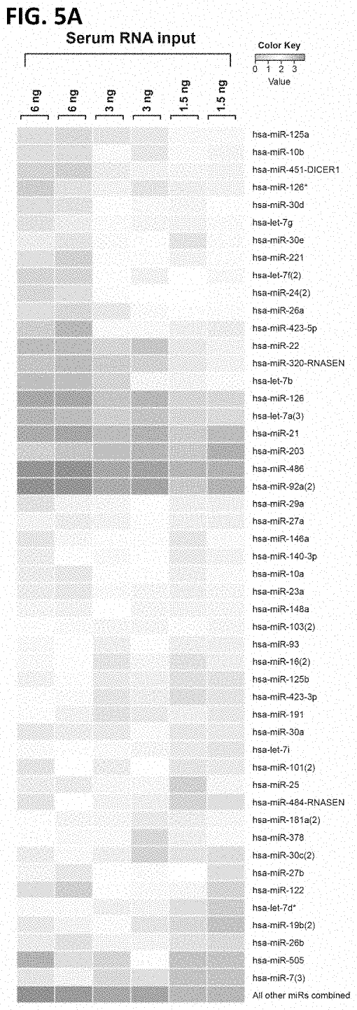

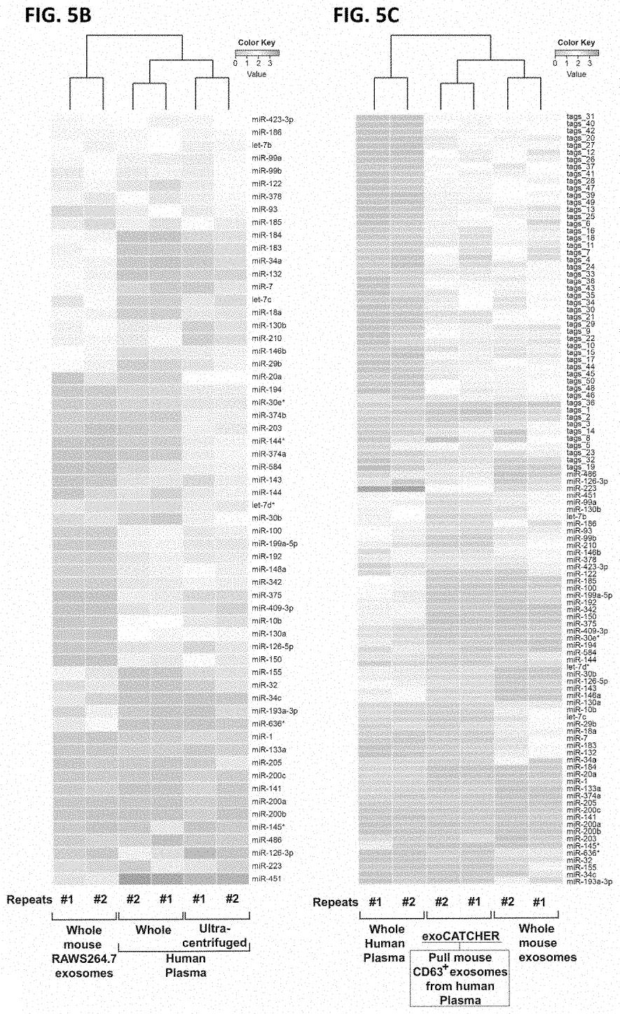

Small-RNA sequencing from exosomes was performed using the cDNA library preparation protocol described by Loudig et al. (2017), with modifications for exosome purifications where RNA amounts are not quantifiable, titration experiments performed in our laboratory indicated that robust small-RNA profiles could be obtained with as little as 5 ng of exosomes (FIG. 5A). For exosomes separately purified from Covid-19 sera, the small-RNA cDNA library preparation was performed using total RNA recovered from exosomes purified from 200 μl of serum (2 wells were prepared for each serum sample (100 μl per well) and pulls were pooled for RNA extraction). Briefly, 15 (Covid-19 libraries) to 18 (optimization experiments) ligations were set up individually by combining 9.5 μl of total RNA, 8.5 μl of the master-mix and 1 μl of 50 μM adenylated barcoded 3′ adapter (Integrated DNA Technologies, custom order). A master-mix was prepared using 0.0052 nM calibrator cocktail (40 μl 10× RNA ligase −2 buffer, 120 μl 50% DMSO, and 10 μl calibrator cocktail from [42]). Reactions were heated at 90° C. for 1 mM, incubated on ice for 2 min, and 1 ( 1/10 diluted) truncated K227Q T4 RNA Ligase 2 (New England Biolabs, #M0351L) was added to each reaction, which were then incubated overnight on ice in a cold room. The next day, ligations were heat inactivated at 90° C. for 1 min, and individually precipitated by addition of 1.2 μl of Glycoblue™ mix (1 μl Glycoblue™ Coprecipitant (15 mg/ml; ThermoFisher, #AM9516) in 26 μl 5 M NaCl (ThermoFisher, #AM9579)) and 63 μl of 100% ethanol was added to each tube. Reactions were combined, precipitated on ice for 1 h and centrifuged for 1 h at 14,000 RPM, at 4° C. The pellet was dried and resuspended in 20 μl nuclease-free water and 20 denaturing PAA gel loading solution and separated on a 15% Urea-PAGE gel. Size marker RNA oligonucleotides (IDT) were used as guide for gel excision. The gel piece was crushed using a gel-breaker tube (IST Engineering, #3388-100) and incubated in 400 mM NaCl overnight at 4° C., at 1,100 RPM on a thermomixer. The next day the solution was filtered and precipitated in 100% ethanol on ice for 1 h. RNA pellet was obtained by centrifugation at 14,000 RPM for 1 h at 4° C. The 5′ adapter was added to the resuspended pellet and T4 RNA Ligase 1 (New England Biolabs, #M0204L) was added for 1 h at 37° C. The ligated product was separated on a 12% Urea-PAGE gel in the presence of 5′ ligated size markers, as guide for size selection. The gel was spun in a gel breaker tube, after which the crushed gel was resuspended in 300 mM NaCl solution with 1 μl of 100 μM 3′ PCR primer, and incubated 0/N on a thermomixer at 1,100 RPM at 4° C. Subsequently, the solution was filtered, precipitated with 100% ethanol, incubated on ice for 1 h and pelleted by centrifugation at 14,000 RPM for 1 h at 4° C. The RNA pellet was resuspended in nuclease free water for reverse transcription (3 μl 5× first strand buffer, 1.5 μl of 0.1M DTT, and 4.2 μl dNTP Mix (2 mM each; ThermoFisher, #R0241)) with 0.75 μl SuperScript® III Reverse Transcriptase (ThermoFisher, #18080-093) and incubated at 50° C. for 30 mM Reverse transcription was deactivated at 95° C. for 1 mM, followed by addition of 95 μl nuclease-free water. A pilot PCR reaction was set up (10 μl 10×PCR buffer, 10 μl dNTP Mix (2 mM each), 10 μl cDNA, 67 μl nuclease-free water, 0.5 μl 3′ PCR primer, 0.5 μl 5′ PCR primer, and 2 μl Titanium® Taq DNA Polymerase (Clontech Laboratories, #639208)). 12 μl aliquots were withdrawn at cycles 10, 12, 14, 16, 18, 20 and 22 for analysis on a 2.5% agarose gel, and identification of the optimal PCR amplification cycle. Six PCR reactions were then set up, run for the optimal number of amplification cycles, a portion (10 μl) was evaluated on a 2.5% agarose gel. The remaining solution was combined, precipitated, digested with PmeI for removal of size markers, and separated on a 2.5% gel. The 100 nt PCR library product was excised, purified with QIAquick Gel Extraction Kit (Qiagen, #28704) and quantified using the Qubit® dsDNA HS Assay Kit (ThermoFisher, #Q32854). cDNA libraries were then sequenced (single-read 50 cycles) on a HiSeq 2500 Sequencing System (Illumina, #SY-401-2501), after which FASTQ files containing raw sequencing data were processed for adapter trimming and small-RNA alignment to the hg-19 genome. Read counts were normalized to total counts and subjected to statistical analyses (see below).

### Quantitative PCR Experiments

Quantitative PCR (qPCR) experiments were performed using TaqMan® microRNA reverse-transcription kits, TaqMan® microRNA master mix PCR kits, and individual TaqMan® microRNA assays following manufacturer's instructions (ThermoFisher). For evaluation of non-specific exosome binding to Dynabeads™ and streptavidin coated 96-well plates, an ath-miR-159a RNA oligo (Integrated DNA Technologies) was used. One picogram of ath-miR-159a was also added during RNA extractions of exosomes purified from Covid-19 infected serum specimens, which was used as a technical normalization control. For optimization experiments we quantified hsa-miR-21 (ThermoFisher, #000397) and hsa-miR-200c (ThermoFisher, #000505) with RNA extracted from MCF-7 exosomes. For validation of differentially expressed miRNAs identified by sequencing of exosomal RNA from Covid-19 serum specimens, TaqMan® miRNA primer assays for hsa-miR-126-3p (#002228), hsa-miR-146a (#002163), hsa-miR-126-5p (#000451), hsa-miR-205 (#000509) were used. For these qPCR reactions, reverse transcriptions were set up using 10% (2 μl out of 20 μl) of RNA extracted from combined CD63/CD81/CD9 exosome purifications from 100 μl of serum. For individual transcript quantifications, ⅓ of the RT reactions (5 μl) was used to set up the three individual qPCR experiments. Quantitative PCR measurements were performed on a Step-One-Plus instrument using manufacturer's recommended streptavidin coated 96-well plates and covers. Quantitative data was transferred to Excel sheet for statistical analyses, as described below. Exemplary miRNA sequences are listed in Table 2 below.

TABLE 2

| Description | Sequence | SEQ ID NO: |
|---|---|---|
| ath-miR-159a | rUrUrUGGArUrUGAAGGGAGCrUCrUA | SEQ ID NO: 3 |
| hsa-miR-21 | UAGCUUAUCAGACUGAUGUUGA | SEQ ID NO: 4 |
| hsa-miR-200c | UAAUACUGCCGGGUAAUGAUGG | SEQ ID NO: 5 |
| hsa-miR-126-3p | UCGUACCGUGAGUAAUAAUGCG | SEQ ID NO: 6 |
| hsa-miR-146a | CCUCUGAAAUUCAGUUCUUCAG | SEQ ID NO: 7 |
| hsa-miR-126-5p | CAUUAUUACUUUUGGUACGCG | SEQ ID NO: 8 |
| hsa-miR-205 | UCCUUCAUUCCACCGGAGUCUG | SEQ ID NO: 9 |
| hsa-miR-125a | UCCCUGAGACCCUUUAACCUGUGA | SEQ ID NO: 10 |
| hsa-miR-106 | UACCCUGUAGAACCGAAUUUGUG | SEQ ID NO: 11 |
| hsa-miR-451-DICER 1 | AAACCGUUACCAUUACUGAGUU | SEQ ID NO: 12 |
| hsa-miR-126* | CAUUAUUACUUUUGGUACGCG | SEQ ID NO: 13 |
| hsa-miR-30d | UGUAAACAUCCCCGACUGGAAG | SEQ ID NO: 14 |
| hsa-let-7g | UGAGGUAGUAGUUUGUACAGUU | SEQ ID NO: 15 |
| hsa-miR-30e | uGUAAACAUCCUUGACUGGAAG | SEQ ID NO: 16 |
| hsa-miR-221 | ACCUGGCAUACAAUGUAGAUUU | SEQ ID NO: 17 |
| hsa-let-7f(2) | UGAGGUAGUAGAUUGUAUAGUU | SEQ ID NO: 18 |
| hsa-miR-24(2) | UGCCUACUGAGCUGAAACACAG | SEQ ID NO: 19 |
| hsa-miR-26a | UUCAAGUAAUCCAGGAUAGGCU | SEQ ID NO: 20 |
| hsa-miR-423-Sp | UGAGGGGCAGAGAGCGAGACUUU | SEQ ID NO: 21 |
| hsa-miR-22 | AGUUCUUCAGUGGCAAGCUUUA | SEQ ID NO: 22 |
| hsa-miR-320-RNASEN | GCCUUCUCUUCCCGGUUCUUCC | SEQ ID NO: 23 |
| hsa-let-7b | UGAGGUAGUAGGUUGUGUGGUU | SEQ ID NO: 24 |
| hsa-miR-126 | CAUUAUUACUUUUGGUACGCG | SEQ ID NO: 25 |
| hsa-let-7 a(3) | UGAGGUAGUAGGUUGUAUAGUU | SEQ ID NO: 26 |
| hsa-miR-203 | AGUGGUUCUUAACAGUUCAACAGUU | SEQ ID NO: 27 |
| hsa-miR-486 | UCCUGUACUGAGCUGCCCCGAG | SEQ ID NO: 28 |
| hsa-miR-92a(2) | GGGUGGGGAUUUGUUGCAUUAC | SEQ ID NO: 29 |
| hsa-miR-29a | ACUGAUUUCUUUUGGUGUUCAG | SEQ ID NO: 30 |
| hsa-miR-27a | AGGGCUUAGCUGCUUGUGAGca | SEQ ID NO: 31 |
| hsa-miR-140-3p | CAGUGGUUUUACCCUAUGGUAG | SEQ ID NO: 32 |
| hsa-miR-10a | UACCCUGUAGAUCCGAAUUUGUG | SEQ ID NO: 33 |
| hsa-miR-23a | GGGGUUCCUGGGGAUGGGAUUU | SEQ ID NO: 34 |
| hsa-miR-148a | AAAGUUCUGAGACACUCCGACU | SEQ ID NO: 35 |
| hsa-miR-103(2) | AGCUUCUUUACAGUGCUGCCUUG | SEQ ID NO: 36 |
| hsa-miR-93 | CAAAGUGCUGUUCGUGCAGGUAG | SEQ ID NO: 37 |
| hsa-miR-16 (2) | UAGCAGCACGUAAAUAUUGGCG | SEQ ID NO: 38 |
| hsa-miR-125b | UCCCUGAGACCCUAACUUGUGA | SEQ ID NO: 39 |
| hsa-miR-423-5p | UGAGGGGCAGAGAGCGAGACUUU | SEQ ID NO: 40 |
| hsa-miR-191 | CAACGGAAUCCCAAAAGCAGCUG | SEQ ID NO: 41 |
| hsa-miR-30a | UGUAAACAUCCUCGACUGGAAG | SEQ ID NO: 42 |
| hsa-let-7i | UGAGGUAGUAGUUUGUGCUGUU | SEQ ID NO: 43 |
| hsa-miR-101(2) | UCGGUUAUCAUGGUACCGAUGC | SEQ ID NO: 44 |
| hsa-miR-25 | AGGCGGAGACUUGGGCAAUUG | SEQ ID NO: 45 |
| hsa-miR-484-RNASEN | UCAGGCUCAGUCCCCUCCCGAU | SEQ ID NO: 46 |
| hsa-miR-181 a(2) | AACAUUCAACGCUGUCGGUGAGU | SEQ ID NO: 47 |
| hsa-miR-378 | CUCCUGACUCCAGGUCCUGUGU | SEQ ID NO: 48 |
| hsa-miR-30c(2) | UGUAAACAUCCUACACUCUCAGC | SEQ ID NO: 49 |
| hsa-miR-27b | AGAGCUUAGCUGAUUGGUGAAC | SEQ ID NO: 50 |
| hsa-miR-122 | UGGAGUGUGACAAUGGUGUUUG | SEQ ID NO: 51 |
| hsa-let-7d* | AGAGGUAGUAGGUUGCAUAGUU | SEQ ID NO: 52 |
| hsa-miR-19b(2) | AGUUUUGCAGGUUUGCAUUUCA | SEQ ID NO: 53 |
| hsa-miR-26b | UUCAAGUAAUUCAGGAUAGGU | SEQ ID NO: 54 |
| hsa-miR-505 | GGGAGCCAGGAAGUAUUGAUGU | SEQ ID NO: 55 |
| hsa-miR-7(3) | UGGAAGACUAGUGAUUUUGUUGUU | SEQ ID NO: 56 |
| miR-423-3p | AGCUCGGUCUGAGGCCCCUCAGU | SEQ ID NO: 57 |
| miR-186 | CAAAGAAUUCUCCUUUUGGGCU | SEQ ID NO: 58 |
| let-7b | UGAGGUAGUAGGUUGUGUGGUU | SEQ ID NO: 59 |
| miR-99a | AACCCGUAGAUCCGAUCUUGUG | SEQ ID NO: 60 |
| miR-99b | CACCCGUAGAACCGACCUUGCG | SEQ ID NO: 61 |
| miR-122 | UGGAGUGUGACAAUGGUGUUUG | SEQ ID NO: 62 |
| miR-378 | CUCCUGACUCCAGGUCCUGUGU | SEQ ID NO: 63 |
| miR-93 | CUCCUGACUCCAGGUCCUGUGU | SEQ ID NO: 64 |
| miR-185 | UGGAGAGAAAGGCAGUUCCUGA | SEQ ID NO: 65 |
| miR-184 | UAUGGAGGUCUCUGUCUGACU | SEQ ID NO: 66 |
| miR-183 | UAUGGCACUGGUAGAAUUCACU | SEQ ID NO: 67 |
| miR-34a | UGGCAGUGUCUUAGCUGGUUGU | SEQ ID NO: 68 |
| miR-132 | AACCGUGGCUUUCGAUUGUUAC | SEQ ID NO: 69 |
| miR-7 | UGGAAGACUAGUGAUUUUGUUGU | SEQ ID NO: 70 |
| let-7c | UGAGGUAGUAGGUUGUAUGGUU | SEQ ID NO: 71 |
| miR-18a | UAAGGUGCAUCUAGUGCAGAUAG | SEQ ID NO: 72 |
| miR-13Ob | ACUCUUUCCCUGUUGCACUAC | SEQ ID NO: 73 |
| miR-210 | AGCCACUGCCCACCGCACACUG | SEQ ID NO: 74 |
| miR-146b | UGAGAACUGAAUUCCAUAGGCUG | SEQ ID NO: 75 |
| miR-29b | GCUGGUUUCAUAUGGUGGUUUAGA | SEQ ID NO: 76 |
| miR-20a | GCUGGUUUCAUAUGGUGGUUUAGA | SEQ ID NO: 77 |
| miR-194 | UAAAGUGCUUAUAGUGCAGGUAG | SEQ ID NO: 78 |
| miR-30e* | CUUUCAGUCGGAUGUUUACAGC | SEQ ID NO: 79 |
| miR-374b | AUAUAAUACAACCUGCUAAGUG | SEQ ID NO: 80 |
| miR-203 | AGUGGUUCUUAACAGUUCAACAGUU | SEQ ID NO: 81 |
| miR-144* | GGAUAUCAUCAUAUACUGUAAG | SEQ ID NO: 82 |
| miR-374a | UUAUAAUACAACCUGAUAAGUG | SEQ ID NO: 83 |
| miR-584 | UUAUGGUUUGCCUGGGACUGAG | SEQ ID NO: 84 |
| miR-143 | GGUGCAGUGCUGCAUCUCUGGU | SEQ ID NO: 85 |
| miR-144 | GGAUAUCAUCAUAUACUGUAAG | SEQ ID NO: 86 |
| let-7d* | CUAUACGACCUGCUGCCUUUCU | SEQ ID NO: 87 |
| miR-30b | UGUAAACAUCCUACACUCAGCU | SEQ ID NO: 88 |
| miR-100 | AACCCGUAGAUCCGAACUUGUG | SEQ ID NO: 89 |
| miR-199a-5p | CCCAGUGUUCAGACUACCUGUUC | SEQ ID NO: 90 |
| miR-192 | CUGACCUAUGAAUUGACAGCC | SEQ ID NO: 91 |
| miR-148a | AAAGUUCUGAGACACUCCGACU | SEQ ID NO: 92 |
| miR-342 | AGGGGUGCUAUCUGUGAUUGA | SEQ ID NO: 93 |
| miR-375 | GCGACGAGCCCCUCGCACAAACC | SEQ ID NO: 94 |
| miR-409-3p | GAAUGUUGCUCGGUGAACCCCU | SEQ ID NO: 95 |
| miR-10b | ACCCUGUAGAACCGAAUUUGUG | SEQ ID NO: 96 |
| miR-130a | GCUCUUUUCACAUUGUGCUACU | SEQ ID NO: 97 |
| miR-126-5p | CAUUAUUACUUUUGGUACGCG | SEQ ID NO: 98 |
| miR-150 | UCUCCCAACCCUUGUACCAGUG | SEQ ID NO: 99 |
| miR-155 | UUAAUGCUAAUCGUGAUAGGGGUU | SEQ ID NO: 100 |
| miR-32 | UAUUGCACAUUACUAAGUUGCA | SEQ ID NO: 101 |
| miR-34c | AGGCAGUGUAGUUAGCUGAUUGC | SEQ ID NO: 102 |
| miR-193a-3p | AACUGGCCUACAAAGUCCCAGU | SEQ ID NO: 103 |
| miR-636* | UGUGCUUGCUCGUCCCGCCCGCA | SEQ ID NO: 104 |
| miR-1 | ACAUACUUCUUUAUAUGCCCAU | SEQ ID NO: 105 |
| miR-133a | GCUGGUAAAAUGGAACCAAAU | SEQ ID NO: 106 |
| miR-205 | UCCUUCAUUCCACCGGAGUCUG | SEQ ID NO: 107 |
| miR-200c | CGUCUUACCCAGCAGUGUUUGG | SEQ ID NO: 108 |
| miR-141 | CAUCUUCCAGUGCAGUGUUGGA | SEQ ID NO: 109 |
| miR-200a | CAUCUUACCGGACAGUGCUGG | SEQ ID NO: 110 |
| miR-200b | CAUCUUACUGGGCAGCAUUGGA | SEQ ID NO: 111 |
| miR-145* | GGAUUCCUGGAAAUACUGUUCU | SEQ ID NO: 112 |
| miR-486 | UCCUGUACUGAGCUGCCCCGAG | SEQ ID NO: 113 |
| miR-126-3p | UCGUACCGUGAGUAAUAAUGCG | SEQ ID NO: 114 |
| miR-223 | CGUGUAUUUGACAAGCUGAGUUG | SEQ ID NO: 115 |
| miR-451 | AAACCGUUACCAUUACUGAGUU | SEQ ID NO: 116 |
| miR486 | UCCUGUACUGAGCUGCCCCGAG | SEQ ID NO: 117 |
| miR-146a | CCUCUGAAAUUCAGUUCUUCAG | SEQ ID NO: 118 |

### BSL-3 Tissue Culture and Neutralization of SARS-CoV-2

### Data Analysis

For sequencing data analysis, raw FASTQ data files obtained from an Illumina HiSeq2500 sequencer were processed using an RNAworld server, including adapter trimming and read alignments and annotation. miRNA counts were exported to spreadsheets for data analysis. Statistical analyses of miRNA counts were performed using dedicated Bioconductor packages in the R platform, as detailed below. Heat maps were generated from transformed counts using the ‘NMF’ package (a heatmap function). Differential expression was assessed using ‘DESeq2’ and ‘edgeR’. Differential expression models included a batch variable (library) to reduce batch biases. Interactions with sex and age were tested [43]. To maximize the discrimination ability of miRNA we computed a score for each sample (‘miRNA score’, [44]), assembled by summing the standardized levels (z-values) of all significantly upregulated miRNA, and the negative of the z-values of all significantly downregulated miRNA. For quantitative PCR (qPCR) we subtracted the threshold cycle (Ct) value of ath-miR-159a (exogenous normalization control) from the Ct value of each individual miRNA and calculated the ΔΔCt comparative method values. qPCR differential expression was assessed via t tests or Mann Whitney U tests of ΔΔCt values. For convalescent exosome neutralization, statistical analysis was performed using Graphpad Prism 8.0 software, where data was assessed using mean±standard error of the mean (SEM). All in vitro experiments consisted of both technical (three separate wells) and biological (three separate experiments) replicates. One-way analysis of variance (ANOVA) with Bonferroni post hoc test was conducted. A p-value <0.05 was considered statistically significant.

### Results

Development of the EV-CATCHER Assay for Purification of Exosomes from Human Biofluids

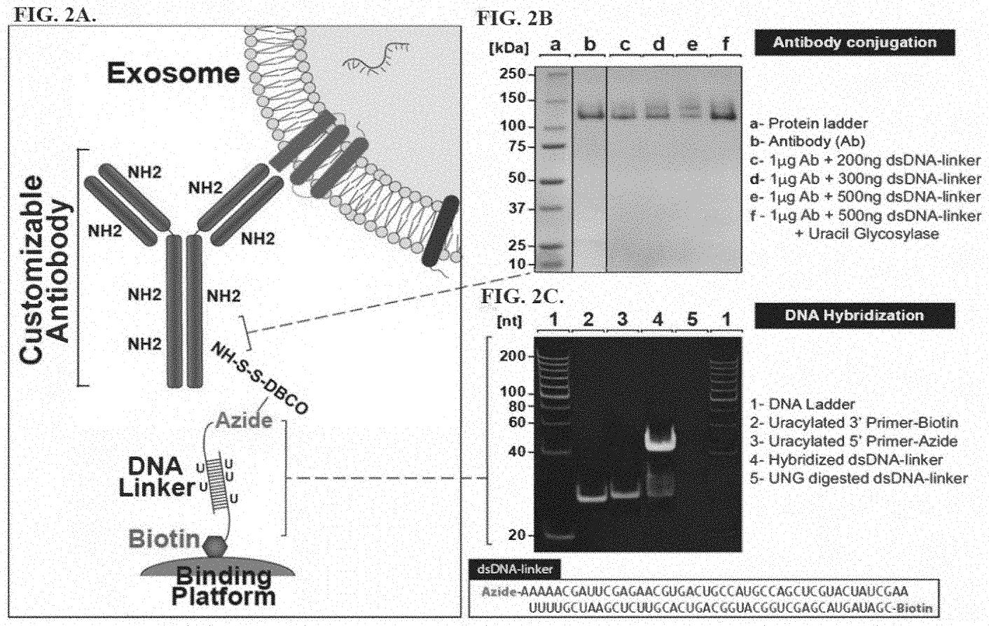

We endeavored to optimize the customizable attachment of selective antibodies to a binding platform for specific purification of exosomes from biological fluids. Thus, using the design described by Löf et al., 2017 (FIG. 2A) we initially established optimal hybridizing conditions to generate the highest amount of the double-stranded DNA linker for subsequent assays (FIG. 2B, lane 4). In order to increase purity of the dsDNA and to prevent representation of single-stranded 5′ azide oligonucleotides, which could bind to the DBCO-activated antibodies and prevent their binding to the platform, we purified the annealed dsDNA products on a non-denaturing acrylamide gel. Using uracil-glycosylase (UNG), we validated the degradable nature of the dsDNA linker through enzymatic digestion (FIG. 2B, lane 5). Next, we determined the optimal ratio between dsDNA-linker and the DBCO activated antibodies, which was evaluated on a 12% PAGE (FIG. 2C, compare lanes c, d and e). The binding of the ds-DNA linker modified the migratory pattern of the antibody, which could be restored upon UNG glycosylase treatment and digestion of the dsDNA linker (FIG. 2C, lanes f). These experiments were repeated with different antibodies and validated our optimized conditions for preparation of ds-DNA linker and antibody duplexes.

### Identification of a Low Non-Specific Nucleic Acid Binding Platform

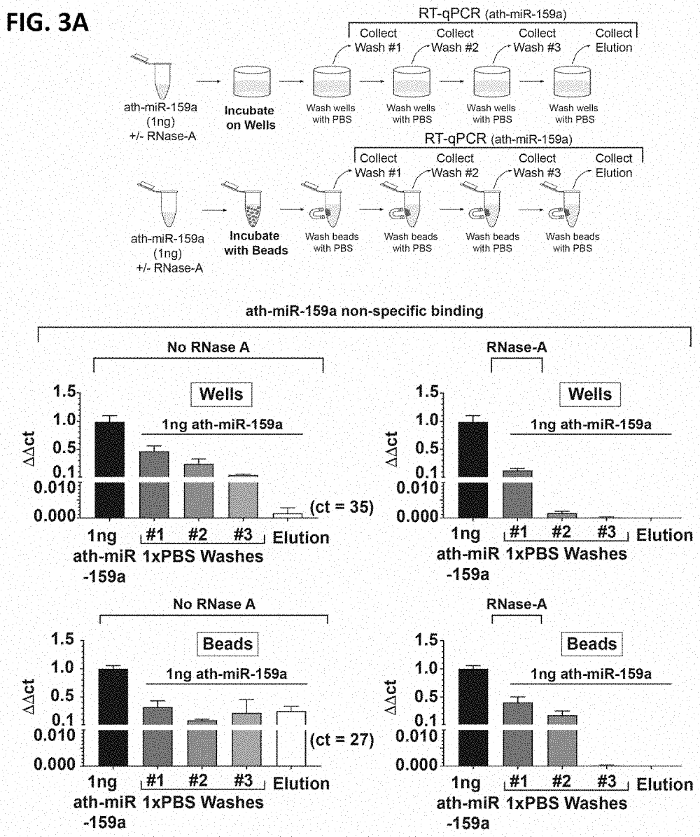

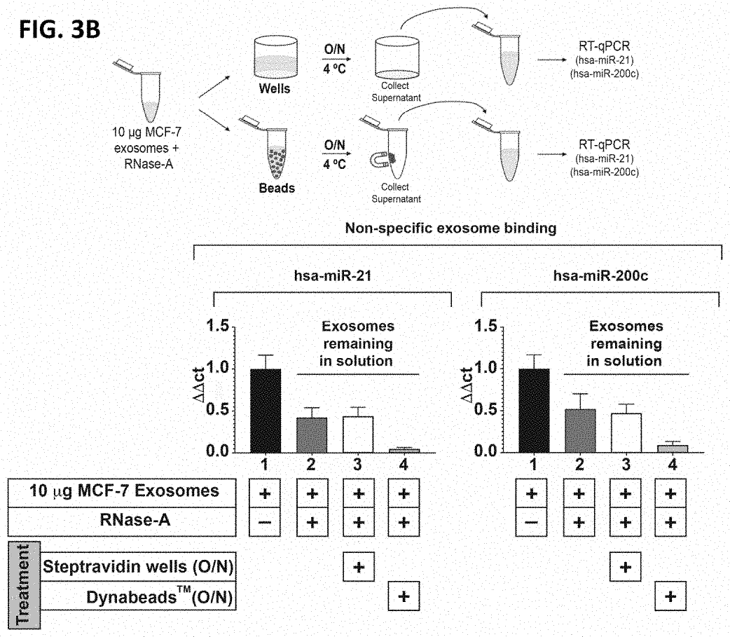

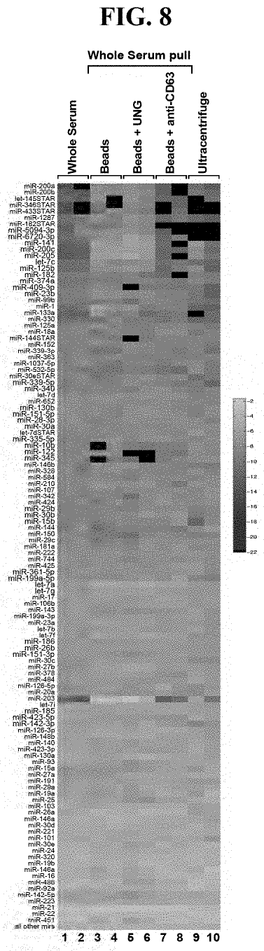

Although magnetic beads are extensively used for the isolation of exosomes prior to the evaluation of their cargos, repeated experiments conducted in our laboratory suggested high levels of non-specific small-RNA binding in the absence of a capture antibody (FIG. 8). Thus, we sought to investigate the non-specific binding capacity of magnetic beads by comparing non-magnetic streptavidin coated 96-well plates (wells; largely used for ELISA assays) to Dynabeads™ (magnetic beads) MyOne™ Streptavidin T1 beads (beads). We chose to use an ath-miR-159a RNA oligonucleotide, an Arabidopsis-thaliana specific miRNA not found as a contaminant in the laboratory (control PCR experiments), as our source of small-RNA for evaluation of the two platforms (beads vs wells). Using qPCR as a sensitive measuring tool, we determined that 1 ng of ath-miR-159a (Ct of 14 cycles; FIGS. 3A and 3B—See black bars in all graphs) was optimal for quantification of non-specific binding to wells and beads. For the evaluation of non-specific ath-159a RNA binding, we collected four fractions including three subsequent PBS washes and one final elution after UNG digest, from wells (FIG. 3A, top left) or beads (FIG. 3A, bottom left). Our data show that the wells retained the least amount of ath-miR-159a RNA, particularly in the final elution (FIG. 3A top and bottom left, light grey bars), with large amounts of ath-miR-159a RNA bound non-specifically and released at all steps, but particularly released during final elution with the beads. We found that treating wells and beads with RNase-A prior to incubation with ath-miR-159a (FIG. 3A, top and bottom right) mostly eliminated non-specific binding of ath-159a RNA and its high representation in the final elution, with beads. This pre-treatment suggests a carry-over of RNase-A activity in the elution, which may not be suitable for all subsequent experiments but appears necessary when using magnetic beads. We then sought to determine if either wells or beads could also bind exosomes non-specifically (FIG. 3B). We performed qPCR evaluations of hsa-miR-21 (Ct=24) and hsa-miR-200c (Ct=27), which we previously detected in exosome RNA extracts from MCF-7 cells. Considering that commercially purified MCF-7 exosomes are obtained by ultracentrifugation, we expected exosome-free hsa-miR-21 and hsa-miR-200c, and thus pre-treated all reactions with RNase-A. Our data indicate that approximately half the miRNA signal remained, suggesting protection from RNase-A activity likely due to exosome bi-lipid layers (FIG. 3B, dark grey bars). The RNase-A treated exosome solutions were incubated in wells or with beads prior to removal, after which the hsa-miR-21 and hsa-miR-200c RNA levels remaining in the solution were evaluated. Our data systematically identified that both miRNAs remained at the same detectable levels after incubation of the exosome solution onto wells, whereas a large proportion of the miRNA signals were lost after incubation and removal of the beads (FIG. 3B, light grey bars). These data suggest that magnetic beads have the capacity to bind both small-RNAs and exosomes non-specifically, which may contribute to non-specific RNA background that we observed when performing small-RNA sequencing of beads incubated in serum (FIG. 8). Therefore, for all subsequent experiments we selected streptavidin coated 96-well plates for immuno-purification of exosomes from all biological fluids evaluated.

Reproducibility and Sensitivity of the EV-CATCHER Assay for Exosome Purification from Biological Fluids

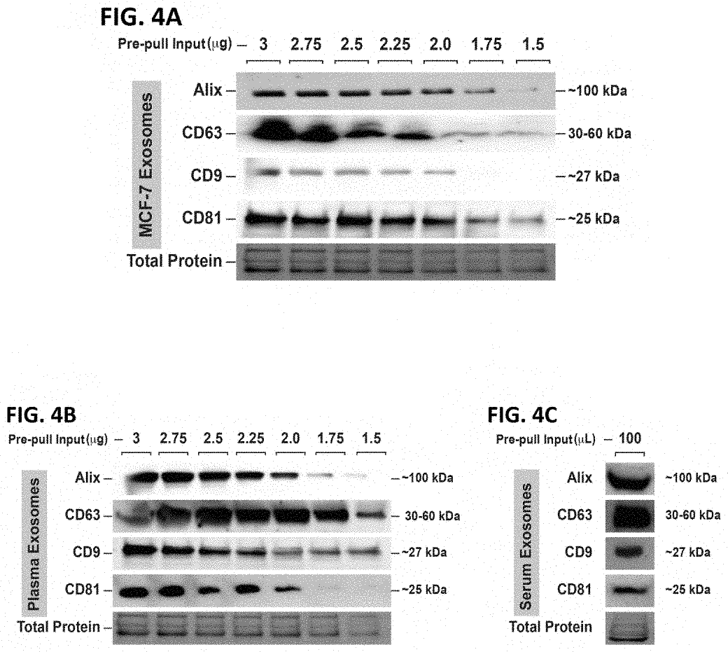

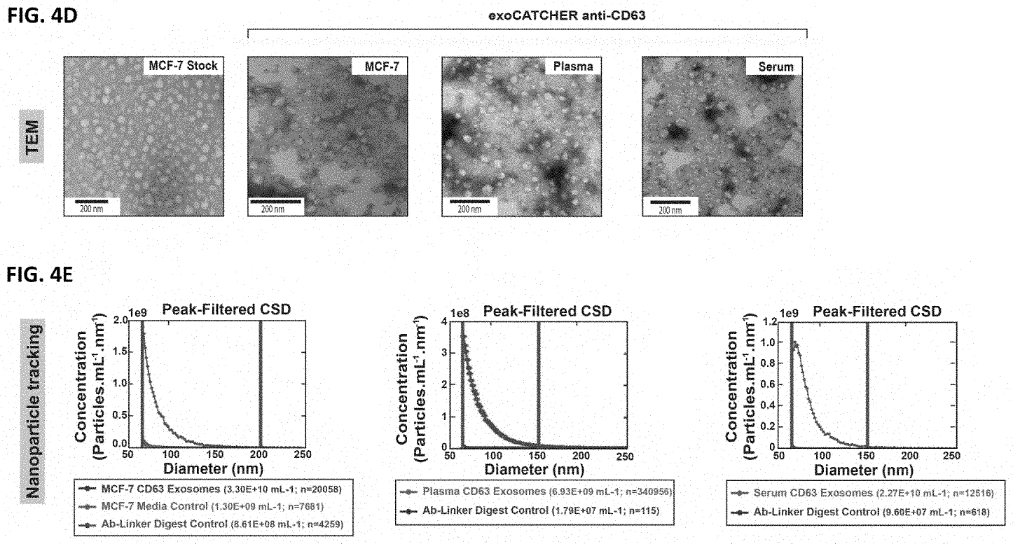

For these proof-of-principle experiments, we used an anti-CD63 antibody on the EV-CATCHER assay to evaluate the purification of exosomes from three different biological sources (tissue culture media of a human cell line (MCF-7), human plasma, and human serum) (FIGS. 4A-4E). We performed these exosome purifications in the presence of decreasing amounts of total protein inputs (quantified by NanoDrop spectrophotometer) and evaluated the reproducibility of the CD63 pulldowns by Western blotting, using anti-Alix, -CD63, -CD9, and -CD81 antibodies for protein expression in exosome elutions (FIGS. 4A-4C). Exosome purification was performed with a single volume of serum (100 μl), due to limited availability. We used transmission electron microscopy (TEM, FIG. 4D) to reveal the presence of intact EVs with size and morphology consistent with previous exosome evaluations (36). Finally, using a Spectradyne nCS1 nanoparticle tracker we evaluated the size distribution of the CD63⁺ purified and released exosomes, which revealed the presence of nanoparticles with sizes ranging between 65-150 nm for exosomes from MCF-7 cell media (3.30×10¹⁰ particles/ml (n=20,058)), plasma (6.93×10⁹ particles/ml (n=340,956), and serum (2.27×10¹⁰ particles/ml (n=12,516)) by comparison to UNG digested Ab-linker from the wells incubated with PBS (FIG. 4E). Collectively these analyses revealed uniform reproducibility of the EV-CATCHER assay for purifying exosomes from different biofluids.

Small-RNA Sequencing Using CD63⁺ Exosomes Purified with EV-CATCHER

Considering that our previous small-RNA sequencing experiments (FIG. 8) using total RNA extracted from exosomes purified with magnetic beads indicated the presence of high small-RNA background, we sought to evaluate the specificity of the EV-CATCHER assay using the same small-RNA cDNA library preparation protocol [42]. As an initial test we validated the applicability of our cDNA library preparation protocol to the detection of circulating small-RNAs using decreasing amounts of total RNA extracted from whole serum (FIG. 5A). Duplicate experiments demonstrated the reproducibility of our sequencing pipeline with small-amounts of circulating small-RNA. Next, we compared small-RNA sequencing data from RNA extracted from ultracentrifuged human plasma (classical purification of whole exosomes) and whole plasma and identified specific miRNA expression differences (FIG. 5B), which further corroborated that the RNA extraction method influences the expression output [45]. As a measure of control, we validated the presence of specific small-RNA expression differences between mouse and human exosomes (FIG. 5B). Finally, we sought to determine if we could specifically select a sub-population of exosomes from a biofluid using EV-CATCHER. For these experiments we chose to spike human plasma with mouse RAWS264.7 exosomes. We customized the EV-CATCHER assay with an anti-mouse CD63 antibody, without cross-reactivity to human CD63, and carried out the purification of mouse exosomes from human plasma. For sensitive evaluation of the purified miRNA cargos, we used our cDNA library preparation protocol and sequenced the small-RNA content of the selected exosomes (FIG. 5C). For these experiments, all libraries were duplicated using fresh (Repeats #1, 3 years old) and older (Repeats #2, 6 years old) 3′ barcoded adapters to accentuate inherent variabilities of the cDNA library preparation protocol. As observed in FIG. 5C, small-RNA expression data from mouse CD63⁺ exosomes captured from human plasma using the EV-CATCHER assay contained mouse-specific small-RNA sequences (Tags), identified solely in mouse RAWS264.7 whole exosomes (FIG. 5C, columns 2 and 3). We also noted that miRNAs differentially detected in whole human plasma (not detectable in mouse RAWS264.7 exosomes) were not detectable within our mouse CD63⁺ purified exosomes. Interestingly, miRNA expression differences were noted between whole RAWS264.7 exosomes (representing a pool of exosomes) and CD63⁺ purified exosomes, with specific miRNAs only detectable in pooled exosomes from mouse RAWS264.7 cells. These next-generation sequencing experiments demonstrate the reproducible detection of circulating small-RNAs and specificity of the EV-CATCHER assay for selected capture of exosomes subpopulations from biofluids.

MiRNA Analysis of Serum Exosomes from Mildly and Severely Ill Covid-19 Patients

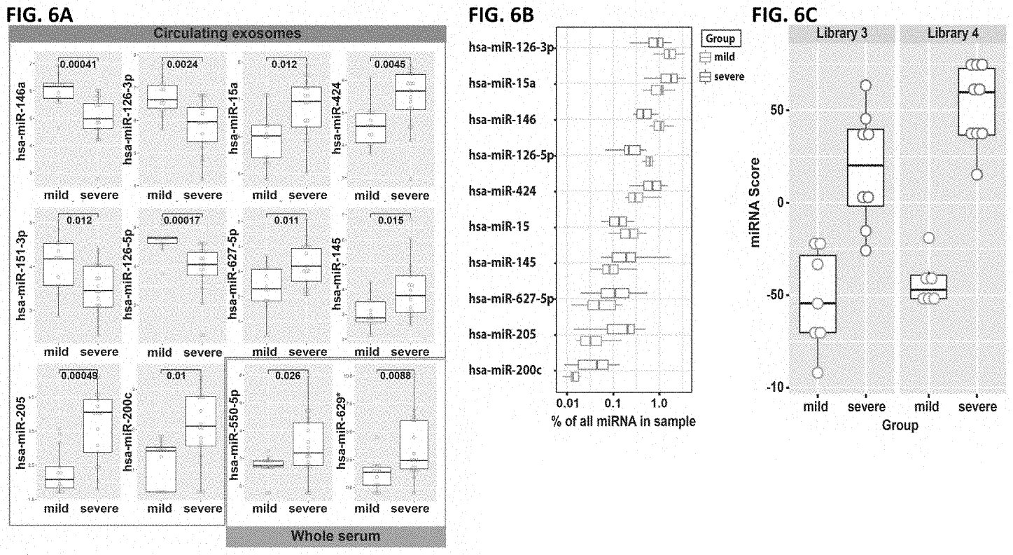

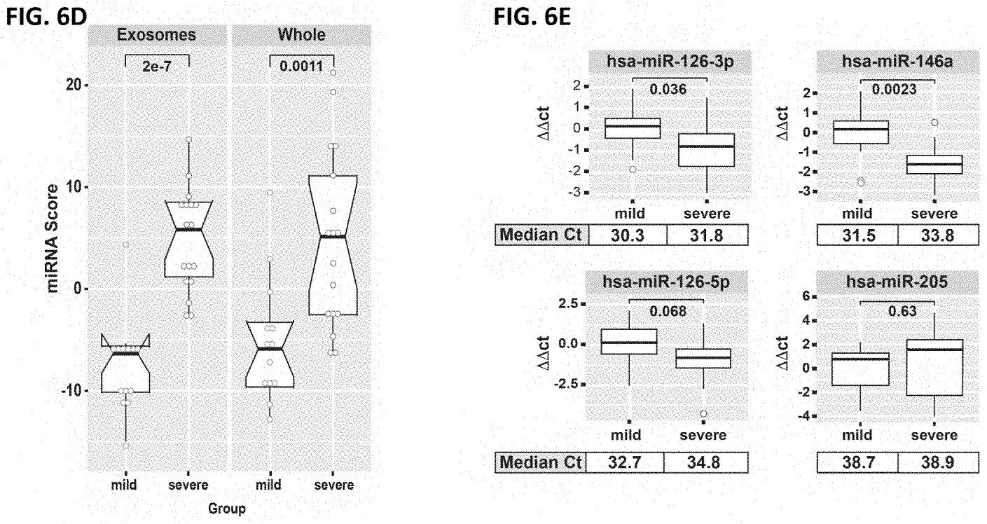

The location of our institute and its parent hospital network within the early U.S. epicenter of the SARS-CoV-2 pandemic, afforded us with the unique opportunity to explore pressing biological questions and further investigate the clinical utility of the EV-CATCHER assay for identification of miRNA biomarkers in serum samples from Covid-19 patients treated within our network. An important question with Covid-19 infection (which has been associated with a wide range of patient outcomes) was to evaluate if circulating miRNA expression changes could be useful for early identification of patients at risk of severe disease. As such we established a first pilot study where we purified circulating exosomes from the serum of mildly and severely affected Covid-19 patients (our SARS-CoV-2 infected serum sample set) and evaluated their miRNA expression content by next-generation sequencing. As proof-of-principle, we chose to use a combination of anti-CD63, -CD81, -CD9 antibodies for unbiased capture of circulating exosomes from serum. To measure the sensitivity of the EV-CATCHER assay we measured miRNA expression differences between mildly and severely ill patients using total RNA extracted from small-EVs (EV-CATCHER) and from whole serum. Mildly ill patients were hospitalized but did not require mechanical ventilation (n=13) while severely ill patients displayed Acute Respiratory Distress Syndrome (ARDS; following Berlin classification standards [46] and required mechanical ventilation (n=17), and both groups had PCR-confirmed infection with the SARS-CoV-2 virus. Clinical data was obtained from these patients (Table 1). Small-RNA cDNA libraries were prepared with 15 individual samples from RNA extracted from whole serum (30 samples; 2 libraries) or from serum exosomes (30 samples; 2 libraries) with each library having even representation of mild and severe RNA samples, to minimize batch effects. Our expression analyses identified 10 differentially expressed miRNAs between mild and severe sample groups using total RNA from serum exosomes (FIG. 6A; hsa-miR-146a (p-val=0.00041), hsa-miR-126-3p (p-val=0.0024), hsa-miR-424 (p-val=0.00454), hsa-miR-151-3p (p-val=0.012), hsa-miR-126-5p (p-val=0.00017), hsa-miR-627-5p (p-val=0.011), hsa-miR-145 (p-val=0.015), hsa-miR-205 (0.00049), and hsa-miR-200c (p-val=0.01)) and only 2 differentially expressed miRNAs between mild and severe sample groups using total RNA from whole serum (FIG. 6A; hsa-miR-550-5p (p-val=0.026), hsa-miR-629* (p-val=0.0088)). As shown in FIG. 6B, the 10 differentially expressed exosomal miRNAs between the two groups appeared in low abundance but reproducibly detectable. We established an integrative miRNA signature using the 10 differentially expressed miRNAs in serum exosomes of mild and severe patients (FIG. 6C). Evaluation of this signature between both exosomal small-RNA libraries confirmed expression differences observed between mild and severe sample groups (FIG. 6C). Interestingly, when we evaluated this miRNA integrative signature for both serum exosomes and whole sera (FIG. 6D), we confirmed the significant difference between mild and severe sample groups in serum purified exosomes (FIG. 6D, p-val=2e-07). Surprisingly from whole sera (p-val=0.011), these 10 miRNAs individually did not display expression differences between the two patient groups. Finally, we utilized gold-standard quantitative PCR approach to evaluate the top 4 differentially expressed miRNAs identified in serum exosomes (FIG. 6E). In order to enable full technical validation of our findings, we activated fresh anti-CD63, -CD81 and -CD9 antibodies, which had been initially used for purification of serum exosomes, and prepared fresh exosome purifications using EV-CATCHER from the same 30 whole sera samples (mild n=13, severe n=17) and extracted total RNA. Using TaqMan® assays we evaluated expression differences of hsa-miR-146a, hsa-miR-126-5p, hsa-miR-126-3p, hsa-miR-205 and successfully validated the top two differentially expressed miRNAs (hsa-miR-146a (pval=0.0023) and hsa-miR-126-3p (0.036)) (FIG. 6E, top two box plots). To evaluate a possible reason for the lack of validation for the two other miRNAs, we displayed the mean of the Ct values for qPCR of the 30 specimens for all 4 miRNAs. We observed that only miRNAs with the lowest Ct values (most expressed) could be validated (FIG. 6E top box plots, see Ct values), which may suggest that these miRNA transcripts were below qPCR detection thresholds. Altogether, our sequencing analyses demonstrate that serum exosomes contain low-abundance specific miRNAs that can be captured when using the EV-CATCHER assay and validated by qPCR. Our analyses also suggest a differential miRNA expression between serum exosomes from mildly and severely ill Covid-19 patients.

TABLE 1

| Mild | Age | Gender | Assisted Ventilation | Duration of Hospitalization (days) | *Comorbidities |
|---|---|---|---|---|---|
| HUMC20 | 27 | F | No | 11 | Ash, DM, Endo, PCOS |
| HUMC23 | 41 | M | No | 12 | No PMHx |
| HUMC25 | 54 | F | No | 2 | DM, HL, HTN, UF |
| HUMC28 | 73 | F | No | 10 | D, HTN, TD |
| HUMC29 | 57 | M | No | 10 | No PMHx |
| HUMC30 | 87 | M | No | 10 | C, BR, DVT, CVA, HL, HTN, Ob |
| HUMC34 | 46 | M | No | 12 | RFH, KD, DN, DM, ED, HM, HC, HTN |
| HUMC39 | 54 | M | No | 7 | HTN |
| HUMC41 | 48 | F | No | 8 | AN, CH, D, SOB, OSA, PN |
| HUMC69 | 60 | M | No | 2 | D, DM, HTN, LTR |
| HUMC127 | 55 | F | No | 7 | Ash |
| HUMC130 | 65 | M | No | 11 | DM, GERD, HTN, OSA |
| HUMC43 | 54 | F | No | 10 | — |
| — | — | — | — | — | — |
| — | — | — | — | — | — |
| — | — | — | — | — | — |
| — | — | — | — | — | |

| Severe | Age | Gender | Assisted Ventilation | Duration of Hospitalization (days) | *Comorbidities |
|---|---|---|---|---|---|
| HUMC42 | 53 | M | Yes | 17 | HD, C, DM, GERD, HL, HTN, |
| | | | | | Ob, OSA, SOB |
| HUMC44 | 63 | M | Yes | 16 | Ash, DM, ET, HT, HTN, OSA |
| HUMC75 | 75 | M | Yes | 10 | No PMHx |
| HUMC76 | 58 | M | Yes | 21 | No PMHx |
| HUMC114 | 54 | M | Yes | 29 | No PMHx |
| HUMC125 | 57 | M | Yes | 22 | C, HL, HTN |
| HUMC309 | 85 | F | Yes | 29 | HD, DM, HC, GERD |
| HUMC314 | 76 | M | Yes | 8 | CAD, HL, HTN, MFL, RLS |
| HUMC315 | 75 | M | Yes | 19 | HD, CHF, FR, HTN, VSU |
| HUMC324 | 62 | M | Yes | 13 | ACS, HTN |
| HUMC326 | 75 | M | Yes | 20 | DM, HL, HTN |
| HUMC327 | 69 | M | Yes | 14 | GERD, HC |
| HUMC328 | 78 | M | Yes | 14 | HTN |
| HUMC342 | 78 | M | Yes | 19 | HTN |
| HUMC37 | 74 | M | Yes | 18 | HTN |
| HUMC123 | 81 | F | Yes | 13 | No PMHx |
| HUMC24 | 54 | M | Yes | 10 | No PMHx |

*Comorbidities are defined by the following lettering: Ash for Asthma, DM for diabetes mellitus, Endo for endometriosis, PCOS for poly cystic ovary syndrome, PMHx for past medical history, HL for hyperlipidemia, HTN for hypertension, TD for thyroid disease, C for cancer, BR for bronchitis, DVT for deep vain thrombosis, CVA for cerebrovascular accident, Ob for obesity, RFH for respiratory failure with hypoxia, KD for kidney disease, DN for diabetic nephropathy, ED for erectile dysfunction, HM for hear murmur, HC for hypercholesterolemia, AN for anxiety, CH for chronic headaches, D for depression, SOB for shortness of breath, OSA for obstructive sleep apnea, PN for pneumonia, LTR for liver transplant recepient, GERD for gastroesophogeal reflux disease, HD for heart disease, ET for ear tumor, HT for high triglycerides, CAD for chronic artery disease, MFL for muscle function loss, RSL for restless leg syndrome, CHF for congestive heart failure, FR for fractures, VSU for venous stasis ulcers, ACS for abnormal cardiovascular stress.

Exosomes Purified with EV-CATCHER Maintain Functional Properties for In Vitro Analyses

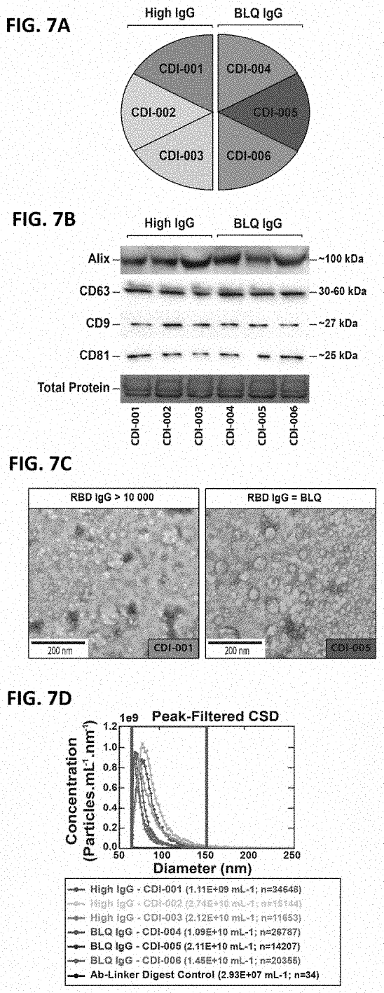

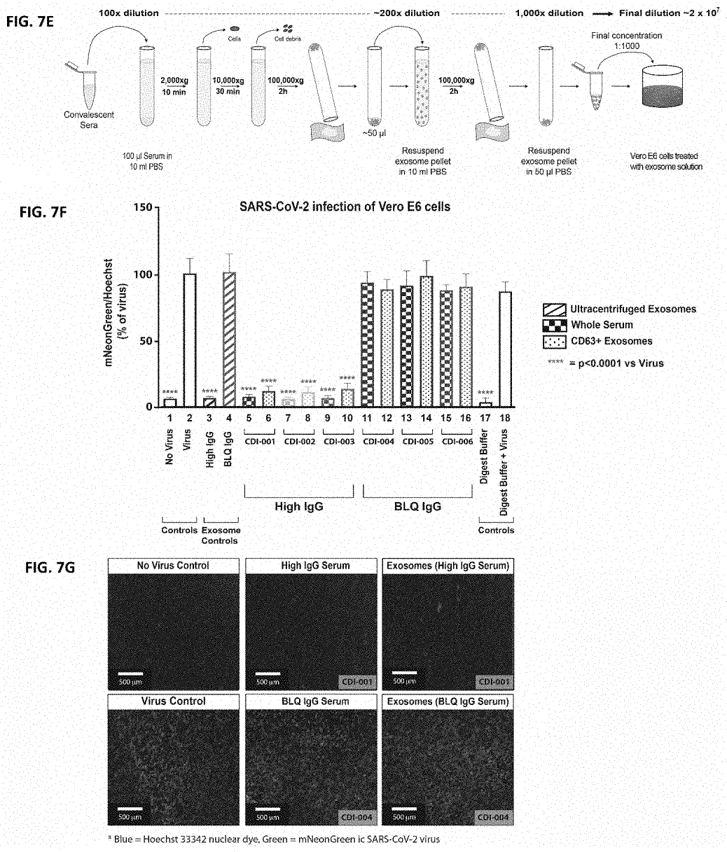

Considering that our previous TEM analyses suggested that a large proportion of exosomes released after EV-CATCHER purification were intact, we sought to determine if exosomes purified from biofluids using EV-CATCHER maintained their biological properties for in vitro analyses. For these proof-of-concept experiments, we established a second pilot study (Convalescent serum sample set), where we purified CD63⁺ circulating exosomes from sera of six individuals who had recently recovered from SARS-CoV-2 infection (enrolled in the convalescent plasma study at HUMC and evaluated for anti-SARS-CoV-2 by ELISA, targeting the Receptor Binding Domain (RBD) of SARS-CoV-2 spike protein as antigen (IgG)). Three of the serum samples were obtained from individuals with high IgG titers (High; as defined by >10,000 dilution of sera) and 3 from serum of individuals with IgG levels below limit of quantification (BLQ; not detectable) as shown in FIG. 7A. We used the EV-CATCHER assay to purify exosomes from the different serum samples and confirmed their identity by Western blot analysis using anti-Alix, -CD9, -CD63 and -CD81 antibodies (FIG. 7B). These experiments revealed homogeneous purification of exosomes between high (FIG. 7B, lanes 1-3) and BLQ sera (FIG. 7B, lanes 4-6). Using TEM we assessed the size, morphology, and integrity of the purified exosomes (FIG. 7C) from both high and BLQ serum samples. Exosome size distribution was also evaluated by nanoparticle tracking (FIG. 7D) and revealed similar nanoparticle distributions (65-150 nm) and quantities between high (average concentration: 1.99×10¹⁰ particles/ml (n=20,481) and BLQ (1.55×10¹⁰ particles/ml (n=20,449) serum exosomes. We performed mNeonGreen SARS-CoV-2 reporter virus (viral replication results in production of measurable green fluorescent protein signal) infection and propagation using gold standard Vero E6, African green monkey kidney, cells, which harbor high levels of the ACE-2 receptor [47], to evaluate the neutralizing properties of the different sera and purified circulating serum exosomes (FIG. 7E) [48-50]. Treatment of VeroE6 cells with whole convalescent sera from individuals with high IgG titers resulted, as anticipated, in neutralization of the SARS-CoV-2 virus (FIG. 7E, High IgG), which was not observed when using whole sera from individuals with BLQ IgG titers (FIG. 7E, BLQ IgG). In order to evaluate potential neutralizing properties of circulating exosomes, we initially obtained highly purified exosomes by the gold-standard ultracentrifugation (UC) approach [35]. In order to prevent serum IgG carry-over, we performed successive ultracentrifugation steps and washes, which we estimated lead to a ˜1-2×10⁷-fold dilution of the initial sera (FIG. 7E; 1 high, 1 BLQ). Our experiments revealed that Vero E6 cells pre-treated with UC exosomes from a high IgG titer serum sample lead to a similar neutralization effect of the SARS-CoV-2 virus (FIG. 7E, third column) as to that seen with whole sera treatment, while cells treated with UC exosomes from the BLQ IgG titer serum sample displayed no neutralizing effect on the virus (FIG. 7E, fourth column) Based on the extremely high level of dilution obtained from UC of sera, our data suggest that neutralizing effects observed with UC exosomes from high IgG titer sera may be exosome-mediated. Therefore, we sought to confirm these unexpected results using serum exosomes purified and released by the EV-CATCHER assay. Our data showed that exosomes purified from high IgG titer sera (FIG. 7E. samples 1-3) exhibited neutralizing activity against SARS-CoV-2, similar to that of UC exosomes from high IgG titer serum, which was not observed with BLQ IgG sera (FIG. 7E. samples 4-6). These observations were validated by the detection of fluorescent viral particles by imaging (FIG. 7F). While these unexpected data are suggestive of neutralizing properties for exosomes purified from convalescent sera with high IgG titers, the mechanism of action remains identified and confirmed, however, our analyses demonstrate that exosomes purified using the EV-CATCHER assay are comparable to those observed with highly-purified UC exosomes from one of the same serum samples.

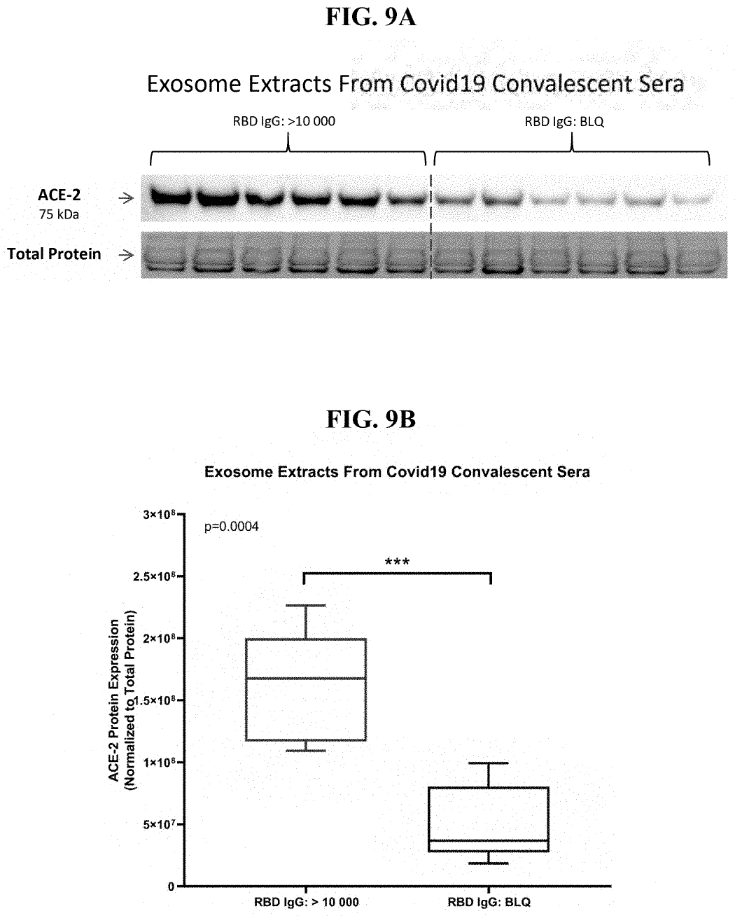

FIG. 9A shows SDS PAGE analysis of exosome extracts from COVID19 convalescent serum (left, high IgG titer convalescent serum, RBD IgG >10,000; right, BLQ convalescent serum) stained to show total protein and ACE-2. FIG. 9B shows a graph of ACE-2 protein expression normalized to total protein for convalescent serum ((left, high IgG titer convalescent serum, RBD IgG >10,000; right, BLQ convalescent serum), depicting the significant increase in ACE-2 in convalescent plasma from individuals with high IgG titers.

### Discussion

In this study we developed EV-CATCHER, a sensitive, customizable antibody-based exosome purification procedure, designed for high-throughput identification of exosomal small-RNA cargos, and the intact release of purified exosomes for evaluation of their biological properties.

A significant issue for identification of circulating miRNA biomarkers is the inherent high level of surrounding miRNA noise, which results in the “dampening” of their signal in biofluids, especially in the detection of miRNA expression changes from a population of cells within a large organism [32]. The evaluation of circulating exosomes provides a unique opportunity to evaluate compartmentalized miRNA cargos from specific cells, however, current laboratory-based technologies have major caveats [25-31]. Thus, we optimized the EV-CATCHER assay with the intent of minimizing RNA background/noise during exosome purification and selected to use a streptavidin coated 96-well plate as binding platform, as we determined that magnetic beads inherently capture RNA and exosomes non-specifically causing interferences with our analyses. Using our optimized small-RNA cDNA library preparation protocol, we demonstrated the specificity of EV-CATCHER by sequencing the RNA extracted from selectively purified CD63+ mouse RAWS264.7 exosomes, which had been spiked into human plasma. Furthermore, next-generation sequencing analyses of different circulating RNA fractions from the same biofluid (whole plasma, ultracentrifuged exosomes from plasma, and antibody-pulled exosomes) further justified the principle that miRNA expression differences can be systematically identified based on the RNA source and the method of purification. These data demonstrate a significant potential for use of the EV-CATCHER assay in the evaluation of human disease pathogenesis by tailoring the antibody selection to the capture of cell-specific surface proteins present on circulating exosomes.

We proposed that a focused selection of encapsulated biomarkers may allow for fine-tuned low-abundance small-RNA detection. Our analyses support this principle as small-RNA sequencing of CD63⁺/CD81⁺/CD9⁺ purified exosomes could identify 10 differentially expressed miRNAs between mildly and severely ill Covid-19 hospitalized patients, whereas none of these miRNAs could be individually detected as differentially expressed when evaluating whole sera (first serum sample set). Interestingly, when we evaluated these 10 miRNAs as an integrative miRNA signature using whole sera miRNA expression sequencing data, we found that we could discriminate the two patient groups (individual pval>0.005). These observations suggest that inherent miRNA expression differences from encapsulated exosomes are dampened when evaluated within a whole-biofluid, further demonstrating the need for focused evaluation of specifically purified exosomes for discovery of low-expressed and potentially robust and clinically relevant miRNA biomarkers. Considering that qPCR remains one of the gold-standards for detection of RNA transcripts in clinical assays, it is important to note that it had significant limitations when working with total RNA extracted from circulating exosomes purified from small serum sample volumes (100 μl). For example, during qPCR validations the top two differentially expressed miRNAs (hsa-miR-146a and hsa-miR126-3p) retained statistical significance between the two patient groups (mild vs severe), whereas hsa-miR-205 lost significance with comparative detection thresholds ˜38-39 amplification cycles. These evaluations would suggest that the detection of low abundance biomarkers will require; 1—the use of more cell-specific antibodies for exosome sub-population enrichment, 2—larger biofluid material inputs, and 3—a pre-amplification step to improve the qPCR detection threshold.

As we developed the EV-CATCHER assay for the discovery of small-RNA biomarkers associated with disease, we ensured the release of intact exosomes for accurate molecular analyses. As such, we chose to include an enzymatically degradable DNA linker between the capture antibody and our low non-specific small-RNA binding platform. For evaluation of the intact release of exosomes, we used TEM and nanoparticle tracking experiments to evaluate enrichment of size-specific intact exosome sub-populations. As our experiments suggest, this enzymatic release of intact exosomes could also afford the opportunity to explore the functional properties of EV-CATCHER purified exosomes using in vitro assays, without precluding in vivo analyses as well. Although we used a pool of well-accepted selection antibodies (anti-CD63/CD81/CD9) for the purification of exosomes as proof of principle for validation of our assay, our data suggest that it has significant potential for selection and analysis of cell-specific exosomes circulating in biofluids by customization with specific monoclonal antibodies. In fact, recent studies have already demonstrated that organ/tissue specific exosomes can be purified from the circulation using monoclonal antibodies, which can then be used for in vitro analyses [51-54]. We propose that to increase specificity of exosome capture, an initial ultrafiltration or ultracentrifugation step may provide intact pooled exosomes, which could then be subsequently subjected to single antibody selection using the EV-CATCHER assay both for miRNA biomarker and functional property evaluations.

As our research institute and its parent institution were located within the early U.S. epicentre of the SARS-CoV-2 pandemic, we sought to determine if our molecular assay could be utilized to evaluate the serum of Covid-19 infected patients. Considering the lack of molecular studies evaluating predictive biomarkers for risk of severe disease stratification for Covid-19 serum specimens, we used the EV-CATCHER assay, as proof-of-principle, to determine if circulating exosomal miRNA expression changes associated with severity of the infection could be detected (first serum sample set). Although on a small-scale, our comparative exosomal miRNA expression analyses between Covid-19 mildly affected (n=13) and severely ill hospitalized patients (n=17) who required mechanical ventilation, allowed for the detection and qPCR validation of at least two downregulated miRNAs (miR-146a and miR-126-3p), which were not detectable in whole sera. These analyses validated the sensitivity of the EV-CATCHER assay, but also suggested that putative prognostic biomarkers associated with severity of the disease may be detectable in serum exosomes of Covid-19 patients. Interestingly, previous studies implicated both of these miRNAs as being immune and vascular regulatory miRNAs, associated with inflammation and injurious vascular events [55-60]. For example, studies by Taganov et al. suggest that hsa-miR-146a is a molecular brake on inflammation [56]. Studies on hsa-miR-126 revealed it to be a vascular miRNA, which regulates angiogenesis [61] in part via activation of VEGF signalling [62]. More precisely, studies demonstrated that exosomes secreted by human endothelial progenitor cells were beneficial to lipopolysaccharide-induced acute lung injury in mice, in part through the delivery of hsa-miRNA-126 (both miR-126-3p and miR-126-5p) into the injured alveolus [63]. Although on a small-scale, our observations that both hsa-miR-146a and hsa-miR-126-3p may be downregulated in severely ill COVID-19 hospitalized patients on mechanical ventilators, when compared to mildly ill hospitalized patients, are consistent with reported anti-inflammatory and vascular health-promoting properties of these regulators. These observations suggest that a larger-scale evaluation of serum exosomes from Covid-19 hospitalized patients may help identify robust prognostic circulating miRNAs, which could be useful to strategically improve clinical outcome by identifying patients who would most benefit from convalescent plasma therapy.

Circulating exosomes have been implicated in the immune response during viral infections [64-68], with studies demonstrating that exosomes can harbor proteins that inhibit viral replication (e.g. against HIV [69]) or proteins which can induce B lymphocyte proliferation (e.g. exosomes released from Epstein-Barr Virus (EBV) infected cells [34]) within their cargo. However, thus far the potential role of circulating exosomes in viral neutralization of SARS-CoV2 has not been evaluated. Our initial experiments showed that highly purified exosomes obtained by successive ultracentrifugation from high IgG titer serum, exhibited neutralizing activity against SARS-CoV-2 infection in vitro, whereas exosomes purified from serum with IgG titer below quantification level did not. We chose to use the EV-CATCHER assay to replicate the neutralizing properties of Covid-19 convalescent exosomes (second serum sample set) in vitro. Using exosomes captured and released by the EV-CATCHER assay from high IgG titer sera, we confirmed this neutralizing activity in vitro, which could not be observed with exosomes from sera with IgG titers below quantification level. Although we may not fully rule out the possibility of minimal IgG carry over (from whole convalescent sera) using our highly specific purification assay, we show that convalescent exosomes highly purified by ultracentrifugation (with estimated dilution factor of ˜1-2×10⁷) exhibit similar viral neutralizing properties than that observed with whole sera. Interestingly, recent studies have demonstrated that exosomes may transport immunoglobulins, as their estimated size of 10-12 nm is compatible with compartmentalization within circulating exosomes [35,36]. Such an exosome property may explain the strong association between exosome neutralization and presence of high neutralizing IgG titers, but will require further evaluation. However, additional mechanisms of viral neutralization have also been proposed, which could include an indirect cloaking strategy wherein convalescent exosomes may bind to cells targeted by the virus and prevent viral entry [65], or direct cloaking strategy where exosomes themselves may harbor viral receptors (i.e. ACE2, the SARS-CoV-2 receptor) to cloak viral particles [70]. Whereas the exact neutralizing mechanism of Covid-19 convalescent exosomes requires further evaluation, our observation that exosomes isolated from high IgG titer sera possess anti-SARS-CoV-2 neutralizing activity has important implications for the potential of purified exosomes as an enhancement to convalescent plasma therapy.

In summary, our study demonstrates that in combination with a sensitive small-RNA cDNA library preparation, a low small-RNA background, reproducible, and customizable exosome selection assay, EV-CATCHER, may help identify low-expressed exosome-specific circulating biomarkers associated with disease. We also propose that this assay may provide a useful tool for evaluation of the biological properties of different exosome sub-populations, both in vitro and possibly in vivo. Considering the 96-plex format of our assay, we propose that it may easily be automated for purification and evaluation of circulating exosomes, with significant prospect of sequential exosome sub-population analysis from a single biofluid sample.

While the present disclosure describes the invention with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

### REFERENCES

1—Mandel P, Metais P. Les acides nucléiques du plasma sanguine chez l′homme. CR Seances Soc. Biol. Fil. 1948; 142:241-243.
2—Schwarzenbach H, Hoon D. S. B., And Pantel K. Cell-free nucleic acids as biomarkers in cancer patients. Nat Rev Cancer. 2011. 11 (6):426-37. doi:10.1038/nrc3066. PMID: 21562580 Review.
3—Cescon, D. W., Bratman, S., Chan, S. M., Siu L. L. Circulating tumor DNA and liquid biopsy in oncology. Nat Cancer 1, 2020; 276-290. doi:10.1038/s43018-020-0043-5.
4—Cabel L, Proudhon C, Romano E, Girard N, Lantz O, Stern M H, Pierga J Y, Bidard F C. Clinical potential of circulating tumour DNA in patients receiving anticancer immunotherapy. Nat Rev Clin Oncol. 2018; 15(10):639-650. doi: 10.1038/s41571-018-0074-3.PMID: 30050094.
5—O'Leary B, Hrebien S, Morden J P, Beaney M, Fribbens C, Huang X, Liu Y, Bartlett C H, Koehler M, Cristofanilli M, Garcia-Murillas I, Bliss J M, Turner N C. Early circulating tumor DNA dynamics and clonal selection with palbociclib and fulvestrant for breast cancer. Nat Commun. 2018; 9(1):896. doi: 10.1038/s41467-018-03215-x.PMID: 2949709.
6—Reece M, Saluja H, Hollington P, Karapetis C S, Vatandoust S, Young G P, Symonds E L. The Use of Circulating Tumor DNA to Monitor and Predict Response to Treatment in Colorectal Cancer. Front Genet. 2019; 10:1118. doi: 10.3389/fgene.2019.01118. eCollection 2019. PMID: 31824558.
7—Larrea E, Sole C, Manterola L, Goicoechea I, Armesto M, Arestin M, Caffarel M M, Araujo A M, Araiz M, Fernandez-Mercado M, Lawrie C H. New Concepts in Cancer Biomarkers: Circulating miRNAs in Liquid Biopsies. Int J Mol Sci. 2016 Apr. 27; 17(5):627. doi: 10.3390/ijms17050627.PMID: 27128908.
8—Cortez M A, Bueso-Ramos C, Ferdin J, Lopez-Berestein G, Sood A K, Calin G A. MicroRNAs in body fluids—the mix of hormones and biomarkers. Nat Rev Clin Oncol. 2011; 8(8):467-77. doi: 10.1038/nrclinonc.2011.76.PMID: 21647195.
9—Zlotorynski E. Insights into the kinetics of microRNA biogenesis and turnover. Nat Rev Mol Cell Biol. 2019; 20(9):511. doi: 10.1038/s41580-019-0164-9.PMID: 31366986.
10—Shah R, Patel T, Freedman J E. Circulating Extracellular Vesicles in Human Disease. N Engl J Med. 2018; 379(10): 958-966. doi:10.1056/NEJMra1704286. PMID: 30184457.
11—LeBleu V S, Kalluri R. Exosomes as a Multicomponent Biomarker Platform in Cancer. Trends Cancer. 2020; S2405-8033(20)30116-3. doi: 10.1016/j.trecan.2020.03.007. PMID: 32307267.
12—Arroyo J. D., Chevillet J. R., Kroh E. M., Ruf I. K., Pritchard C. C., Gibson D. F., Mitchel P. S., Bennett C. F., Pogosova-Agadjanyan E. L., Stirewalt D. L., Tait J F, Tewari M. Argonaute2 complexes carry a population of circulating microRNAs independent of vesicles in human plasma. Proc. Natl. Acad. Sci. USA. 2011; 108:5003-5008. doi: 10.1073/pnas.1019055108.
13—Mitchell P S, Parkin R K, Kroh E M, Fritz B R, Wyman S K, Pogosova-Agadjanyan E L, Peterson A, Noteboom J, O'Briant K C, Allen A, Lin D W, Urban N, Drescher C W, Knudsen B S, Stirewalt D L, Gentleman R, Vessella R L, Nelson P S, Martin D B, Tewari M. Circulating microRNAs as stable blood-based markers for cancer detection. Proc Natl Acad Sci USA. 2008; 105(30):10513-8. doi: 10.1073/pnas.0804549105. PMID: 18663219.
14—Loudig O, Wang T, Ye K, Lin J, Wang Y, Ramnauth A, Liu C, Stark A, Chitale D, Greenlee R, Multerer D, Honda S, Daida Y, Spencer Feigelson H, Glass A, Couch F J, Rohan T, Ben-Dov I Z. Evaluation and Adaptation of a Laboratory-Based cDNA Library Preparation Protocol for Retrospective Sequencing of Archived MicroRNAs from up to 35-Year-Old Clinical FFPE Specimens. Int J Mol Sci. 2017; 18(3):627. doi:10.3390/ijms 18030627. PMID: 28335433.
15—Rohan T E, Wang T, Weinmann S, Wang Y, Lin J, Ginsberg M, Loudig 0. A miRNA Expression Signature in Breast Tumor Tissue Is Associated with Risk of Distant Metastasis. Cancer Res. 2019; 79(7):1705-1713. doi: 10.1158/0008-5472.PMID: 30760517
16—M. Simons, G. Raposo. Exosomes vesicular carriers for intercellular communication. Curr Opin Cell Biol. 2009, 21:575-581. PMID: 1.9447504.
17—Zhang J, Li S, Li L, Li M, Guo C, Yao J, Mi S. Exosome and Exosomal MicroRNA: Trafficking, Sorting, and Function. Genomics Proteomics Bioinformatics. 2015 February; 13(1):17-24. doi: 10.1016/j.gpb.2015.02.001.PMID: 25724326.
18—Fan Q, Yang L, Zhang X, Peng X, Wei S, Su D, Zhai Z, Hua X, Li H. The emerging role of exosome-derived non-coding RNAs in cancer biology. Cancer Lett. 2018; 414:107-115. doi: 10.1016/j.canlet.2017.10.040.PMID: 29107112
19—Wang M, Yu F, Ding H, Wang Y, Li P, Wang K. Emerging Function and Clinical Values of Exosomal MicroRNAs in Cancer. Mol Ther Nucleic Acids. 2019; 16:791-804. doi: 10.1016/j.omtn.2019.04.027. PMID: 31163321.
20—O'Brien K, Breyne K, Ughetto S, Laurent L C, Breakefield X O. RNA delivery by extracellular vesicles in mammalian cells and its applications. Nat Rev Mol Cell Biol. 2020; 26:1-22. doi: 10.1038/s41580-020-0251-y.PMID: 32457507.
21—Colombo M, Raposo G, Théry C. Biogenesis, secretion, and intercellular interactions of exosomes and other extracellular vesicles. Annu Rev Cell Dev Biol. 2014; 30:255-89. doi:10.1146/annurev-cellbio-101512-122326. PMID: 25288114.
22—Andreu Z, Yán̅ez-Mó M. Tetraspanins in extracellular vesicle formation and function. Front Immunol. 2014; 5:442. PMID: 25278937.
23—Mathivanan S, Simpson R J. ExoCarta: A compendium of exosomal proteins and RNA. Proteomics. 2009; 9(21):4997-5000. doi: 10.1002/pmic.200900351. PMID: 19810033
24—Wu D, Yan J, Shen X, Sun Y, Thulin M, Cai Y, Wik L, Shen Q, Oelrich J, Qian X, Dubois K L, Ronquist K G, Nilsson M, Landegren U, Kamali-Moghaddam M. Profiling surface proteins on individual exosomes using a proximity barcoding assay. Nat Commun. 2019; 10(1):3854. doi: 10.1038/s41467-019-11486-1. PMID: 31451692.
25—Larssen P, Wik L, Czarnewski P, Eldh M, Löf L, Ronquist K G, Dubois L, Freyhult E, Gallant C J, Oelrich J, Larsson A, Ronquist G, Villablanca E J, Landegren U, Gabrielsson S, Kamali-Moghaddam M. Tracing Cellular Origin of Human Exosomes Using Multiplex Proximity Extension Assays. Mol Cell Proteomics. 2017; 16(3):502-511. doi: 10.1074/mcp.M116.064725. PMID: 28111361.
26—Yu L L, Zhu J, Liu J X, Jiang F, Ni W K, Qu L S, Ni R Z, Lu C H, Xiao M B. A Comparison of Traditional and Novel Methods for the Separation of Exosomes from Human Samples. Biomed Res Int. 2018; 2018:3634563. doi: 10.1155/2018/3634563. eCollection 2018. PMID: 30148165.
27—Patel G K, Khan M A, Zubair H, Srivastava S K, Khushman M, Singh S, Singh A P. Comparative analysis of exosome isolation methods using culture supernatant for optimum yield, purity and downstream applications. Sci Rep. 2019; 9(1):5335. doi: 10.1038/s41598-019-41800-2. PMID: 30926864.
28—Ludwig A K, De Miroschedji K, Doeppner T R, Borger V, Ruesing J, Rebmann V, Durst S, Jansen S, Bremer M, Behrmann E, Singer B B, Jastrow H, Kuhlmann J D, El Magraoui F, Meyer H E, Hermann D M, Opalka B, Raunser S, Epple M, Horn P A, Giebel B. Precipitation with polyethylene glycol followed by washing and pelleting by ultracentrifugation enriches extracellular vesicles from tissue culture supernatants in small and large scales. J Extracell Vesicles. 2018; 7(1):1528109. doi: 10.1080/20013078.2018.1528109. PMID: 30357008.
29—Macias M, Rebmann V, Mateos B, Varo N, Perez-Gracia J L, Alegre E, Gonzalez A. Comparison of six commercial serum exosome isolation methods suitable for clinical laboratories. Effect in cytokine analysis. Clin Chem Lab Med. 2019; 57(10):1539-1545. doi: 10.1515/cclm-2018-1297. PMID: 30990781.
30—Pugholm L H, Revenfeld A L, Søndergaard E K, Jørgensen MM. Antibody-Based Assays for Phenotyping of Extracellular Vesicles. Biomed Res Int. 2015; 524817. doi: 10.1155/2015/524817. PMID: 26770974.
31—Löf L, Arngården L, Ebai T, Landegren U, Söderberg O, Kamali-Moghaddam M. Detection of Extracellular Vesicles Using Proximity Ligation Assay with Flow Cytometry Readout-ExoPLA. Curr Protoc Cytom. 2017; 81:4.8.1-4.8.10. doi: 10.1002/cpcy.22. PMID: 28678418.
32—Williams Z, Ben-Dov I Z, Elias R, Mihailovic A, Brown M, Rosenwaks Z, Tuschl T. Comprehensive profiling of circulating microRNA via small RNA sequencing of cDNA libraries reveals biomarker potential and limitations. Proc Natl Acad Sci USA. 2013; 110 (10:4255-60. doi: 10.1073/pnas.1214046110. PMID: 23440203.
33—Barros F M, Carneiro F, Machado J C, Melo S A. Exosomes and Immune Response in Cancer: Friends or Foes? Front Immunol. 2018; 9 (730). doi:10.3389/fimmu.2018.00730
34—Gutzeit C, Nagy N, Gentile M, Lyberg K, Gumz J, Vallhov H, Puga I, Klein E, Gabrielsson S, Cerutti A, Scheynius A. Exosomes derived from Burkitt's lymphoma cell lines induce proliferation, differentiation, and class-switch recombination in B cells. J Immunol. 2014; 192(12), 5852-5862. doi: 10.4049/jimmuno1.1302068
35—Burkova E E, Grigor' eva AE, Bulgakov D V, Dmitrenok P S, Vlassov V V, Ryabchikova E I, Sedykh S E, Nevinsky G A. Extra Purified Exosomes from Human Placenta Contain an Unpredictable Small Number of Different Major Proteins. Int J Mol Sci. 2019; 20(10): 2434. doi:10.3390/ijms20102434.
36—Huang C, Fisher K P, Hammer S S, Navitskaya S, Blanchard G J, Busik J V. Plasma Exosomes Contribute to Microvascular Damage in Diabetic Retinopathy by Activating the Classical Complement Pathway. Diabetes. 2018; 67(8): 1639-1649. doi:10.2337/db17-1587.
37—Mallegol J, Van Niel G, Lebreton C, Lepelletier Y, Candalh C, Dugave C, Heath J K, Raposo G, Cerf-Bensussan N, Heyman M. T84-intestinal epithelial exosomes bear MHC class II/peptide complexes potentiating antigen presentation by dendritic cells. Gastroenterology. 2007; 132(5):1866-1876. doi: 10.1053/j.gastro.2007.02.043.+
38—Smith V L, Cheng Y, Bryant B R, Schorey J S. Exosomes function in antigen presentation during an in vivo Mycobacterium tuberculosis infection. Sci Rep. 2017; 7:43578. doi:10.1038/srep43578.
39—Pleat M L, Mathiesen A, DeMarino C, Akpamagbo Y A, Barclay R A, Schwab A, Iordanskiy S, Sampey G C, Lepene B, Nekhai S, Aman M J, Kashanchi F. Ebola VP40 in Exosomes Can Cause Immune Cell Dysfunction [published correction appears in Front Microbiol. 2018 Apr. 17; 9:692]. Front Microbiol. 2016; 7:1765. doi:10.3389/fmicb.2016.01765.
40—Mukhamedova N, Hoang A, Dragoljevic D, Dubrovsky L, Pushkarsky T, Low H, Ditiatkovski M, Fu Y, Ohkawa R, PJ, Horvath. A, Brichachek B, Miller Y1, Murphy A, Bukrinsky N I, Sviridov D. Exosomes containing HIV protein Nef reorganize lipid rafts potentiating inflammatory response in bystander cells. PLoS Pathog. 2019; 15 (7):e1007907. doi: 10.1371/journal.ppat.1007907.
41—Vallhov H, Gutzeit C, Johansson S M, Nagy N, Paul M, Li Q, Friend S, George T C, Klein E, Scheynius A, Gabrielsson S. Exosomes containing glycoprotein 350 released by EMV-transformed B cells selectively target B cells through CD21 and block EBV infection in vitro. J Immunol. 2011; 186 (0:73-82. doi: 10.4049/jimmunol.1001145.
42—Loudig O, Liu C, Rohan T, Ben-Dov I Z. Retrospective MicroRNA Sequencing: Complementary DNA Library Preparation Protocol Using Formalin-fixed Paraffin-embedded RNA Specimens. J Vis Exp. 2018; (135):57471. doi: 10.3791/57471. PMID: 29781987.
43—Mensà E, Giuliani A, Matacchione G, Gur\check{a}u F, Bonfigli A R, Romagnoli F, De Luca M, Sabbatinelli J, Olivieri. Circulating miR-146a in healthy aging and type 2 diabetes: Age- and gender-specific trajectories. Mech Ageing Dev. 2019; 180:1-10. doi: 10.1016/j.mad.2019.03.001.
44—Mazeh H, Deutch T, Karas A, Bogardus K A, Mizrahi I, Gur-Wahnon D, Ben-Dov I Z. Next-Generation Sequencing Identifies a Highly Accurate miRNA Panel That Distinguishes Well-Differentiated Thyroid Cancer from Benign Thyroid Nodules. Cancer Epidemiol Biomarkers Prev. 2018; 27(8):858-863. doi: 10.1158/1055-9965.EPI-18-0055
45—El-Khoury V, Pierson S, Kaoma T, Bernardin F, Berchem G. Assessing cellular and circulating miRNA recovery: the impact of the RNA isolation method and the quantity of input material. Sci Rep 6, 2016; 19529. https://doi.org/10.1038/srep19529
46—Ranieri V M, Rubenfeld G D, Thompson B T, Ferguson N D, Caldwell E, Fan E, Camporota L, Slutsky A S. Acute respiratory distress syndrome: the Berlin Definition. ARDS Definition Task Force, JAMA. 2012; 307(23):2526-33. doi: 10.1001/jama.2012.5669. PMID: 22797452.
47—Hoffmann M, Kleine-Weber H, Schroeder S, Krüger N, Herrler T, Erichsen S, Schiergens T S, Herrler G, Wu N-H, Nitsche A, Müller MA, Drosten C, Pöhlmann S. SARS-CoV-2 Cell Entry Depends on ACE2 and TMPRSS2 and Is Blocked by a Clinically Proven Protease Inhibitor. Cell. 2020; 181(2):271-280. doi: 10.1016/j.cell.2020.02.052.
48—Ng M-L, Tan S-H, See E-E, Ooi E-E, Ling, A-E. Proliferative growth of SARS coronavirus in Vero E6 cells. Journal of General Virology. 2003; 84, 3291-3303. doi; 10.1099/vir.0.19505-0.
49—Yamate M, Yamahita M, Goto T, Tsuji S, Li Y-G, Warachit J, Yunoki M, Ikuta K. Establishment of Vero E6 cell clones persistently infected with severe acute respiratory syndrome coronavirus. Microbes and Infection 7. 2005; 1530-1540. doi: 10.1016/j.micinf.2005.05.013.
50—Xie X, Muruato A, Lokugamage K G, Narayanan K, Zhang X, Zou J, Liu J, Schindewolf C, Bopp N E, Aguilar P V, Plante K S, Weaver S C, Makino S, LeDuc J W, Men. An Infectious cDNA Clone of SARS-CoV-2. Cell Host Microbe. 2020; 27(5):841-848.e3. doi: 10.1016/j.chom.2020.04.004
51—Chen L, Chen R, Kemper S, Brigstock D. R. Pathways of production and delivery of hepatocyte exosomes, J. Cell Commun. Signal, 2018; 12:343-357. doi: 10.1007/s12079-017-0421-7.
52—Vallabhajosyula P, Korutla L, Habertheuer A, Yu M, Rostami S, Yuan C X, Reddy S, Liu C, Korutla V, Koeberlein B, Trofe-Clark J, Rickels M R, Naji A. Tissue-specific exosome biomarkers for noninvasively monitoring immunologic rejection of transplanted tissue. J Clin Invest. 2017 Apr. 3; 127(4):1375-1391. doi: 10.1172/JCI87993. PMID: 28319051.
53—Mathivanan S, Lim J W E, Tauro B J, Ji H, Moritz R L, Simpson R J. Proteomics Analysis of A33 Immunoaffinity-purified Exosomes Released from the Human Colon Tumor Cell Line LIM1215 Reveals a Tissue-specific Protein Signature. Molecular and Cellular Proteomics. 2010; 9(2): 197-208. doi:10.1074/mcp.M900152-MCP200.
54—Lai A, Elfeky O, Rice G E, Salomon C. Optimized Specific Isolation of Placenta-Derived Exosomes from Maternal Circulation. In: Murthi P., Vaillancourt C. (eds) Preeclampsia. Methods in Molecular Biology. 2018; 1710:131-138. doi: 10.1007/978-1-4939-7498-6_10.
55—Taganov K D, Boldin M P, Chang K J, Baltimore D. NF-kappaB-dependent induction of microRNA miR-146, an inhibitor targeted to signaling proteins of innate immune responses. Proc Natl Acad Sci USA. 2006; 103(33):12481-12486. doi:10.1073/pnas.0605298103.
56—Boldin M P, Taganov K D, Rao D S, Yang L, Zhao J L, Kalwani M, Garcia-Flores Y, Luong M, Devrekanli A, Xu J, Sun G, Tay J, Linsley P S, Baltimore D. miR-146a is a significant brake on autoimmunity, myeloproliferation, and cancer in mice. J Exp Med. 2011; 208(6):1189-1201. doi:10.1084/jem.20101823.
57—Gao M, Wang X, Zhang X, Ha T, Ma H, Liu L, Kalbfleisch J H, Gao X, Kao R L, Williams D L, Li C. Attenuation of Cardiac Dysfunction in Polymicrobial Sepsis by MicroRNA-146a Is Mediated via Targeting of IRAK1 and TRAF6 Expression. J Immunol. 2015; 195(2):672-682. doi:10.4049/jimmuno1.1403155.
58—Alexandru N, Andrei E, Safciuc F, Dragan E, Balahura, A M, Badila E, Georgescu A. Intravenous Administration of Allogenic Cell-Derived Microvesicles of Healthy Origins Defend Against Atherosclerotic Cardiovascular Disease Development by a Direct Action on Endothelial Progenitor Cells. Cells. 2020; 9(2):423. doi:10.3390/cells9020423.
59—Arroyo A B, Fernandez-Perez M P, Del Monte A, Águila S, Mendez R, Hernandez-Antolín R, García-Barber N, de Los Reyes-García A, González-Jiménez P, Arcas M I, Vicente V, Menéndez R, Andrés V, González-Conejero R, Martínez C. miR-146a is a pivotal regulator of neutrophil extracellular trap formation promoting thrombosis [published online ahead of print, 2020 Jun. 25]. Haematologica. 2020; haematol.2019.240226. doi:10.3324/haematol.2019.240226.
60—Benbaibeche H, Hichami A, Oudjit B, Haffaf E M, Kacimi G, Koceïr EA, Khan N A. Circulating mir-21 and mir-146a are associated with increased cytokines and CD36 in Algerian obese male participants [published online ahead of print, 2020 Jun. 13]. Arch Physiol Biochem. 2020; 1-6. doi:10.1080/13813455.2020.1775655.
61—Kuhnert F, Mancuso M R, Hampton J, Stankunas K, Asano T, Chen C-Z, Kuo C J. Attribution of vascular phenotypes of the murine Egfl7 locus to the microRNA miR-126. Development. 2008; 135(24):3989-3993. doi:10.1242/dev.029736.
62—Nicoli S, Standley C, Walker P, Hurlstone A, Fogarty K E, Lawson N D. MicroRNA-mediated integration of haemodynamics and Vegf signalling during angiogenesis [published correction appears in Nature. 2010 Sep. 16; 467(7313):356]. Nature. 2010; 464(7292):1196-1200. doi:10.1038/nature08889.
63—Zhou Y, Li P, Goodwin A J, Cook J A, Halushka P V, Chang E, Zingarelli B, Fan H. Exosomes from endothelial progenitor cells improve outcomes of the lipopolysaccharide-induced acute lung injury. Crit Care. 2019; 23(1):44. doi:10.1186/s13054-019-2339-3.
64—Yao Z, Qiao Y, Li X, Chen J, Ding J, Bai J, Shen F, Shi B, Liu J, Li J, Yuan Z. Exosomes Exploit the Virus Entry Machinery and Pathway to Transmit Alpha Interferon-Induced Antiviral Activity. J Virol. 2018; 92 (24):e01578-18. doi:10.1128/JVI.01578-18
65—Gu J, Wu J, Fang D, Qiu Y, Zou X, Jia X, Yin Y, Shen L, Mao L. Exosomes cloak the virion to transmit Enterovirus 71 non-lytically. Virulence. 2020; 11(1):32-38. doi: 10.1080/21505594.2019.1705022.
66—Bedford J G, Infusini G, Dagley L F, Villalon-Letelier F, Zheng M Z M, Bennett-Wood V, Reading P C, Wakim L M. Airway Exosomes Released During Influenza Virus Infection Serve as a Key Component of the Antiviral Innate Immune Response. Front Immunol. 2020; 11:887. doi:10.3389/fimmu.2020.00887
67—Urbanelli L, Buratta T, Tancini B, Sagini K, Delo F, Porcellati S, Emiliani C. The role of extracellular vesicles in viral infection and transmission. Vaccines. 2019; 7 (102). doi:10.3390/vaccines7030102.
68—Jeon H, Yoo S-M, Choi H S, Mun J Y, Kang H-G, Lee J, Park J, Gao S-J, Lee M-S. Extracellular vesicles from KSHV-infected endothelial cells activate the complement system. Oncotarget. 2017; 8(59):99841-99860. doi: 10.18632/oncotarget.21668.
69—Khatua A, Taylor H E, Hildreth J E K, Popik W. Exosomes packaging APOBEC3G confer human immunodeficiency virus resistance to recipient cells. J Virol. 83(2), 512-21 (2009). doi: 10.1128/JVI.01658-08.
70—Wang J, Chen S, Bihl J. Exosome-mediated transfer of ACE-2 (Angiotensin-Converting Enzyme 2) from endothelial progenitor cells promote survival and function of endothelial cell. Oxid Med Cell Longev. 2020; 4213541. doi:10.1155/2020/4213541.

1. A method of preparing a purified population of biological particles from a biological sample from a subject and for evaluating a cargo of the purified population of biological particles comprising:
a) preparing a purified population of biological particles by:
(1) obtaining a biological sample comprising biological particles;
(2) contacting the biological sample comprising biological particles from the subject with a binding agent directed to one or more biological particle surface antigens; wherein the binding agent is linked to a nucleic acid, and wherein the nucleic acid is immobilized on a solid support;
b) isolating the biological particle bound by the binding agent from the biological sample;
c) releasing the biological particle bound to the binding agent;
d) eluting the bound biological particle from the binding agent to form a population of free purified biological particles; and
e) evaluating cargo and surface molecules comprising protein, DNA, RNA or lipids, of the purified population of biological particles,
wherein the isolated biological particles are derived from a healthy subject or a subject suffering from a disease.

2. The method of claim 1, step (e) evaluating cargo and surface molecules further comprising:
(i) identifying proteins and/or lipids specific to a surface of the biological particles by mass spectrometry; or
(ii) identifying protein and/or lipid cargos by mass spectrometry; or
(iii) identifying DNA molecules by sequencing or quantitative PCR; or
(iv) extracting RNA from the purified population of biological particles, and identifying and quantifying expression of small non-coding RNAs comprising microRNAs (miRNAs) encapsulated by the purified population of biological particles.

3. The method of claim 1, comprising an initial ultrafiltration or ultracentrifugation step to provide a starting pooled heterogeneous population of biological particles.

4. The method of claim 1,
(a) wherein the biological sample comprises a body fluid, or
(b) wherein the biological sample comprising a body fluid comprises whole blood, serum, plasma, cerebrospinal fluid (CSF), lymph, urine, feces, sweat, tears, nipple aspirates, or seminal fluid or a secreted biological fluid; or
(c) wherein the body fluid is a circulating or secreted body fluid, or
(d) wherein the body fluid that is a circulating body fluid is whole blood, serum, plasma, cerebrospinal fluid (CSF) or lymph.

5.-7. (canceled)

8. The method of claim 1, wherein the binding agent that binds to one or more biological particle surface antigens is an antibody, an antibody binding fragment, or an aptamer, wherein the aptamer is a nucleic acid or a polypeptide, and wherein the binding agent binds to one or more biological particle surface antigens comprises a dibenzocyclooctyne (DBCO) molecule, 2-IT (2-iminothiolane), MBS (3-maleimidobenzoic acid N-hydroxysuccinimide ester), SPDP (N-succinimidyl 3-(2-pyridyldithio) propionate), SATA (N-succinimidyl S-acetylthioacetate), SMCC (succinimidyl 4-(N-maleimidomethyl) cyclohexane-1-carboxylate), Sulfo-SMCC, or derivatives thereof.

9. (canceled)

10. The method of claim 1,
(a) wherein the biological particle surface antigen comprises one or more of CD9, CD63, CD81, CD37, CD82, Alix, ACE-2, Tim4, PLAP, Adiponectin, FABP4, Caveolin-1, Cytokeratins, EPCAM, E-Cadherin, P63, or heterologous cell surface polypeptides; or
(b) wherein the solid support is a well plate, polymer, or a surface.

11. The method of claim 1,
(a) wherein the nucleic acid comprises DNA, wherein the DNA comprises one or more ribonucleic acid nucleotide, wherein the DNA comprises one or more ribonucleic acid nucleotide, and wherein the DNA comprises a restriction enzyme recognition site; or
(b) wherein the nucleic acid comprises RNA; or
(c) wherein the nucleic acid comprises a DNA/RNA duplex, wherein the nucleic acid is a DNA/RNA duplex which can be degraded by an endonuclease or a RNAse (RNase-H); or
(d) wherein the nucleic acid comprises non-natural nucleotides.

12.-17. (canceled)

18. The method of claim 11, wherein the nucleic acid further comprises a binding moiety on a first end of the nucleic acid and a binding moiety on a second end of the nucleic acid, and wherein the binding moiety on the first end of the nucleic acid and the binding moiety on the second end of the nucleic acid are different.

19. The method of claim 18,
(a) wherein the binding moiety on the first end of the nucleic acid is an avidin, streptavidin or carboxyl binding moiety; or
(b) wherein the binding moiety is biotin; or
(c) wherein the binding moiety on the second end of the nucleic acid is an amine moiety.

20.-24. (canceled)

25. The method of claim 1, wherein releasing the isolated biological particle comprises:
(i) enzymatically cleaving the nucleic acid; or
(ii) displacing a first strand of the nucleic acids connected to the antibody from the second strand of the nucleic acids connected to the support by strand displacement with a complementary nucleic acid to the first or second strand of the nucleic acid and an enzyme having strand displacement activity to release the antibody from the support; or
(iii) separating the annealed DNA strands to allow release of the antibody from the platform without damaging the DNA strand attached to the antibody by a polymerase chain reaction using an oligonucleotide complementary to the region of the DNA attached to the antibody.

26. The method of claim 25,
(a) wherein the enzymatic cleaving is with uracil glycosylase; or
(b) wherein the enzymatic cleaving is with a restriction enzyme; or
(c) wherein the enzymatic cleaving is with an endonuclease or a RNase; or
(d) wherein the enzyme having strand displacement activity is DNA polymerase, topoisomerase, or helicase.

27.-29. (canceled)

30. The method of claim 2, comprising identifying the one or more small non-coding RNAs comprising miRNAs encapsulated in the one or more biological particle by next generation sequencing.

31. (canceled)

32. The method of claim 1, wherein the disease comprises a viral infection, a cancer, abnormal placentation, exercise induced muscle damage, heart failure, Alzheimer's disease, liver cirrhosis, viral and bacterial infection, kidney disease, bone remodeling after injury, wound healing or COPD and asthma.

33. The method of claim 32,
(a) wherein the viral infection is a severe coronavirus infection; or
(b) wherein the severe coronavirus infection is due to SARS-CoV-1, MERS, or SARS-CoV-2; or
(c) wherein the severe coronavirus infection is due to SARS-CoV-2.

34.-35. (canceled)

36. The method of claim 33, wherein the isolated and quantified miRNAs derived from a subject with the severe coronavirus infection due to SARS-CoV-2 include one or more of hsa-miR-146a, hsa-miR-126-3p, hsa-miR-15a, hsa-miR-424, hsa-miR-151-3p, hsa-miR-126-5p, hsa-miR-627-5p, hsa-miR-145, hsa-miR-205, hsa-miR-200c, hsa-miR-550-5p, and hsa-miR-629, and wherein miRNA markers of severe SARSCoV-2 disease include hsa-miR-146a, hsa-miR126-3p or both.

37. (canceled)

38. A method for in vitro evaluation of a subject's risk of developing and for treating a severe coronavirus infection, comprising
a. preparing a purified population of biological particles by:
(1) obtaining a biological sample comprising biological particles from the subject;
(2) contacting the biological sample comprising biological particles from the subject with a binding agent to one or more biological particles surface antigens, wherein the binding agent is linked to a nucleic acid and wherein the nucleic acid is immobilized on a solid support, wherein the binding agent to one or more biological particles surface antigens is an antibody, an antibody fragment or an aptamer;
b. isolating the biological particles bound by the binding agent from the biological sample;
c. releasing the biological particles bound by the binding agent;
d. eluting the bound biological particles from the binding agent to form a population of free purified biological particles;
e. determining a cargo profile for the purified biological particles by evaluating cargo of the purified population of biological particles by:
(i) extracting RNA from the purified population of biological particles;
(ii) identifying and quantifying expression of small non-coding RNAs comprising one or more microRNAs (miRNAs) encapsulated by the purified population of exosomes;
(iii) comparing the cargo profile for the purified biological particle to a cargo profile from a control subject (1) not infected with the coronavirus; (2) infected with the coronavirus who developed mild disease; and (3) infected with the coronavirus who developed severe disease;
(f) determining risk of the patient for the severe viral infection, wherein the small noncoding RNA or protein cargo profile for the purified biological particle is about 1.5-fold lower or about 1.5-fold higher than the cargo profile from the control subject infected with the coronavirus who developed severe disease; and
(g) implementing a therapy appropriate for patients at risk for the severe viral infection.

39. The method of claim 38, comprising an initial ultrafiltration or ultracentrifugation step to provide a pooled heterogeneous population of biological particles.

40. The method of claim 38,
(a) wherein the biological sample comprises a body fluid, or
(b) wherein the biological sample comprises the body fluid comprises whole blood, serum, plasma, cerebrospinal fluid (CSF), lymph, urine, feces, sweat, tears, nipple aspirates, seminal fluid or a secreted biological fluid; or
(c) wherein the body fluid is a circulating or secreted body fluid, wherein the circulating body fluid is whole blood, serum, plasma, CSF, or lymph; or
(d) wherein the severe coronavirus infection is due to SARS-CoV-1, MERS, or SARS-CoV-2; or
(e) wherein the surface antigen comprises one or more of CD9, CD63, CD81, CD37, CD82, Alix, ACE-2, Tim4, PLAP, Adiponectin, FABP4, Caveolin-1, Cytokeratins, EPCAM, E-Cadherin, P63, or heterologous cell surface polypeptides; or
(f) wherein releasing the isolated biological particle comprises:
(i) enzymatically cleaving the nucleic acid, wherein the enzymatic cleaving is with uracil glycosylase, a restriction enzyme, endonuclease or RNase; or
(ii) displacing a first strand of the nucleic acids connected to the antibody from the second strand of the nucleic acids connected to the support by strand displacement with a complementary nucleic acid to the first or second strand of the nucleic acid and an enzyme having strand displacement activity to release the antibody from the support, wherein the enzyme comprising strand displacement activity is DNA polymerase, topoisomerase, or helicase; or
(iii) separating the annealed DNA strands to allow release of the antibody from the platform without damaging the DNA strand attached to the antibody by a polymerase chain reaction using an oligonucleotide complementary to the region of the DNA attached to the antibody; or
(h) wherein identifying and quantifying small noncoding RNAs comprising one or more microRNAs (miRNAs) encapsulated by the purified population of biological particles is by next generation sequencing.

41.-45. (canceled)

46. The method of claim 38,
(a) wherein a population of miRNAs of the subject with the severe coronavirus infection include one or more of hsa-miR-146a, hsa-miR-126-3p, hsa-miR-15a, hsa-miR-424, hsa-miR-151-3p, hsa-miR-126-5p, hsa-miR-627-5p, hsa-miR-145, hsa-miR-205, hsa-miR-200c, hsa-miR-550-5p, or hsa-miR-629 when compared to a control; or
(b) wherein miRNA markers of severe disease caused by SARS-CoV-2 include hsa-miR-146a, hsa-miR126-3p or both hsa-miR-146a and hsa-miR126-3p when compared to a control.

47.-59. (canceled)

60. A method for enhancing therapeutic effectiveness of convalescent plasma therapy for treating a patient at risk for a severe coronavirus infection comprising:
a) preparing a purified population of biological particles from convalescent serum of a convalescent subject by:
(1) obtaining a convalescent serum comprising a high IgG titer against the coronavirus from the convalescent subject;
(2) contacting the convalescent serum with a binding agent directed to one or more biological particles surface antigens; wherein the binding agent is linked to a nucleic acid, and wherein the nucleic acid is immobilized on a solid support, wherein the binding agent to one or more biological particle surface antigens is an antibody, an antibody fragment or an aptamer;
b) isolating the biological particles bound by the binding agent from the biological sample;
c) releasing the biological particles bound to the binding agent;
d) eluting the biological particles from the binding agent to form a population of free purified biological particles;
e) measuring a neutralization titer of the purified biological particles population for the coronavirus in vitro; and
f) administering to the subject the convalescent serum comprising a high titer of neutralizing biological particles and a high titer of neutralizing IgG.

61. The method of claim 60, comprising an initial ultrafiltration or ultracentrifugation step to provide pooled a heterogeneous population of biological particles.

62. The method of claim 60,
(a) wherein the biological sample comprises a body fluid; or
(b) wherein the biological sample comprises a body fluid comprises whole blood, serum, plasma, cerebrospinal fluid (CSF), lymph, urine, feces, sweat, tears, nipple aspirates, seminal fluid or a secreted biological fluid; or
(c) wherein the body fluid is circulating body fluid; or
(d) wherein the body fluid is circulating body fluid and the circulating body fluid comprises whole blood, serum, plasma, cerebrospinal fluid (CSF) or lymph; or
(e) wherein the severe coronavirus infection is due to SARS-CoV-1, MERS, or SARS-CoV-2; or
(f) wherein the biological particle surface antigen comprises one or more of CD9, CD63, CD81, CD37, CD82, Alix, ACE-2, Tim4, PLAP, Adiponectin, FABP4, Caveolin-1, Cytokeratins, EPCAM, E-Cadherin, P63, or heterologous cell surface polypeptides; or
(g) wherein releasing the isolated biological particle comprises:
(i) enzymatically cleaving the nucleic acid, wherein the enzymatic cleaving is with uracil glycosylase, a restriction enzyme, endonuclease, or RNase; or
(ii) displacing a first strand of the nucleic acids connected to the antibody from the second strand of the nucleic acids connected to the support by strand displacement with a complementary nucleic acid to the first or second strand of the nucleic acid and an enzyme having strand displacement activity to release the antibody from the support, wherein the enzyme comprising strand displacement activity is DNA polymerase, topoisomerase, or helicase; or
(iii) separating the annealed DNA strands to allow release of the antibody from the platform without damaging the DNA strand attached to the antibody by a polymerase chain reaction using an oligonucleotide complementary to the region of the DNA attached to the antibody; or
(h) wherein the nucleic acid comprises DNA comprising a ribonucleic acid nucleotide, wherein the ribonucleic acid nucleotide is uracil, or the DNA comprises a restriction enzyme recognition site.

63.-75. (canceled)

76. The method of claim 60, wherein the neutralizing purified biological particle population derived from the convalescent serum with the high IgG titer comprises ACE2-receptors.

77. The method of claim 60, the measuring a neutralization titer of the purified biological particle population for SARS-CoV-2 virus further comprising incubating mammalian cells infected with the coronavirus in vitro with a dilution series of the isolated purified biological particle derived from the convalescent serum with the high IgG titer; and measuring viral particle production compared to a negative control (infected cells without the purified biological particle derived from convalescent serum), wherein viral particle production is decreased by at least 10%, at least 20%, at least 30%, at least 40%, at least 50%, at least 60%, at least 70%, at least 80%, at least 85%, at least 90%, at least 95%, or at least 100% when compared to a negative control.

78. (canceled)

79. The method of claim 60, wherein
(a) the convalescent serum comprising neutralizing purified biological particle may enhance effectiveness of convalescent plasma therapy by at least about 2-fold when compared to treatment with convalescent plasma therapy alone; or
(b) the neutralizing purified biological particle allows presentation of viral antigens to the immune system.

80. A method of treating a subject with a severe coronavirus infection, comprising
(a) determining a neutralization titer of a population of biological particle purified from a biological sample of the subject by:
(1) obtaining the biological sample comprising biological particle;
(2) contacting the biological samples comprising biological particles from the subject with a binding agent to one or more biological particle surface antigens; wherein the binding agent is linked to a nucleic acid, and wherein the nucleic acid is immobilized on a solid support, wherein the binding agent to one or more biological particle surface antigens is an antibody, an antibody fragment or an aptamer;
(3) isolating the biological particles bound by the binding agent from the biological sample;
(4) releasing the biological particles bound to the binding agent;
(5) eluting the bound biological particles from the binding agent to form a population of free purified biological particles;
(6) determining the neutralization titer of the population of purified biological particle in vitro;
(b) selecting the subject to receive convalescent plasma therapy comprising neutralizing purified biological particles when the neutralization titer of the population of purified biological particle purified from the subject is insufficient to decrease viral particle production in vitro;
(c) preparing a purified population of biological particle from convalescent plasma of a convalescent subject by:
(i) obtaining a convalescent serum comprising a high anti-coronavirus IgG titer from a convalescent subject;
(ii) contacting the convalescent serum with a binding agent directed to one or more biological particle surface antigens;
(iii) isolating the biological particle bound by the binding agent from the biological sample;
(iv) releasing the biological particle bound to the binding agent;
(v) eluting the bound biological particle from the binding agent to form a population of free purified biological particle;
(d) measuring a neutralization titer of the biological particle population purified from the convalescent serum for SARS-CoV-2 virus, in vitro; and
(e) administering to the subject the neutralizing purified biological particle alone or the convalescent serum with high IgG titer comprising the neutralizing purified biological particle.

81. The method of claim 80, comprising an initial ultrafiltration or ultracentrifugation step to provide pooled a heterogeneous population of biological particle.

82. The method according to claim 80,
(a) wherein the biological sample comprises a body fluid; or
(b) wherein the biological sample is a body fluid comprising whole blood, serum, plasma, cerebrospinal fluid (CSF), lymph, urine, feces, sweat, tears, nipple aspirates, seminal fluid or a secreted biological fluid; or
(c) wherein the body fluid is circulating body fluid; or
(d) wherein the body fluid is circulating body fluid and the circulating body fluid comprises whole blood, serum, plasma, CSF, or lymph; or
(e) wherein the severe coronavirus infection is due to SARS-CoV-1, MERS, or SARS-CoV-2
(f) wherein the biological particle surface antigen comprises one or more of CD9, CD63, CD81, CD37, CD82, Alix, ACE-2, Tim4, PLAP, Adiponectin, FABP4, Caveolin-1, Cytokeratins, EPCAM, E-Cadherin, P63, or heterologous cell surface polypeptides; or
(g) wherein releasing the isolated biological particle comprises:
(i) enzymatically cleaving the nucleic acid or DNA/RNA hybrids, wherein the enzymatic cleaving is with a restriction enzyme, endonuclease, or RNase; or
(ii) displacing a first strand of the nucleic acids connected to the antibody from the second strand of the nucleic acids connected to the support by strand displacement with a complementary nucleic acid to the first or second strand of the nucleic acid and an enzyme having strand displacement activity to release the antibody from the support, wherein the enzyme comprising strand displacement activity is DNA polymerase, topoisomerase, or helicase; or
(iii) separating the annealed DNA strands to allow release of the antibody from the platform without damaging the DNA strand attached to the antibody by a polymerase chain reaction using an oligonucleotide complementary to the region of the DNA attached to the antibody; or
(h) wherein the nucleic acid comprises DNA comprising a ribonucleic acid nucleotide, the ribonucleic acid nucleotide is uracil, and the enzymatic cleaving is with uracil glycosylase.

83.-94. (canceled)

95. The method of claim 80, the measuring a neutralization titer (i) of the population of biological particles purified from the subject and (ii) of the purified biological particle population from convalescent serum with high IgG titer for the coronavirus further comprising
incubating mammalian cells infected with the coronavirus in vitro with a dilution series of (i) the isolated purified biological particles derived from the subject and (ii) isolated purified biological particles derived from convalescent serum with the high IgG titer; and
comparing viral particle production compared to a negative control (infected cells alone),
wherein a neutralizing population of purified biological particles comprises ACE2-receptors.

96. (canceled)

97. A method of preparing a population of purified cells from a biological sample from a subject comprising:
a) preparing a population of purified cells by:
(1) obtaining a biological sample from the subject comprising cells, wherein the biological sample is prepared in vivo or in vitro;
(2) contacting the biological sample from the subject comprising cells with a binding agent directed to one or more cell surface antigens, wherein the binding agent is linked to a nucleic acid by a linker, and wherein the nucleic acid is immobilized on a solid support, wherein the binding agent to one or more biological particle surface antigens is an antibody, an antibody fragment or an aptamer, wherein the aptamer is a nucleic acid or a polypeptide;
b) isolating the cell bound by the binding agent from the biological sample;
c) releasing the cell bound to the binding agent;
d) eluting the bound cell from the binding agent to form a population of purified cells.

98. The method of claim 97, comprising an initial ultrafiltration or ultracentrifugation step to provide a starting pooled heterogeneous population of cells.

99. (canceled)

100. The method of claim 97,
(a) wherein the biological sample comprises a body fluid; or
(b) wherein the biological sample comprises a body fluid comprising whole blood, serum, plasma, cerebrospinal fluid (CSF), lymph, urine, feces, sweat, tears, nipple aspirates, or seminal fluid or a secreted biological fluid; or
(c) wherein the body fluid is a circulating or secreted body fluid; or
(d) wherein the body fluid is a circulating body fluid and the circulating body fluid comprises whole blood, serum, plasma, cerebrospinal fluid (CSF) or lymph; or
(e) wherein the one or more cell surface antigen comprises CD4, CD8, CD9, CD46, CD63, CD81, CD37, CD82, CD138, CD151, ALix, ACE-2, Tim4, PLAP, Adiponectin, FABP4, Caveolin-1, Cytokeratins, EPCAM, E-Cadherin, P63, or heterologous cell surface polypeptides, wherein the heterologous cell surface polypeptides are expressed by chimeric antigen receptor T cells (CAR-T cells); or
(f) wherein the solid support is a well plate, polymer, or a surface; or
(g) wherein releasing the isolated cell comprises:
(i) enzymatically cleaving the nucleic acid, wherein the enzymatic cleaving is with uracil glycosylase, a restriction enzyme, endonuclease, or RNase; or
(ii) displacing a first strand of the nucleic acids connected to the antibody from the second strand of the nucleic acids connected to the support by strand displacement with a complementary nucleic acid to the first or second strand of the nucleic acid and an enzyme having strand displacement activity to release the antibody from the support, wherein the enzyme having strand displacement activity is DNA polymerase, topoisomerase, or helicase; or
(iii) separating the annealed DNA strands to allow release of the antibody from the platform without damaging the DNA strand attached to the antibody by a polymerase chain reaction using an oligonucleotide complementary to the region of the DNA attached to the antibody.

101.-107. (canceled)

108. The method of claim 97,
(a) wherein the nucleic acid comprises DNA, wherein the DNA comprises one or more ribonucleic acid nucleotide, wherein the one or more ribonucleic acid nucleotide is uracil, and wherein the DNA comprises a restriction enzyme recognition site; or
(b) wherein the nucleic acid comprises RNA; or
(c) wherein the nucleic acid comprises a DNA/RNA duplex, wherein the nucleic acid is a DNA/RNA duplex which can be degraded by an endonuclease or a RNAse (RNase-H); or
(d) wherein the nucleic acid comprises non-natural nucleotides.

109.-114. (canceled)

115. The method of claim 97,
(a) wherein the nucleic acid further comprises a binding moiety on a first end of the nucleic acid and a binding moiety on a second end of the nucleic acid, and wherein the binding moiety on the first end of the nucleic acid and the binding moiety on the second end of the nucleic acid are different,
(i) wherein the binding moiety on the first end of the nucleic acid is an avidin, streptavidin or carboxyl binding moiety; or
(ii) wherein the binding moiety is biotin; or
(iii) wherein the binding moiety on the second end of the nucleic acid is an amine moiety, wherein the amine moiety is azide; or
(b) wherein the binding agent to one or more cell surface antigens comprises a dibenzocyclooctyne (DBCO) molecule, 2-IT (2-iminothiolane), MBS (3-maleimidobenzoic acid N-hydroxysuccinimide ester), SPDP (N-succinimidyl 3-(2-pyridyldithio) propionate), SATA (N-succinimidyl S-acetylthioacetate), SMCC (succinimidyl 4-(N-maleimidomethyl) cyclohexane-1-carboxylate), Sulfo-SMCC, or derivatives thereof.

116.-126. (canceled)

127. The method of claim 97,
(a) wherein the population of purified cells comprises non-eukaryotic cells; or
(b) wherein the population of purified cells comprises eukaryotic cells, wherein the eukaryotic cells are human, mouse, rat, dog, non-human primate, or feline; or
(c) wherein the population of purified cells comprises a combination eukaryotic cells and non-eukaryotic cells; or
(d) wherein the population of purified cells are derived from a healthy subject or a subject suffering from a disease, wherein the disease comprise a cancer, viral infection, abnormal placentation, inflammation and other pathologies, wherein the viral infection is a severe coronavirus infection, wherein the severe coronavirus infection is due to SARS-CoV-1, MERS, or SARS-CoV-2; or
(e) wherein the cells are endothelial cells, epithelial cells, T-cells, fibroblasts, adipocytes, neuronal cells, tumor cells, blood cells, or cardiac cells; or
(f) wherein the population of cells is derived from
(i) non-eukaryotic and eukaryotic species; or
(ii) healthy normal tissue or diseased tissue; or
(iii) a murine orthotopic/xenograft/PDX organ; or
(iv) blood; or
(v) a tissue culture.

128.-135. (canceled)


SEQUENCE LISTING (118 sequences)

SEQ ID NO: 1 - RNA, 47 nt, Artificial Sequence
  Description of Artificial Sequence Synthetic oligonucleotide
aaaaacgauucgagaacgugacugccaugccagcucguacuaucgaa

SEQ ID NO: 2 - RNA, 44 nt, Artificial Sequence
  Description of Artificial Sequence Synthetic oligonucleotide
cgauaguacgagcuggcauggcagucacguucucgaaucguuuu

SEQ ID NO: 3 - RNA, 21 nt, Arabidopsis thaliana
uuuggauugaagggagcucua

SEQ ID NO: 4 - RNA, 22 nt, Homo sapiens
uagcuuaucagacugauguuga

SEQ ID NO: 5 - RNA, 22 nt, Homo sapiens
uaauacugccggguaaugaugg

SEQ ID NO: 6 - RNA, 22 nt, Homo sapiens
ucguaccgugaguaauaaugcg

SEQ ID NO: 7 - RNA, 22 nt, Homo sapiens
ccucugaaauucaguucuucag

SEQ ID NO: 8 - RNA, 21 nt, Homo sapiens
cauuauuacuuuugguacgcg

SEQ ID NO: 9 - RNA, 22 nt, Homo sapiens
uccuucauuccaccggagucug

SEQ ID NO: 10 - RNA, 24 nt, Homo sapiens
ucccugagacccuuuaaccuguga

SEQ ID NO: 11 - RNA, 23 nt, Homo sapiens
uacccuguagaaccgaauuugug

SEQ ID NO: 12 - RNA, 22 nt, Homo sapiens
aaaccguuaccauuacugaguu

SEQ ID NO: 13 - RNA, 21 nt, Homo sapiens
cauuauuacuuuugguacgcg

SEQ ID NO: 14 - RNA, 22 nt, Homo sapiens
uguaaacauccccgacuggaag

SEQ ID NO: 15 - RNA, 22 nt, Homo sapiens
ugagguaguaguuuguacaguu

SEQ ID NO: 16 - RNA, 22 nt, Homo sapiens
uguaaacauccuugacuggaag

SEQ ID NO: 17 - RNA, 22 nt, Homo sapiens
accuggcauacaauguagauuu

SEQ ID NO: 18 - RNA, 22 nt, Homo sapiens
ugagguaguagauuguauaguu

SEQ ID NO: 19 - RNA, 22 nt, Homo sapiens
ugccuacugagcugaaacacag

SEQ ID NO: 20 - RNA, 22 nt, Homo sapiens
uucaaguaauccaggauaggcu

SEQ ID NO: 21 - RNA, 23 nt, Homo sapiens
ugaggggcagagagcgagacuuu

SEQ ID NO: 22 - RNA, 22 nt, Homo sapiens
aguucuucaguggcaagcuuua

SEQ ID NO: 23 - RNA, 22 nt, Homo sapiens
gccuucucuucccgguucuucc

SEQ ID NO: 24 - RNA, 22 nt, Homo sapiens
ugagguaguagguugugugguu

SEQ ID NO: 25 - RNA, 21 nt, Homo sapiens
cauuauuacuuuugguacgcg

SEQ ID NO: 26 - RNA, 22 nt, Homo sapiens
ugagguaguagguuguauaguu

SEQ ID NO: 27 - RNA, 25 nt, Homo sapiens
agugguucuuaacaguucaacaguu

SEQ ID NO: 28 - RNA, 22 nt, Homo sapiens
uccuguacugagcugccccgag

SEQ ID NO: 29 - RNA, 22 nt, Homo sapiens
ggguggggauuuguugcauuac

SEQ ID NO: 30 - RNA, 22 nt, Homo sapiens
acugauuucuuuugguguucag

SEQ ID NO: 31 - RNA, 22 nt, Homo sapiens
agggcuuagcugcuugugagca

SEQ ID NO: 32 - RNA, 22 nt, Homo sapiens
cagugguuuuacccuaugguag

SEQ ID NO: 33 - RNA, 23 nt, Homo sapiens
uacccuguagauccgaauuugug

SEQ ID NO: 34 - RNA, 22 nt, Homo sapiens
gggguuccuggggaugggauuu

SEQ ID NO: 35 - RNA, 22 nt, Homo sapiens
aaaguucugagacacuccgacu

SEQ ID NO: 36 - RNA, 23 nt, Homo sapiens
agcuucuuuacagugcugccuug

SEQ ID NO: 37 - RNA, 23 nt, Homo sapiens
caaagugcuguucgugcagguag

SEQ ID NO: 38 - RNA, 22 nt, Homo sapiens
uagcagcacguaaauauuggcg

SEQ ID NO: 39 - RNA, 22 nt, Homo sapiens
ucccugagacccuaacuuguga

SEQ ID NO: 40 - RNA, 23 nt, Homo sapiens
ugaggggcagagagcgagacuuu

SEQ ID NO: 41 - RNA, 23 nt, Homo sapiens
caacggaaucccaaaagcagcug

SEQ ID NO: 42 - RNA, 22 nt, Homo sapiens
uguaaacauccucgacuggaag

SEQ ID NO: 43 - RNA, 22 nt, Homo sapiens
ugagguaguaguuugugcuguu

SEQ ID NO: 44 - RNA, 22 nt, Homo sapiens
ucgguuaucaugguaccgaugc

SEQ ID NO: 45 - RNA, 21 nt, Homo sapiens
aggcggagacuugggcaauug

SEQ ID NO: 46 - RNA, 22 nt, Homo sapiens
ucaggcucaguccccucccgau

SEQ ID NO: 47 - RNA, 23 nt, Homo sapiens
aacauucaacgcugucggugagu

SEQ ID NO: 48 - RNA, 22 nt, Homo sapiens
cuccugacuccagguccugugu

SEQ ID NO: 49 - RNA, 23 nt, Homo sapiens
uguaaacauccuacacucucagc

SEQ ID NO: 50 - RNA, 22 nt, Homo sapiens
agagcuuagcugauuggugaac

SEQ ID NO: 51 - RNA, 22 nt, Homo sapiens
uggagugugacaaugguguuug

SEQ ID NO: 52 - RNA, 22 nt, Homo sapiens
agagguaguagguugcauaguu

SEQ ID NO: 53 - RNA, 22 nt, Homo sapiens
aguuuugcagguuugcauuuca

SEQ ID NO: 54 - RNA, 21 nt, Homo sapiens
uucaaguaauucaggauaggu

SEQ ID NO: 55 - RNA, 22 nt, Homo sapiens
gggagccaggaaguauugaugu

SEQ ID NO: 56 - RNA, 24 nt, Homo sapiens
uggaagacuagugauuuuguuguu

SEQ ID NO: 57 - RNA, 23 nt, Unknown
  Description of Unknown miR-423-3p sequence
agcucggucugaggccccucagu

SEQ ID NO: 58 - RNA, 22 nt, Unknown
  Description of Unknown miR-186 sequence
caaagaauucuccuuuugggcu

SEQ ID NO: 59 - RNA, 22 nt, Unknown
  Description of Unknown let-7b sequence
ugagguaguagguugugugguu

SEQ ID NO: 60 - RNA, 22 nt, Unknown
  Description of Unknown miR-99a sequence
aacccguagauccgaucuugug

SEQ ID NO: 61 - RNA, 22 nt, Unknown
  Description of Unknown miR-99b sequence
cacccguagaaccgaccuugcg

SEQ ID NO: 62 - RNA, 22 nt, Unknown
  Description of Unknown miR-122 sequence
uggagugugacaaugguguuug

SEQ ID NO: 63 - RNA, 22 nt, Unknown
  Description of Unknown miR-378 sequence
cuccugacuccagguccugugu

SEQ ID NO: 64 - RNA, 22 nt, Unknown
  Description of Unknown miR-93 sequence
cuccugacuccagguccugugu

SEQ ID NO: 65 - RNA, 22 nt, Unknown
  Description of Unknown miR-185 sequence
uggagagaaaggcaguuccuga

SEQ ID NO: 66 - RNA, 21 nt, Unknown
  Description of Unknown miR-184 sequence
uauggaggucucugucugacu

SEQ ID NO: 67 - RNA, 22 nt, Unknown
  Description of Unknown miR-183 sequence
uauggcacugguagaauucacu

SEQ ID NO: 68 - RNA, 22 nt, Unknown
  Description of Unknown miR-34a sequence
uggcagugucuuagcugguugu

SEQ ID NO: 69 - RNA, 22 nt, Unknown
  Description of Unknown miR-132 sequence
aaccguggcuuucgauuguuac

SEQ ID NO: 70 - RNA, 23 nt, Unknown
  Description of Unknown miR-7 sequence
uggaagacuagugauuuuguugu

SEQ ID NO: 71 - RNA, 22 nt, Unknown
  Description of Unknown let-7c sequence
ugagguaguagguuguaugguu

SEQ ID NO: 72 - RNA, 23 nt, Unknown
  Description of Unknown miR-18a sequence
uaaggugcaucuagugcagauag

SEQ ID NO: 73 - RNA, 21 nt, Unknown
  Description of Unknown miR-130b sequence
acucuuucccuguugcacuac

SEQ ID NO: 74 - RNA, 22 nt, Unknown
  Description of Unknown miR-210 sequence
agccacugcccaccgcacacug

SEQ ID NO: 75 - RNA, 23 nt, Unknown
  Description of Unknown miR-146b sequence
ugagaacugaauuccauaggcug

SEQ ID NO: 76 - RNA, 24 nt, Unknown
  Description of Unknown miR-29b sequence
gcugguuucauauggugguuuaga

SEQ ID NO: 77 - RNA, 24 nt, Unknown
  Description of Unknown miR-20a sequence
gcugguuucauauggugguuuaga

SEQ ID NO: 78 - RNA, 23 nt, Unknown
  Description of Unknown miR-194 sequence
uaaagugcuuauagugcagguag

SEQ ID NO: 79 - RNA, 22 nt, Unknown
  Description of Unknown miR-30e* sequence
cuuucagucggauguuuacagc

SEQ ID NO: 80 - RNA, 22 nt, Unknown
  Description of Unknown miR-374b sequence
auauaauacaaccugcuaagug

SEQ ID NO: 81 - RNA, 25 nt, Unknown
  Description of Unknown miR-203 sequence
agugguucuuaacaguucaacaguu

SEQ ID NO: 82 - RNA, 22 nt, Unknown
  Description of Unknown miR-144* sequence
ggauaucaucauauacuguaag

SEQ ID NO: 83 - RNA, 22 nt, Unknown
  Description of Unknown miR-374a sequence
uuauaauacaaccugauaagug

SEQ ID NO: 84 - RNA, 22 nt, Unknown
  Description of Unknown miR-584 sequence
uuaugguuugccugggacugag

SEQ ID NO: 85 - RNA, 22 nt, Unknown
  Description of Unknown miR-143 sequence
ggugcagugcugcaucucuggu

SEQ ID NO: 86 - RNA, 22 nt, Unknown
  Description of Unknown miR-144 sequence
ggauaucaucauauacuguaag

SEQ ID NO: 87 - RNA, 22 nt, Unknown
  Description of Unknown let-7d* sequence
cuauacgaccugcugccuuucu

SEQ ID NO: 88 - RNA, 22 nt, Unknown
  Description of Unknown miR-30b sequence
uguaaacauccuacacucagcu

SEQ ID NO: 89 - RNA, 22 nt, Unknown
  Description of Unknown miR-100 sequence
aacccguagauccgaacuugug

SEQ ID NO: 90 - RNA, 23 nt, Unknown
  Description of Unknown miR-199a-5p sequence
cccaguguucagacuaccuguuc

SEQ ID NO: 91 - RNA, 21 nt, Unknown
  Description of Unknown miR-192 sequence
cugaccuaugaauugacagcc

SEQ ID NO: 92 - RNA, 22 nt, Unknown
  Description of Unknown miR-148a sequence
aaaguucugagacacuccgacu

SEQ ID NO: 93 - RNA, 21 nt, Unknown
  Description of Unknown miR-342 sequence
aggggugcuaucugugauuga

SEQ ID NO: 94 - RNA, 23 nt, Unknown
  Description of Unknown miR-375 sequence
gcgacgagccccucgcacaaacc

SEQ ID NO: 95 - RNA, 22 nt, Unknown
  Description of Unknown miR-409-3p sequence
gaauguugcucggugaaccccu

SEQ ID NO: 96 - RNA, 22 nt, Unknown
  Description of Unknown miR-10b sequence
acccuguagaaccgaauuugug

SEQ ID NO: 97 - RNA, 22 nt, Unknown
  Description of Unknown miR-130a sequence
gcucuuuucacauugugcuacu

SEQ ID NO: 98 - RNA, 21 nt, Unknown
  Description of Unknown miR-126-5p sequence
cauuauuacuuuugguacgcg

SEQ ID NO: 99 - RNA, 22 nt, Unknown
  Description of Unknown miR-150 sequence
ucucccaacccuuguaccagug

SEQ ID NO: 100 - RNA, 24 nt, Unknown
  Description of Unknown miR-155 sequence
uuaaugcuaaucgugauagggguu

SEQ ID NO: 101 - RNA, 22 nt, Unknown
  Description of Unknown miR-32 sequence
uauugcacauuacuaaguugca

SEQ ID NO: 102 - RNA, 23 nt, Unknown
  Description of Unknown miR-34c sequence
aggcaguguaguuagcugauugc

SEQ ID NO: 103 - RNA, 22 nt, Unknown
  Description of Unknown miR-193a-3p sequence
aacuggccuacaaagucccagu

SEQ ID NO: 104 - RNA, 23 nt, Unknown
  Description of Unknown miR-636* sequence
ugugcuugcucgucccgcccgca

SEQ ID NO: 105 - RNA, 22 nt, Unknown
  Description of Unknown miR-1 sequence
acauacuucuuuauaugcccau

SEQ ID NO: 106 - RNA, 21 nt, Unknown
  Description of Unknown miR-133a sequence
gcugguaaaauggaaccaaau

SEQ ID NO: 107 - RNA, 22 nt, Unknown
  Description of Unknown miR-205 sequence
uccuucauuccaccggagucug

SEQ ID NO: 108 - RNA, 22 nt, Unknown
  Description of Unknown miR-200c sequence
cgucuuacccagcaguguuugg

SEQ ID NO: 109 - RNA, 22 nt, Unknown
  Description of Unknown miR-141 sequence
caucuuccagugcaguguugga

SEQ ID NO: 110 - RNA, 21 nt, Unknown
  Description of Unknown miR-200a sequence
caucuuaccggacagugcugg

SEQ ID NO: 111 - RNA, 22 nt, Unknown
  Description of Unknown miR-200b sequence
caucuuacugggcagcauugga

SEQ ID NO: 112 - RNA, 22 nt, Unknown
  Description of Unknown miR-145* sequence
ggauuccuggaaauacuguucu

SEQ ID NO: 113 - RNA, 22 nt, Unknown
  Description of Unknown miR-486 sequence
uccuguacugagcugccccgag

SEQ ID NO: 114 - RNA, 22 nt, Unknown
  Description of Unknown miR-126-3p sequence
ucguaccgugaguaauaaugcg

SEQ ID NO: 115 - RNA, 23 nt, Unknown
  Description of Unknown miR-223 sequence
cguguauuugacaagcugaguug

SEQ ID NO: 116 - RNA, 22 nt, Unknown
  Description of Unknown miR-451 sequence
aaaccguuaccauuacugaguu

SEQ ID NO: 117 - RNA, 22 nt, Unknown
  Description of Unknown miR486 sequence
uccuguacugagcugccccgag

SEQ ID NO: 118 - RNA, 22 nt, Unknown
  Description of Unknown miR-146a sequence
ccucugaaauucaguucuucag